SEQUENCE LISTING (103 sequences)

SEQ ID NO: 1 - DNA, 923 bp, Salmonella enterica
  source 1..923: mol_type = other DNA
atgcatacatccgagttgctaaaacacatttatgacatcaatttgtcatatttactccttgcacagcgtttgatcgtccaggacaaagcatctgcgatgttccgcctcggtatcaacgaagagatggcaaacacactgggcgcgttgaccctgccgcagatggtcaaactggcggagacgaaccagttagtttgtcatttccggtttgacgatcatcagacgatcacccgtttgactcaggattcgcgcgtcgatgacttacagcagattcacacaggtatcatgctttcaacgcgtctgctcaatgaagtggacgatacggcgcgtaagaaaagggcatgataatgagtgaaaaaagcattgttcaggaagctcgcgatatccagttggcgatggagttgattaatcttggcgctcgtctacaaatgctggaaagcgaaacacagctcagccgtggtcgcctcatcaggctgtacaaagaattacgcggtagcccgccgcctaaagggatgctgccattttcgacagactggtttatgacctgggagcaaaatattcatgcctccatgttctgcaacgcctggcaatttttactgaagaccggcttatgcagcggtgtggatgcggtgattaaagcttatcggctttatcttgagcagtgtccgcaaccgcctgaagggccgttgttggcgctgactcgcgcatggacgctggtgcgttttgttgaaagtgggttgcttgaattgtcgagctgtaactgctgcggtgggaactttattacccatgcgcatcagcccgtaggcagctttgcgtgtagtttatgccagccgccatcccgcgcagtaaaaagacgtaaactttcccgagatgctgccgatattattccacaactgctggatgaacagatcgaacaggctgtttaa

SEQ ID NO: 2 - protein, 113 aa, Salmonella enterica
MHTSELLKHIYDINLSYLLLAQRLIVQDKASAMFRLGINEEMANTLGALTLPQMVKLAETNQLVCHFRFDDHQTITRLTQDSRVDDLQQIHTGIMLSTRLLNEVDDTARKKRA

SEQ ID NO: 3 - protein, 192 aa, Salmonella enterica
MSEKSIVQEARDIQLAMELINLGARLQMLESETQLSRGRLIRLYKELRGSPPPKGMLPFSTDWFMTWEQNIHASMFCNAWQFLLKTGLCSGVDAVIKAYRLYLEQCPQPPEGPLLALTRAWTLVRFVESGLLELSSCNCCGGNFITHAHQPVGSFACSLCQPPSRAVKRRKLSRDAADIIPQLLDEQIEQAV

SEQ ID NO: 4 - protein, 295 aa, Salmonella enterica
MLILLGYLVVIGTVFGGYVMTGGHLGALYQPAELVIIGGAGIGAFIVGNNGKAIKGTMKAIPLLFRRSKYTKSMYMDLLALLYRLMAKSRQQGMFSLERDIENPKESEIFASYPRILADAVMLDFIVDYLRLIISGNMNTFEIEALMDEEIETHESEAEVPANSLAMVGDSLPAFGIVAAVMGVVHALASADRPAAELGALIAHAMVGTFLGILLAYGFISPLATVLRQKSAETTKMMQCVKITLLSNLNGYAPPIAVEFGRKTLYSSERPSFIELEEHVRAVRNPNQQQTTEEA

SEQ ID NO: 5 - protein, 309 aa, Salmonella enterica
MKNQAHPIVVVKRRRHKPHGGGAHGSWKIAYADFMTAMMAFFLVMWLISISSPKELIQIAEYFRTPLATAVTGGNRIANSESPIPGGGDDYTQQQGEVEKQPNIDELKKRMEQSRLNKLRGDLDQLIESDPKLRALRPHLKIDLVQEGLRIQIIDSQNRPMFKTGSAEVEPYMRDILRAIAPVLNGIPNRISLAGHTDDFPYANGEKGYSNWELSADRANASRRELVAGGLDNGKVLRVVGMAATMRLSDRGPDDAINRRISLLVLNKQAEQAILHENAESQNEPVSVLQQPAAAPPASVPTSPKAEPR

SEQ ID NO: 6 - protein, 130 aa, Salmonella enterica
MRKWLALLLFPLTVQAAGEGAWQDSGMGVTLNYRGVSASSSPLSARQPVSGVMTLVAWRYELNGPTPAGLRVRLCSQSRCVELDGQSGTTHGFAHVPAVEPLRFVWEVPGGGRLIPALKVRSNQVIVNYR

SEQ ID NO: 7 - protein, 214 aa, Salmonella enterica
MMQPSIKPADEGSAGDIIARIGSLTRMLRDSLRELGLDQAIAEAAEAIPDARDRLDYVVQMTAQAAERALNSVEASQPHQDAMEKEAKALTQRWDEWFDNPIELSDARELVTDTRQFLRDVPGHTSFTNAQLLDIMMAQDFQDLTGQVIKRMMDVIQEIERQLLMVLLENIPEQSARPKRENESLLNGPQVDTSKAGVVASQDQVDDLLDSLGF

SEQ ID NO: 8 - protein, 129 aa, Salmonella enterica
MADKELKFLVVDDFSTMRRIVRNLLKELGFNNVEEAEDGVDALNKLQAGGFGFIISDWNMPNMDGLELLKTIRADSAMSALPVLMVTAEAKKENIIAAAQAGASGYVVKPFTAATLEEKLNKIFEKLGM

SEQ ID NO: 9 - protein, 349 aa, Salmonella enterica
MSKIRVLSVDDSALMRQIMTEIINSHSDMEMVATAPDPLVARDLIKKFNPDVLTLDVEMPRMDGLDFLEKLMRLRPMPVVMVSSLTGKGSEVTLRALELGAIDFVTKPQLGIREGMLAYSEMIAEKVRTAARARIAAHKPMAAPTTLKAGPLLSSEKLIAIGASTGGTEAIRHVLQPLPLSSPAVIITQHMPPGFTRSFAERLNKLCQISVKEAEDGERVLPGHAYIAPGDKHMELARSGANYQIKIHDGPPVNRHRPSVDVLFHSVAKHAGRNAVGVILTGMGNDGAAGMLAMYQAGAWTIAQNEASCVVFGMPREAINMGGVSEVVDLSQVSQQMLAKISAGQAIRI

SEQ ID NO: 10 - protein, 288 aa, Salmonella enterica
MTSSLPSGQTSVLLQMTQRLALSDAHFRRICQLIYQRAGIVLADHKRDMVYNRLVRRLRALGLDDFGRYLSMLEANQNSAEWQAFINALTTNLTAFFREAHHFPILAEHARRRHGEYRVWSAAASTGEEPYSIAITLADALGMAPGRWKVFASDIDTEVLEKARSGIYRLSELKTLSPQQLQRYFMRGTGPHEGLVRVRQELANYVEFSSVNLLEKQYNVPGPFDAIFCRNVMIYFDKTTQEDILRRFVPLLKPDGLLFAGHSENFSNLVREFSLRGQTVYALSKDKA

SEQ ID NO: 11 - protein, 553 aa, Salmonella enterica
MFNRIRVVTMLMMVLGVFALLQLVSGGLLFSSLQHNQQGFVISNELRQQQSELTSTWDLMLQTRINLSRSAARMMMDASNQQSSAKTDLLQNAKTTLAQAAAHYANFKNMTPLPAMAEASANVDEKYQRYQAALAELIQFLDNGNMDAYFAQPTQGMQNALGEALGNYARVSENLYRQTFDQSAHDYRFAQWQLGVLAVVLVLILMVVWFGIRHALLNPLARVITHIREIASGDLTKTLTVSGRNEIGELAGTVEHMQRSLIDTVTQVREGSDAIYSGTSEIAAGNTDLSSRTEQQASALEETAASMEQLTATVKQNADNARQASQLAQSASETARHGGKVVDGVVNTMHEIADSSKKIADIISVIDGIAFQTNILALNAAVEAARAGEQGRGFAVVAGEVRNLASRSAQAAKEIKALIEDSVSRVDTGSVLVESAGETMTDIVNAVTRVTDIMGEIASASDEQSRGIDQVALAVSEMDRVTQQNASLVQESAAAAAALEEQASRLTQAVSAFRLASRPLAVNKPEMRLSVNAQSGNTPQSLAARDDANWETF

SEQ ID NO: 12 - protein, 167 aa, Salmonella enterica
MTGMSNVSKLAGEPSGQEFLVFTLGNEEYGIDILKVQEIRGYDQVTRIANTPAFIKGVTNLRGVIVPIVDLRVKFCEGDVEYDDNTVVIVLNLGQRVVGIVVDGVSDVLSLTAEQIRPAPEFAVTLSTEYLTGLGALGERMLILVNIEKLLNSEEMALLDIAASHVA

SEQ ID NO: 13 - protein, 671 aa, Salmonella enterica
MSMDISDFYQTFFDEADELLADMEQHLLDLVPESPDAEQLNAIFRAAHSIKGGAGTFGFTILQETTHLMENLLDEARRGEMQLNTDIINLFLETKDIMQEQLDAYKNSEEPDAASFEYICNALRQLALEAKGETTPAVVETAALSAAIQEESVAETESPRDESKLRIVLSRLKANEVDLLEEELGNLATLTDVVKGADSLSATLDGSVAEDDIVAVLCFVIEADQIAFEKVVAAPVEKAQEKTEVAPVAPPAVVAPAAKSAAHEHHAGREKPARERESTSIRVAVEKVDQLINLVGELVITQSMLAQRSNELDPVNHGDLITSMGQLQRNARDLQESVMSIRMMPMEYVFSRFPRLVRDLAGKLGKQVELTLVGSSTELDKSLIERIIDPLTHLVRNSLDHGIEMPEKRLEAGKNVVGNLILSAEHQGGNICIEVTDDGAGLNRERILAKAMSQGMAVNENMTDDEVGMLIFAPGFSTAEQVTDVSGRGVGMDVVKRNIQEMGGHVEIQSKQGSGTTIRILLPLTLAILDGMSVRVAGEVFILPLNAVMESLQPREEDLHPLAGGERVLEVRGEYLPLVELWKVFDVDGAKTEATQGIVVILQSAGRRYALLVDQLIGQHQVVVKNLESNYRKVPGISAATILGDGSVALIVDVSALQGLNREQRMAITAA

SEQ ID NO: 14 - protein, 239 aa, Salmonella enterica
MNSLYTAEGVMDKHSLWQRYVPLVRHEALRLQVRLPASVELDDLLQAGGIGLLNAVDRYDALQGTAFTTYAVQRIRGAMLDELRSRDWVPRSVRRNAREVAQAMGQLEQELGRNATETEVAERLGIPVAEYRQMLLDTNNSQLFSYDEWREEHGDSIELVTEEHQQENPLHQLLEGDLRQRVMDAIESLPEREQLVLTLYYQEELNLKEIGAVLEVGESRVSQLHSQAIKRLRTKLGKL

SEQ ID NO: 15 - protein, 266 aa, Salmonella enterica
MKLALLGRQALMGVMAVALVAGMSAKSFADEGLLNKVKERGTLLVGLEGTYPPFSFQGEDGKLTGFEVDFAEALAKHLGVKASLKPTKWDGMLASLDAKRIDVVINQVTISDVRKKKYDFSTPYTVSGIQALVKKGNEGTIKTAADLQGKKVGVGLGTNYEEWLRQHVQGVDIRTYDDDPTKYQDLRVGRIDAILVDRLAALDLVKKTKGTLAVTGDAFSRQESGVALRKGNEDLLKAVDNAIAEMQKDGTLKALSEKWFGADVTQ

SEQ ID NO: 16 - protein, 183 aa, Salmonella enterica
MTVQQPKRRPLSRYLKDFKHSQTHCAHCHKLLDRITLVRRGKIVNKIAISQLDMLLDDAAWQREQKEWVALCRFCGDLHCKKQSDFFDIIGFKQYLFEQTEMSHGTVREYVVRLRRLGNYLSEQNISHDLLQDGFLDESLAPWLPETSTNNYRIALRKYQQYKAHQQIAPRQKSPFTASSDIY

SEQ ID NO: 17 - protein, 506 aa, Salmonella enterica
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 18 - protein, 135 aa, Salmonella enterica
MYTASGIKAYAQVSVESAVMSASPHQLIEMLFDGANSALVRARLFLEQGDVVAKGEALSKAINIIDNGLKAGLDQEKGGEIATNLSELYDYMIRRLLQANLRNDAQAIEEVEGLLSNIAEAWKQISPKASFQESR

SEQ ID NO: 19 - protein, 104 aa, Salmonella enterica
MAAIQGIEGVISQLQATAMAARGQDTHSQSTVSFAGQLHAALDRISDRQAAARVQAEKFTLGEPGIALNDVMADMQKASVSMQMGIQVRNKLVAAYQEVMSMQV

SEQ ID NO: 20 - protein, 560 aa, Salmonella enterica
MSATASTATQPKPLEWLNRLRANPRIPLIVAGSAAVAIVVAMVLWAKTPDYRTLFSNLSDQDGGAIVAQLTQMNIPYRFANGSGAIEVPADKVHELRLRLAQQGLPKGGAVGFELLDQEKFGISQFSEQVNYQRALEGELARTIETLGPVKSARVHLAMPKPSLFVREQKSPSASVTVTLEPGRALDEGQISAVVHLVSSAVAGLPPGNVTLVDQSGHLLTQSNTSGRDLNDAQLKFANDVESRIQRRIEAILSPIVGNGNVHAQVTAQLDFANKEQTEEHYSPNGDASKATLRSRQLNISEQVGAGYPGGVPGALSNQPAPPNEAPIATPPTNQQNAQNTPQTSTSTNSNSAGPRSTQRNETSNYEVDRTIRHTKMNVGDIERLSVAVVVNYKTLADGKPLPLTADQMKQIEDLTREAMGFSDKRGDTLNVVNSPFSAVDNTGGELPFWQQQSFIDQLLAAGRWLLVLVVAWILWRKAVRPQLTRRVEEAKAAQEQAQVRQETEEAVEVRLSKDEQLQQRRANQRLGAEVMSQRIREMSDNDPRVVALVIRQWMSNDHE

SEQ ID NO: 21 - protein, 147 aa, Salmonella enterica
MAQHGALETLKDLAEKEVDDAARLLGEMRRGCQQAEEQLKMLIDYQNEYRSNLNTDMGNGIASNRWINYQQFIQTLEKAIEQHRLQLTQWTQKVDLALKSWREKKQRLQAWQTLQDRQTAAALLAENRMDQKKMDEFAQRAAMRKPE

SEQ ID NO: 22 - protein, 155 aa, Salmonella enterica
MTDSAINKKSKRSIWIPLLVLITLAACATAGYSYWRMQQQPTTNAKAEPAPPPAPVFFALDTFTVNLGDADRVLYIGVTLRLKDEATRARLNEYLPEVRSRLLLLFSRQNAAELSTEAGKQKLIAAIKETLAAPLVAGQPKQVVTDVLYTAFILR

SEQ ID NO: 23 - protein, 334 aa, Salmonella enterica
MGDSILSQAEIDALLNGDSDTKDEPTPGIASDSDIRPYDPNTQRRVVRERLQALEIINERFARQFRMGLFNLLRRSPDITVGAIRIQPYHEFARNLPVPTNLNLIHLKPLRGTGLVVFSPSLVFIAVDNLFGGDGRFPTKVEGREFTHTEQRVINRMLKLALEGYSDAWKAINPLEVEYVRSEMQVKFTNITTSPNDIVVNTPFHVEIGNLTGEFNICLPFSMIEPLRELLVNPPLENSRHEDQNWRDNLVRQVQHSELELVANFADIPLRLSQILKLKPGDVLPIEKPDRIIAHVDGVPVLTSQYGTVNGQYALRVEHLINPILNSLNEEQPK

SEQ ID NO: 24 - protein, 137 aa, Salmonella enterica
MSDMNNPSDENTGALDDLWADALNEQKATTTKSAADAVFQQLGGGDVSGAMQDIDLIMDIPVKLTVELGRTRMTIKELLRLTQGSVVALDGLAGEPLDILINGYLIAQGEVVVVADKYGVRITDIITPSERMRRLSR

SEQ ID NO: 25 - protein, 125 aa, Salmonella enterica
MMKTEATVSQPTAPAGSPLMQVSGALIGIIALILAAAWVIKRMGFAPKGNSVRGLKVSASASLGPRERVVIVEVENARLVLGVTASQINLLHTLPPAENDTEAPVAPPADFQNMMKSLLKRSGRS

SEQ ID NO: 26 - protein, 245 aa, Salmonella enterica
MRRLLFLSLAGLWLFSPAAAAQLPGLISQPLAGGGQSWSLSVQTLVFITSLTFLPAILLMMTSFTRIIIVFGLLRNALGTPSAPPNQVLLGLALFLTFFIMSPVIDKIYVDAYQPFSEQKISMQEALDKGAQPLRAFMLRQTREADLALFARLANSGPLQGPEAVPMRILLPAYVTSELKTAFQIGFTIFIPFLIIDLVIASVLMALGMMMVPPATIALPFKLMLFVLVDGWQLLMGSLAQSFYS

SEQ ID NO: 27 - protein, 89 aa, Salmonella enterica
MTPESVMMMGTEAMKVALALAAPLLLVALITGLIISILQAATQINEMTLSFIPKIVAVFIAIIVAGPWMLNLLLDYVRTLFSNLPYIIG

SEQ ID NO: 28 - protein, 264 aa, Salmonella enterica
MIQVTSEQWLYWLHLYFWPLLRVLALISTAPILSERAIPKRVKLGLGIMITLVIAPSLPANDTPLFSIAALWLAMQQILIGIALGFTMQFAFAAVRTAGEFIGLQMGLSFATFVDPGSHLNMPVLARIMDMLAMLLFLTFNGHLWLISLLVDTFHTLPIGSNPVNSNAFMALARAGGLIFLNGLMLALPVITLLLTLNLALGLLNRMAPQLSIFVIGFPLTLTVGIMLMAALMPLIAPFCEHLFSEIFNLLADIVSEMPINNNP

SEQ ID NO: 29 - protein, 331 aa, Salmonella enterica
MSNLSGTDKSVILLMTIGEDRAAEVFKHLSTREVQALSTAMANVRQISNKQLTDVLSEFEQEAEQFAALNINANEYLRSVLVKALGEERASSLLEDILETRDTTSGIETLNFMEPQSAADLIRDEHPQIIATILVHLKRSQAADILALFDERLRHDVMLRIATFGGVQPAALAELTEVLNGLLDGQNLKRSKMGGVRTAAEIINLMKTQQEEAVITAVREFDGELAQKIIDEMFLFENLVDVDDRSIQRLLQEVDSESLLIALKGAEPPLREKFLRNMSQRAADILRDDLANRGPVRLSQVENEQKAILLIVRRLAETGEMVIGSGEDTYV

SEQ ID NO: 30 - protein, 235 aa, Salmonella enterica
MSNELPWQVWTPDDLAPPPETFVPVEADNVTLTEDTPEPELTAEQQLEQELAQLKIQAHEQGYNAGLAEGRQKGHAQGYQEGLAQGLEQGQAQAQTQQAPIHARMQQLVSEFQNTLDALDSVIASRLMQMALEAARQVIGQTPAVDNSALIKQIQQLLQQEPLFSGKPQLRVHPDDLQRVEEMLGATLSLHGWRLRGDPTLHHGGCKVSADEGDLDASVATRWQELCRLAAPGVL

SEQ ID NO: 31 - protein, 456 aa, Salmonella enterica
MTTRLTRWLTALDNFEAKMALLPAVRRYGRLTRATGLVLEATGLQLPLGATCIIERQDGPETKEVESEVVGFNGQRLFLMPLEEVEGILPGARVYARNGHGDGLQSGKQLPLGPALLGRVLDGGGKPLDGLPAPDTLETGALITPPFNPLQRTPIEHVLDTGVRAINALLTVGRGQRMGLFAGSGVGKSVLLGMMARYTRADVIVVGLIGERGREVKDFIENILGPDGRARSVVIAAPADVSPLLRMQGAAYATRIAEDFRDRGQHVLLIMDSLTRYAMAQREIALAIGEPPATKGYPPSVFAKLPALVERAGNGIHGGGSITAFYTVLTEGDDQQDPIADSARAILDGHIVLSRRLAEAGHYPAIDIEASISRAMTALITEQHYARVRLFKQLLSSFQRNRDLVSVGAYAKGSDPMLDKAITLWPQLEAFLQQGIFERADWEDSLQALDLIFPTV

SEQ ID NO: 32 - protein, 122 aa, Salmonella enterica
MTSTVEFINRWQRIALLSQSLLELAQRGEWDLLLQQEVSYLQSIETVMEKQTPPGITRSIQDMVAGYIKQTLDNEQLLKGLLQQRLDELSSLIGQSTRQKSLNNAYGRLSGMLLVPDAPGAS

SEQ ID NO: 33 - protein, 467 aa, Salmonella enterica
MASISSLGVGSNLPLDQLLTDLTKNEKGRLTPITKQQSANSAKLTAYGTLKSALEKFQTANTALNKADLFKSTVASSTTEDLKVSTTAGAAAGTYKINVTQLAAAQSLATKTTFATTKEQLGDTSVTSRTIKIEQPGRKEPLEIKLDKGDTSMEAIRDAINDADSGIAASIVKVKENEFQLVLTANSGTDNTMKITVEGDTKLNDLLAYDSTTNTGNMQELVKAENAKLNVNGIDIERQSNTVTDAPQGITLTLTKKVTDATVTVTKDDTKAKEAIKSWVDAYNSLVDTFSSLTKYTAVEPGEEASDKNGALLGDSVVRTIQTGIRAQFANSGSNSAFKTMAEIGITQDGTSGKLKIDDDKLTKVLKDNTAAARELLVGDGKETGITTKIATEVKSYLADDGIIDNAQDNVNATLKSLTKQYLSVSNSIDETVARYKAQFTQLDTMMSKLNNTSSYLTQQFTAMNKS

SEQ ID NO: 34 - protein, 495 aa, Salmonella enterica [sequence omitted]

SEQ ID NO: 35 - protein, 506 aa, Salmonella enterica; identical to SEQ ID NO: 17

SEQ ID NO: 36 - protein, 140 aa, Salmonella enterica
MTRLSEILDQMTTVLNDLKTVMDAEQQQLSVGQINGSQLQRITEEKSSLLATLDYLEQQRRLEQNAQRSANDDIAERWQAITEKTQHLRDLNQHNGWLLEGQIERNQQALEVLKPHQEPTLYGADGQTSVSHRGGKKISI

SEQ ID NO: 37 - protein, 97 aa, Salmonella enterica
MSIDRTSPLKPVSTVQTRETSDTPVQKTRQEKTSAATSASVTLSDAQAKLMQPGVSDINMERVEALKTAIRNGELKMDTGKIADSLIREAQSYLQSK

SEQ ID NO: 38 - protein, 219 aa, Salmonella enterica
MQTLKRGFAVAALLFSPLTMAQDINAQLTTWFSQRLAGFSDEVVVTLRSSPNLLPSCEQPAFSMTGSAKLWGNVNVVARCANEKRYLQVNVQATGNYVAVAAPIARGGKLTPANVTLKRGRLDQLPPRTVLDIRQIQDAVSLRDLAPGQPVQLTMIRQAWRVKAGQRVQVIANGEGFSVNAEGQAMNNAAVAQNARVRMTSGQIVSGTVDSDGNILINL

SEQ ID NO: 39 - protein, 138 aa, Salmonella enterica
MLDRLDAALRFQQEALNLRAQRQEILAANIANADTPGYQARDIDFASELKKVMVRGREETGGVALTLTSSHHIPAQAVSSPAVDLLYRVPDQPSLDGNTVDMDRERTQFADNSLKYQMGLTVLGSQLKGMMNVLQGGN

SEQ ID NO: 40 - protein, 134 aa, Salmonella enterica
MALLNIFDIAGSALAAQSKRLNVAASNLANADSVTGPDGQPYRAKQVVFQVDAAPGQATGGVKVASVIESQAPEKLVYEPGNPLADANGYVKMPNVDVVGEMVNTMSASRSYQANIEVLNTVKSMMLKTLTLGQ

SEQ ID NO: 41 - protein, 232 aa, Salmonella enterica
MSIAVNMNDPTNTGVKTTTGSGSMTGSNAADLQSSFLTLLVAQLKNQDPTNPLQNNELTTQLAQISTVSGIEKLNTTLGAISGQIDNSQSLQATTLIGHGVMVPGTTILAGKGAEEGAVTSTTPFGVELQQPADKVTATITDKDGRVVRTLEIGELRAGVHTFTWDGKQTDGTTVPNGSYNIAITASNGGTQLVAQPLQFALVQGVTKGSNGNLLDLGTYGTTTLDEVRQII

SEQ ID NO: 42 - protein, 403 aa, Salmonella enterica
MSFSQAVSGLNAAATNLDVIGNNIANSATYGFKSGTASFADMFAGSKVGLGVKVAGITQDFTDGTTTNTGRGLDVAISQNGFFRLVDSNGSVFYSRNGQFKLDENRNLVNMQGMQLTGYPATGTPPTIQQGANPAPITIPNTLMAAKSTTTASMQINLNSTDPVPSKTPFSVSDADSYNKKGTVTVYDSQGNAHDMNVYFVKTKDNEWAVYTHDSSDPAATAPTTASTTLKFNENGILESGGTVNITTGTINGATAATFSLSFLNSMQQNTGANNIVATNQNGYKPGDLVSYQINNDGTVVGNYSNEQEQVLGQIVLANFANNEGLASQGDNVWAATQASGVALLGTAGSGNFGKLTNGALEASNVDLSKELVNMIVAQRNYQSNAQTIKTQDQILNTLVNLR

SEQ ID NO: 43 - protein, 251 aa, Salmonella enterica
MDHAIYTAMGAASQTLNQQAVTASNLANASTPGFRAQLNALRAVPVDGLSLATRTLVTASTPGADMTPGQLDYTSRPLDVALQQDGWLVVQAADGAEGYTRNGNIQVGPTGQLTIQGHPVIGEGGPITVPEGSEITIAADGTISALNPGDPPNTVAPVGRLKLVKAEGNEVQRSDDGLFRLTAEAQAERGAVLAADPSIRIMSGVLEGSNVKPVEAMTDMIANARRFEMQMKVITSVDENEGRANQLLSMS

SEQ ID NO: 44 - protein, 260 aa, Salmonella enterica
MISSLWIAKTGLDAQQTNMDVIANNLANVSTNGFKRQRAVFEDLLYQTIRQPGAQSSEQTTLPSGLQIGTGVRPVATERLHSQGNLSQTNNSKDVAIKGQGFFQVMLPDGTSAYTRDGSFQVDQNGQLVTAGGFQVQPAITIPANALSITIGRDGVVSVTQQGQAAPVQVGQLNLTTFMNDTGLESIGENLYIETQSSGAPNESTPGLNGAGLLYQGYVETSNVNVAEELVNMIQVQRAYEINSKAVSTTDQMLQKLTQL

SEQ ID NO: 45 - protein, 232 aa, Salmonella enterica
MQKYALHAYPVMALMVATLTGCAWIPAKPLVQGATTAQPIPGPVPVANGSIFQSAQPINYGYQPLFEDRRPRNIGDTLTIVLQENVSASKSSSANASRDGKTSFGFDTVPRYLQGLFGNSRADMEASGGNSFNGKGGANASNTFSGTLTVTVDQVLANGNLHVVGEKQIAINQGTEFIRFSGVVNPRTISGSNSVPSTQVADARIEYVGNGYINEAQNMGWLQRFFLNLSPM

SEQ ID NO: 46 - protein, 365 aa, Salmonella enterica
MFKALAGIVLALVATLAHAERIRDLTSVQGVRENSLIGYGLVVGLDGTGDQTTQTPFTTQTLNNMLSQLGITVPTGTNMQLKNVAAVMVTASYPPFARQGQTIDVVVSSMGNAKSLRGGTLLMTPLKGVDSQVYALAQGNILVGGAGASAGGSSVQVNQLNGGRITNGAIIERELPTQFGAGNTINLQLNDEDFTMAQQITDAINRARGYGSATALDARTVQVRVPSGNSSQVRFLADIQNMEVNVTPQDAKVVINSRTGSVVMNREVTLDSCAVAQGNLSVTVNRQLNVNQPNTPFGGGQTVVTPQTQIDLRQSGGSLQSVRSSANLNSVVRALNALGATPMDLMSILQSMQSAGCLRAKLEII

SEQ ID NO: 47 - protein, 316 aa, Salmonella enterica
MIGDGKLLASAAWDAQSLNELKAKAGQDPAANIRPVARQVEGMFVQMMLKSMREALPKDGLFSSDQTRLYTSMYDQQIAQQMTAGKGLGLADMMVKQMTSGQTMPADDAPQVPLKFSLETVNSYQNQALTQLVRKAIPKTPDSSDAPLSGDSKDFLARLSLPARLASEQSGVPHHLILAQAALESGWGQRQILRENGEPSYNVFGVKATASWKGPVTEITTTEYENGEAKKVKAKFRVYSSYLEALSDYVALLTRNPRYAAVTTAATAEQGAVALQNAGYATDPNYARKLTSMIQQLKAMSEKVSKTYSANLDNLF

SEQ ID NO: 48 - protein, 553 aa, Salmonella enterica
MSSLINHAMSGLNAAQAALNTVSNNINNYNVAGYTRQTTILAQANSTLGAGGWIGNGVYVSGVQREYDAFITNQLRGAQNQSSGLTTRYEQMSKIDNLLADKSSSLSGSLQSFFTSLQTLVSNAEDPAARQALIGKAEGLVNQFKTTDQYLRDQDKQVNIAIGSSVAQINNYAKQIANLNDQISRMTGVGAGASPNDLLDQRDQLVSELNKIVGVEVSVQDGGTYNLTMANGYTLVQGSTARQLAAVPSSADPTRTTVAYVDEAAGNIEIPEKLLNTGSLGGLLTFRSQDLDQTRNTLGQLALAFADAFNAQHTKGYDADGNKGKDFFSIGSPVVYSNSNNADKTVSLTAKVVDSTKVQATDYKIVFDGTDWQVTRTADNTTFTATKDADGKLEIDGLKVTVGTGAQKNDSFLLKPVSNAIVDMNVKVTNEAEIAMASESKLDPDVDTGDSDNRNGQALLDLQNSNVVGGNKTFNDAYATLVSDVGNKTSTLKTSSTTQANVVKQLYKQQQSVSGVNLDEEYGNLQRYQQYYLANAQVLQTANALFDALLNIR

SEQ ID NO: 49 - protein, 317 aa, Salmonella enterica
MRISTQMMYEQNMSGITNSQAEWMKLGEQMSTGKRVTNPSDDPIAASQAVVLSQAQAQNSQYALARTFATQKVSLEESVLSQVTTAIQTAQEKIVYAGNGTLSDDDRASLATDLQGIRDQLMNLANSTDGNGRYIFAGYKTEAAPFDQATGGYHGGEKSVTQQVDSARTMVIGHTGAQIFNSITSNAVPEPDGSDSEKNLFVMLDTAIAALKTPVEGNNVEKEKAAAAIDKTNRGLKNSLNNVLTVRAELGTQLSELSTLDSLGSDRALGQKLQMSNLVDVDWNSVISSYVMQQAALQASYKTFTDMQGMSLFQLNR

SEQ ID NO: 50 - protein, 133 aa, unidentified
MSEEGFMLAVLKGIPLIQDIRAEGNSRSWIMTIDGHPARGEIFSEAFSISLFLNDLESLPKPCLAYVTLLLAAHPDVHDYAIQLTADGGWLNGYYTTSSSSELIAIEIEKHLALTCILKNVIRNHHKLYSGGV

SEQ ID NO: 51 - protein, 260 aa, Una virus
MKIHIPSAASNIVDGNSPPSDIQAKEVSFPPPEIPAPGTPAAPVLLTPEQIRQQRDYAIHFMQYTIRALGATVVFGLSVAAAVISGGAGLPIAILAGAALVIAIGDACCAYHNYQSICQQKEPLQTASDSVALVVSALALKCGASLNCANTLANCLSLLIRSGIAISMLVLPLQFPLPAAENIAASLDMGSVITSVSLTAIGAVLDYCLARPSGDDQENSVDELHADPSVLLAEQMAALCQSATTPALMDSSDHTSRGEP

SEQ ID NO: 52 - protein, 229 aa, unidentified
MKPVSPNAQVGGQRPVNAPEESPPCPSLPHPETNMESGRIGPQQGKERVLAGLAKRVIECFPKEIFSWQTVILGGQILCCSAGIALTVLSGGGAPLVALAGIGLAIAIADVACLIYHHKHHLPMAHDSIGNAVFYIANCFANQRKSMAIAKAVSLGGRLALTATVMTHSYWSGSLGLQPHLLERLNDITYGLMSFTRFGMDGMAMTGMQVSSPLYRLLAQVTPEQRAPE

SEQ ID NO: 53 - protein, 322 aa, unidentified
MPFHIGSGCLPAIISNRRIYRIAWSDTPPEMSSWEKMKEFFCSTHQAEALECIWTICHPPAGTTREDVVSRFELLRTLAYDGWEENIHSGLHGENYFCILDEDSQEILSVTLDDVGNYTVNCQGYSETHHLTMATEPGVERTDITYNLTSDIDAAAYLEELKQNPIINNKIMNPVGQCESLMTPVSNFMNEKGFDNIRYRGIFIWDKPTEEIPTNHFAVVGNKEGKDYVFDVSAHQFENRGMSNLNGPLILSADEWVCKYRMATRRKLIYYTDFSNSSIAANAYDALPRELESESMAGKVFVTSPRWFNTFKKQKYSLIGKM

SEQ ID NO: 54 - protein, 408 aa, unidentified
MPLSVGQGYFTSSISSEKFNAIKESARLPELSLWEKIKAYFFTTHHAEALECIFNLYHHQELNLTPVQVRGAYIKLRALASQGCKEQFIIESQEHADKLIIKDDNGENILSIEVECHPEAFGLAKEINKSHPKPKNISLGDITRLVFFGDSLSDSLGRMFEKTHHILPSYGQYFGGRFTNGFTWTEFLSSPHFLGKEMLNFAEGGSTSASYSCFNCIGDFVSNTDRQVASYTPSHQDLAIFLLGANDYMTLHKDNVIMVVEQQIDDIEKIISGGVNNVLVMGIPDLSLTPYGKHSDEKRKLKDESIAHNALLKTNVEELKEKYPQHKICYYETADAFKVIMEAASNIGYDTENPYTHHGYVHVPGAKDPQLDICPQYVFNDLVHPTQEVHHCFAIMLESFIAHHYSTE

SEQ ID NO: 55 - DNA, 1227 bp, unidentified
  source 1..1227: mol_type = other DNA
atgccattgagtgttggacagggttatttcacatcatctatcagttctgaaaaatttaatgcgataaaagaaagcgcacgccttccggaattaagtttatgggagaaaatcaaagcatatttctttaccacccaccatgcagaggcgctcgaatgtatctttaatctttaccaccatcaggaactgaatctaacaccggtacaggttcgcggagcctacatcaaacttcgagccttagcgtctcagggatgtaaagaacagtttattatagaatcacaggaacacgccgataagttgattattaaagatgataatggtgaaaatattttgtctattgaggttgaatgtcatccggaagcttttggtcttgcaaaagaaatcaataaatcacatcccaagcccaaaaatatttctttgggtgatattaccagactggtattttttggcgacagcttgtctgactccttagggcgtatgtttgaaaaaacacatcatatcttaccctcctatggtcaatactttggcggaaggtttactaatggatttacctggactgagtttttatcatctccacacttcttaggtaaagagatgcttaattttgctgaagggggaagtacatcggcaagctattcctgctttaattgcatcggtgactttgtatcaaatacggacagacaagtcgcatcttacaccccttctcaccaggacctggcgatatttttattgggggctaatgactatatgacactacacaaagataatgtaataatggtcgttgagcaacaaattgatgatattgaaaaaataatttccggtggagttaataatgttctggtcatggggattcccgatttgtctttaacaccttatggcaaacattctgatgaaaaaagaaagcttaaggatgaaagcatcgctcacaatgccctgttaaaaactaatgttgaagaattaaaagaaaaatacccccagcataaaatatgctattacgagactgccgatgcatttaaggtgataatggaggcggccagtaatattggttatgatacggaaaacccttatactcaccacggctatgtacatgttcccggggctaaagaccctcagctagatatatgtccgcaatacgtcttcaacgaccttgtccatccaacccaggaagtccatcattgttttgccataatgttagaaagttttatagctcatcattattccactgaataa

SEQ ID NO: 56 - protein, 408 aa, unidentified; identical to SEQ ID NO: 54

SEQ ID NO: 57 - protein, 336 aa, unidentified
MIPPLNRYVPALSKNELVKTVTNRDIQFTSFNGKDYPLCFLDEKTPLLFQWFERNPARFGKNDIPIINTEKNPYLNNIIKAATIEKERLIGIFVDGDFFPGQKDAFSKLEYDYENIKVIYRNDIDFSMYDKKLSEIYMENISKQESMPEEKRDCHLLQLLKKELSDIQEGNDSLIKSYLLDKGHGWFDFYRNMAMLKAGQLFLEADKVGCYDLSTNSGCIYLDADMIITEKLGGIYIPDGIAVHVERIDGRASMENGIIAVDRNNHPALLAGLEIMHTKFDADPYSDGVCNGIRKHFNYSLNEDYNSFCDFIEFKHDNIIMNTSQFTQSSWARHVQ

SEQ ID NO: 58 - protein, 348 aa, unidentified
MARFNAAFTRIKIMFSRIRGLISCQSNTQTIAPTLSPPSSGHVSFAGIDYPLLPLNHQTPLVFQWFERNPDRFGQNEIPIINTQKNPYLNNIINAAIIEKERIIGIFVDGDFSKGQRKALGKLEQNYRNIKVIYNSDLNYSMYDKKLTTIYLENITKLEAQSASERDEVLLNGVKKSLEDVLKNNPEETLISSHNKDKGHLWFDFYRNLFLLKGSDAFLEAGKPGCHHLQPGGGCIYLDADMLLTDKLGTLYLPDGIAIHVSRKDNHVSLENGIIAVNRSEHPALIKGLEIMHSKPYGDPYNDWLSKGLRHYFDGSHIQDYDAFCDFIEFKHENIIMNTSSLTASSWR

SEQ ID NO: 59 - protein, 336 aa, unidentified
MPITIGNGFLKSEILTNSPRNTKEAWWKVLWEKIKDFFFSTGKAKADRCLHEMLFAERAPTRERLTEIFFELKELACASQRDRFQVHNPHENDATIILRIMDQNEENELLRITQNTDTFSCEVMGNLYFLMKDRPDILKSHPQMTAMIKRRYSEIVDYPLPSTLCLNPAGAPILSVPLDNIEGYLYTELRKGHLDGWKAQEKATYLAAKIQSGIEKTTRILHHANISESTQQNAFLETMAMCGLKQLEIPPPHTHIPIEKMVKEVLLADKTFQAFLVTDPSTSQSMLAEIVEAISDQVFHAIFRIDPQAIQKMAEEQLTTLHVRSEQQSGCLCCFL

SEQ ID NO: 60 - protein, 316 aa, Una virus
MPITIGRGFLKSEMFSQSAISQRSFFTLLWEKIKDFFCDTQRSTADQYIKELCDVASPPDAQRLFDLFCKLYELSSPSCRGNFHFQHYKDAECQYTNLCIKDGEDIPLCIMIRQDHYYYEIMNRTVLCVDTQSAHLKRYSDINIKASTYVCEPLCCLFPERLQLSLSGGITFSVDLKNIEETLIAMAEKGNLCDWKEQERKAAISSRINLGIAQAGVTAIDDAIKNKIAAKVIENTNLKNAAFEPNYAQSSVTQIVYSCLFKNEILMNMLEESSSHGLLCLNELTEYVTLQVHNSLFSEDLSSLVETTKNEAHHQS

SEQ ID NO: 61 - protein, 291 aa, unidentified
MPITNASPENILRYLHAAGTGTKEAMKSATSPRGILEWFVNFFTCGGVRRSNERWFREVIGKLTTSLLYVNKNAFFDGNKIFLEDVNGCTICLSCGAASENTDPMVIIEVNKNGKTVTDKVDSERFWNVCRMLKLMSKHNIQQPDSLITEDGFLNLRGVNLAHKDFQGEDLSKIDASNADFRETTLSNVNLVGANLCCANLHAVNLMGSNMTKANLTHADLTCANMSGVNLTAAILFGSDLTDTKLNGAKLDKIALTLAKALTGADLTGSQHTPTPLPDYNDRTLFPHPIF

SEQ ID NO: 62 - protein, 350 aa, unidentified
MERSLDSLAGMAKSAFGAGTSAAMRQATSPKTILEYIINFFTCGGIRRRNETQYQELIETMAETLKSTMPDRGAPLPENIILDDMDGCRVEFNLPGENNEAGQVIVRVSKGDHSETREIPLASFEKICRALLFRCEFSLPQDSVILTAQGGMNLKGAVLTGANLTSENLCDADLSGANLEGAVLFMADCEGANFKGANLSGTSLGDSNFKNACLEDSIMCGATLDHANLTGANLQHASLLGCSMIECNCSGANMDHTNLSGATLIRADMSGATLQGATIMAAIMEGAVLTRANLRKASFISTNLDGADLAEANLNNTCFKDCTLTDLRTEDATMSTSTQTLFNEFYSENI

SEQ ID NO: 63 - protein, 319 aa, unidentified
MPVTLSFGNRHNYEINHSRLARLMSPDKEEALYMGVWDRFKDCFRTHKKQEVLEVLYTLIHGCERENQAELNVDITGMEKIHAFTQLKEYANPSQQDRFVMRFDMNQTQVLFEIDGKVIDKCNLHRLLNVSENCIFKVMEEDEEELFLKICIKYGEKISRYPELLEGFANKLKDAVNEDDDVKDEVYKLMRSGEDRKMECVEWNGTLTEEEKNKLRCLQMGSFNITTQFFKIGYWELEGEVLFDMVHPTLSYLLQAYKPSLSSDLIETNTMLFSDVLNKDYDDYQNNKREIDAILRRIYRSHNNTLFISEKSSCRNMLI

SEQ ID NO: 64 - protein, 480 aa, unidentified
MQYAYTSNEATSNLELLNKWRIESPDIEKEERNSIYDKIIEANHTGSLSITAHHVTSIPVFPDNLSELNLSSCYTLESIPNLPDGLKSLTISGNQTIKISYFPDSLESLSIDMQAYEENYTFPALPYGLKSFTACYGKFLPPLPPHLSSLSLQNFSEILCAELPYKLDKLDLQNCPFLPLMKMLPEELKELSIELIRTVPGTVIDDILPDKLKKLSINFCDNIKLPVKLPVNLKSINLSSRTPIAWEIPTCNLPAHIDISTDGYVKLNPEFLTRSDITFSNKPAGDVLSFQPGDVVYGLCKARDRVNTLVNSLYYFSKKDIIIQNTLTDAVWDRKNRAVFNKDEKIAERLNDVQRGIFFREFLSQHKKYNITEDKYSDLSNEECWIKTSKAGLEFQTRLRERSVIFVIDNLVDAISDIANKTGKHGNSITAHELRWVYRNRHDDLVKQNVKFFLNGEAISHEDVFSLVGWDKYKPKNRNR

SEQ ID NO: 65 - protein, 317 aa, unidentified
MSDEALTLLFSAVENGDQNCIDLLCNLALRNDDLGHRVEKFLFDLFSGKRTGSSDIDKKINQACLVLHQIANNDITKDNTEWKKLHAPSRLLYMAGSATTDLSKKIGIAHKIMGDQFAQTDQEQVGVENLWCGARMLSSDELAAATQGLVQESPLLSVNYPIGLIHPTTKENILSTQLLEKIAQSGLSHNEVFLVNTGDHWLLCLFYKLAEKIKCLIFNTYYDLNENTKQEIIEAAKIAGISESDEVNFIEMNLQNNVPNGCGLFCYHTIQLLSNAGQNDPATTLREFAENFLTLSVEEQALFNTQTRRQIYEYSLQ

SEQ ID NO: 66 - protein, 457 aa, unidentified
MPFTFQIGNHSCQISERYLRDIIDNKREHVFSTCEKFIDFFRNIFTRRSLISDYREIYNLLCQKKEHPDIKGPFSPGPFSKRDEDCTRWRPLLGYIKLIDASRPETIDKYTVEVLAHQENMLLLQMFYDGVLVTETECSERCVDFLKETMFNYNNGEITLAALGNDNLPPSEAGSNGIYEAFEQRLIDFLTTPATASGYESGAIDQTDASQPAAIEAFINSPEFQKNIRMRDIEKNKIGSGSYGTVYRLHDDFVVKIPVNERGIKVDVNSPEHRNCHPDRVSKYLNMANDDKNFSRSAIMNINGKDVTVLVSKYIQGQEFDVEDEDNYRMAEALLKSRGVYMHDINILGNILVKEGVLFFVDGDQIVLSQESRQQRSVSLATRQLEEQIKAHHMIKLKRAETEGNTEDVEYYKSLITDLDALIGEEEQTPAPGRRFKLAAPEEGTLVAKVLKDELKK

SEQ ID NO: 67 - protein, 700 aa, unidentified
MFNIRNTQPSVSMQAIAGAAAPEASPEEIVWEKIQVFFPQENYEEAQQCLAELCHPARGMLPDHISSQFARLKALTFPAWEENIQCNRDGINQFCILDAGSKEILSITLDDAGNYTVNCQGYSEAHDFIMDTEPGEECTEFAEGASGTSLRPATTVSQKAAEYDAVWSKWERDAPAGESPGRAAVVQEMRDCLNNGNPVLNVGASGLTTLPDRLPPHITTLVIPDNNLTSLPELPEGLRELEVSGNLQLTSLPSLPQGLQKLWAYNNWLASLPTLPPGLGDLAVSNNQLTSLPEMPPALRELRVSGNNLTSLPALPSGLQKLWAYNNRLTSLPEMSPGLQELDVSHNQLTRLPQSLTGLSSAARVYLDGNPLSVRTLQALRDIIGHSGIRIHFDMAGPSVPREARALHLAVADWLTSAREGEAAQADRWQAFGLEDNAAAFSLVLDRLRETENFKKDAGFKAQISSWLTQLAEDAALRAKTFAMATEATSTCEDRVTHALHQMNNVQLVHNAEKGEYDNNLQGLVSTGREMFRLATLEQIAREKAGTLALVDDVEVYLAFQNKLKESLELTSVTSEMRFFDVSGVTVSDLQAAELQVKTAENSGFSKWILQWGPLHSVLERKVPERFNALREKQISDYEDTYRKLYDEVLKSSGLVDDTDAERTIGVSAMDSAKKEFLDGLRALVDEVLGSYLTARWRLN

SEQ ID NO: 68 - protein, 788 aa, unidentified
MPFHIGSGCLPATISNRRIYRIAWSDTPPEMSSWEKMKEFFCSTHQTEALECIWTICHPPAGTTREDVINRFELLRTLAYAGWEESIHSGQHGENYFCILDEDSQEILSVTLDDAGNYTVNCQGYSETHRLTLDTAQGEEGTGHAEGASGTFRTSFLPATTAPQTPAEYDAVWSAWRRAAPAEESRGRAAVVQKMRACLNNGNAVLNVGESGLTTLPDCLPAHITTLVIPDNNLTSLPALPPELRTLEVSGNQLTSLPVLPPGLLELSIFSNPLTHLPALPSGLCKLWIFGNQLTSLPVLPPGLQELSVSDNQLASLPALPSELCKLWAYNNQLTSLPMLPSGLQELSVSDNQLASLPTLPSELYKLWAYNNRLTSLPALPSGLKELIVSGNRLTSLPVLPSELKELMVSGNRLTSLPMLPSGLLSLSVYRNQLTRLPESLIHLSSETTVNLEGNPLSERTLQALREITSAPGYSGPIIRFDMAGASAPRETRALHLAAADWLVPAREGEPAPADRWHMFGQEDNADAFSLFLDRLSETENFIKDAGFKAQISSWLAQLAEDEALRANTFAMATEATSSCEDRVTFFLHQMKNVQLVHNAEKGQYDNDLAALVATGREMFRLGKLEQIAREKVRTLALVDEIEVWLAYQNKLKKSLGLTSVTSEMRFFDVSGVTVTDLQDAELQVKAAEKSEFREWILQWGPLHRVLERKAPERVNALREKQISDYEETYRMLSDTELRPSGLVGNTDAERTIGARAMESAKKTFLDGLRPLVEEMLGSYLNVQWRRN

SEQ ID NO: 69 - protein, 385 aa, unidentified
MMASRDKDADSLIMYDRTLLWLTFGLAAIGFVMVTSASMPVGQRLANDPFLFAKRDALYIFLAFCLAMVTLRLPMTFWQKYSTTMLIASIIMLLIVLVVGSSVNGASRWIALGPLRIQPAEFTKLSLFCYLANYLVRKVDEVRNNLRGFLKPMGVILVLAVLLLAQPDLGTVVVLFVTTLAMLFLAGAKLWQFIAIIGMGISAVILLILAEPYRIRRVTSFWNPWEDPFGSGYQLTQSLMAFGRGEIWGQGLGNSVQKLEYLPEAHTDFIFAIIGEELGYIGVVLALLMVFFVAFRAMSIGRKALEIDHRFSGFLACSIGIWFSFQALVNVGAAAGMLPTKGLTLPLISYGGSSLLIMSTAIMFLLRIDYETRLEKAQAFTRGSR

SEQ ID NO: 70 - protein, 420 aa, unidentified
MIKATDRKLVVGLEIGTAKVAALVGEVLPDGMVNIIGVGSCPSRGMDKGGVNDLESVVKCVQRAIDQAELMADCQISSVYLALSGKHISCQNEIGMVPISEEEVTQEDVENVVHTAKSVRVRDEHRVLHVIPQEYAIDYQEGIKNPVGLSGVRMQAKVHLITCHNDMAKNIVKAVERCGLKVDQLIFAGLAASYSVLTEDERELGVCVVDIGGGTMDIAVYTGGALRHTKVIPYAGNVVTSDIAYAFGTPPSDAEAIKVRHGCALGSIVGKDESVEVPSVGGRPPRSLQRQTLAEVIEPRYTELLNLVNEEILQLQEQLRQQGVKHHLAAGIVLTGGAAQIEGLAACAQRVFHTQVRIGAPLNITGLTDYAQEPYYSTAVGLLHYGKESHLNGEAEVEKRVTASVGSWIKRLNSWLRKEF

SEQ ID NO: 71 - protein, 383 aa, unidentified
MFEPMELTNDAVIKVIGVGGGGGNAVEHMVRERIEGVEFFAVNTDAQALRKTAVGQTIQIGSGITKGLGAGANPEVGRNAADEDREALRAALEGADMVFIAAGMGGGTGTGAAPVVAEVAKDLGILTVAVVTKPFNFEGKKRMAFAEQGITELSKHVDSLITIPNDKLLKVLGRGISLLDAFGAANDVLKGAVQGIAELITRPGLMNVDFADVRTVMSEMGYAMMGSGVASGEDRAEEAAEMAISSPLLEDIDLSGARGVLVNITAGFDLRLDEFETVGNTIRAFASDNATVVIGTSLDPDMNDELRVTVVATGIGMDKRPEITLVTNKQVQQPVLDRYQQHGMAPLTQEQKTVAKVVNDNTPQAAKEPDYLDIPAFLRKQAD

SEQ ID NO: 72 - protein, 495 aa, unidentified
MADRNLRDLLAPWVAGLPARELREMTLDSRVAAAGDLFVAVVGHQADGRRYIPQAIAQGVAAIIAEAKDEASDGEIREMHGVPVVYLSQLNERLSALAGRFYHEPSENMRLVAVTGTNGKTTTTQLLAQWSQLLGETSAVMGTVGNGLLGKVIPTENTTGSAVDVQHVLASLVAQGATFGAMEVSSHGLVQHRVAALKFAASVFTNLSRDHLDYHGDMAHYEAAKWMLYSTHHHGQAIVNADDEVGRRWLASLPDAVAVSMEGHINPNCHGRWLKAEAVEYHDRGATIRFASSWGEGEIESRLMGAFNVSNLLLALATLLALGYPLTDLLKTAARLQPVCGRMEVFTAPGKPTVVVDYAHTPDALEKALQAARLHCAGKLWCVFGCGGDRDKGKRPLMGAIAEEFADIVVVTDDNPRTEEPRAIINDILAGMLDAGQVRVMEGRAEAVTNAIMQAKDNDVVLIAGKGHEDYQIVGTQRLDYSDRVTAARLLGVIA

SEQ ID NO: 73 - protein, 440 aa, unidentified
MSEFSQTVPELVAWARKNDFSISLPVDRLSFLLAVATLNGERLDGEMSEGELVDAFRHVSDAFEQTSETIGVRANNAINDMVRQRLLNRFTSEQAEGNAIYRLTPLGIGITDYYIRQREFSTLRLSMQLSIVAGELKRAADAAAEGGDEFHWHRNVYAPLKYSVAEIFDSIDLTQRIMDEQQQQVKDDIAQLLNKDWRAAISSCELLLSETSGTLRELQDTLEAAGDKLQANLLRIQDATMTHDDLHFVDRLVFDLQSKLDRIISWGQQSIDLWIGYDRHVHKFIRTAIDMDKNRVFAQRLRQSVQTYFDDPWALTYANADRLLDMRDEEMALRDDEVTGELPPDLEYEEFNEIREQLAAIIEEQLAIYKTRQTPLDLGLVVREYLAQYPRARHFDVARIVIDQAVRLGVAQADFTGLPAKWQPINDYGAKVQAHVIDKY

SEQ ID NO: 74 - protein, 786 aa, unidentified
MKKRIPTLLATMIASALYSHQGLAADLASQCMLGVPSYDRPLVKGDTNDLPVTINADNAKGNYPDDAVFTGNVDIMQGNSRLQADEVQLHQKQAEGQPEPVRTVDALGNVHYDDNQVILKGPKGWANLNTKDTNVWEGDYQMVGRQGRGKADLMKQRGENRYTILENGSFTSCLPGSDTWSVVGSEVIHDREEQVAEIWNARFKVGPVPIFYSPYLQLPVGDKRRSGFLIPNAKYTTKNYFEFYLPYYWNIAPNMDATITPHYMHRRGNIMWENEFRYLTQAGEGVMELDYLPSDKVYEDDHPKEGDKHRWLFNWQHSGVMDQVWRFNVDYTKVSDSSYFNDFDSKYGSSTDGYATQKFSVGYAVQNFDATVSTKQFQVFNDQNTSSYSAEPQLDVNYYHNDLGPFDTRIYGQAVHFVNTKDNMPEATRVHLEPTINLPLSNRWGSLNTEAKLMATHYQQTNLDSYNSDPNNKNKLEDSVNRVMPQFKVDGKLIFERDMAMLAPGYTQTLEPRVQYLYVPYRDQSGIYNYDSSLLQSDYNGLFRDRTYGGLDRIASANQVTTGVTTRIYDDAAVERFNVSVGQIYYFTESRTGDDNIKWENDDKTGSLVWAGDTYWRISERWGLRSGVQYDTRLDSVATSSSSLEYRRDQDRLVQLNYRYASPEYIQATLPSYYSTAEQYKNGINQVGAVASWPIADRWSIVGAYYFDTNSSKPADQMLGLQYNSCCYAIRVGYERKLNGWDNDKQHAIYDNAIGFNIELRGLSSNYGLGTQEMLRSNILPYQSSM

SEQ ID NO: 75 - protein, 323 aa, unidentified
MAQEYTVEQLNHGRKVYDFMRWDFWAFGISGLLLIAAIVIMGVRGFNWGLDFTGGTVIEITLEKPAEMDVMREALQKAGYEEPQLQNFGSSHDIMVRMPPTEGETGGQVLGSKVVTIINEATNQNAAVKRIEFVGPSVGADLAQTGAMALLVALISILVYVGFRFEWRLAAGVVIALAHDVIITLGILSLFHIEIDLTIVASLMSVIGYSLNDSIVVSDRIRENFRKIRRGTPYEIFNVSLTQTLHRTLITSGTTLVVILMLYLFGGPVLEGFSLTMLIGVSIGTASSIYVASALALKLGMKREHMLQQKVEKEGADQPSILP

SEQ ID NO: 76 - protein, 432 aa, unidentified
MSKIVKVIGREIIDSRGNPTVEAEVHLEGGFVGMAAAPSGASTGSREALELRDGDKSRFLGKGVTKAVGAVNGPIAQAILGKDAKDQAGIDKIMIDLDGTENKSNFGANAILAVSLANAKAAAAAKGMPLYEHIAELNGTPGKYSMPVPMMNIINGGEHADNNVDIQEFMIQPVGAKTVKEAIRMGSEVFHHLAKVLKGKGMNTAVGDEGGYAPNLGSNAEALAVIAEAVKAAGYELGKDITLAMDCAASEFYKDGKYVLAGEGNKAFTSEEFTHFLEELTKQYPIVSIEDGLDESDWDGFAYQTKVLGDKIQLVGDDLFVTNTKILKEGIEKGIANSILIKFNQIGSLTETLAAIKMAKDAGYTAVISHRSGETEDATIADLAVGTAAGQIKTGSMSRSDRVAKYNQLIRIEEALGEKAPYNGRKEIKGQA

SEQ ID NO: 77 - protein, 320 aa, unidentified
MRQTKTGILLANLGTPDAPTPEAVKRYLKQFLSDRRVVDTPRLLWWPLLRGVILPLRSPRVAKLYQSIWMDGGSPLMVYSREQQQALAARLPDTPVALGMSYGSPSLESAVDELLASDVDHIVVLPLYPQYSCSTVGAVWDELGRILARKRRIPGISFIRDYADDGAYIDALAKSARESFARHGEPDVLLLSYHGIPQRYADEGDDYPQRCRDTTRELVSALGLPPEKVMMTFQSRFGREPWLTPYTDETLKMLGEKGTGHIQVMCPGFAADCLETLEEIAEQNREIFLEAGGKKYAYIPALNATPEHIDMMLKLTAPYR

SEQ ID NO: 78 - protein, 213 aa, unidentified
MGSNYIVIEGLEGAGKTTARDVVVETLEQLGIRNMIFTREPGGTQLAEKLRSLVLDIRSVGDEVITDKAEVLMFYAARVQLVETVIKPALAQGVWVIGDRHDLSTQAYQGGGRGIDQTMLATLRDAVLGDFRPDLTLYLDVTPEVGLKRARARGDLDRIEQESFDFFNRTRARYLELAAQDSRIRTIDATQPLDAVMRDIRATVTKWVQEQAA

SEQ ID NO: 79 - protein, 620 aa, unidentified
MSFDIAKYPTLALVDSTQELRLLPKESLPKLCDELRRYLLDSVSRSSGHFASGLGTVELTVALHYVYNTPFDQLIWDVGHQAYPHKILTGRRDKIGTIRQKGGLHPFPWRGESEYDVLSVGHSSTSISAGIGIAVAAEKEGKDRRTVCVIGDGAITAGMAFEAMNHAGDIRPDMLVILNDNEMSISENVGALNNHLAQLLSGKLYSSLREGGKKVFSGVPPIKELLKRTEEHIKGMVVPGTLFEELGFNYIGPVDGHDVMGLISTLKNMRDLKGPQFLHIMTKKGRGYEPAEKDPITFHAVPKFDPSSGCLPKSSGGLPGYSKIFGDWLCETAAKDSKLMAITPAMREGSGMVEFSRKFPDRYFDVAIAEQHAVTFAAGLAIGGYKPVVAIYSTFLQRAYDQVIHDVAIQKLPVMFAIDRAGIVGADGQTHQGAFDLSYLRCIPDMVIMTPSDENECRQMLFTGYHYNDGPTAVRYPRGNAQGVALTPLEKLPIGKGLVKRHGEKLAILNFGTLMPEAAKVAEALNATLVDMRFVKPLDDTLILEMAAQHDALVTLEENAIMGGAGSGVNEVLMAHRKPVPVLNIGLPDFFIPQGTQEEARAELGLDAAGIEAKIKAWLA

SEQ ID NO: 80 - protein, 252 aa, unidentified
MLSATQPVSENLPAHGCRHVAIIMDGNGRWAKKQGKIRAFGHKAGAKSVRRAVSFAANNGIDALTLYAFSSENWNRPAQEVSALMELFVWALDSEVKSLHRHNVRLRIIGDISRFNSRLQERIRKSEALTAHNTGLTLNIAANYGGRWDIVQGVRQLAEQVQAGVLRPDQIDEERLGQQICMHELAPVDLVIRTGGEHRISNFLLWQIAYAELYFTDVLWPDFDEQDFEGALHAFANRERRFGGTEPGDDKA

SEQ ID NO: 81 - protein, 285 aa, unidentified
MLKYRLISAFVLIPAVIAALFLLPPVGFAIITLVVCMLAAWEWGQLSGFAARSQRVWLAVLCGLLLALMLFLLPEYHHNIRQPLVEMSLWASLGWWVVALLLVLFYPGSAAIWRNSKTLRLIFGLLTIVPFFWGMLALRAWHYDENHYSGAIWLLYVMILVWGADSGAYMFGKLFGKHKLAPKVSPGKTWQGFIGGLATAAVISWGYGMWANLNVAPVILLICSVVAALASVLGDLTESMFKREAGIKDSGHLIPGHGGILDRIDSLTAAVPVFACLLLLVFRTL

SEQ ID NO: 82 - protein, 319 aa, unidentified
MSLNFLDFEQPIAELEAKIDSLTAVSRQDEKLDINIDEEVHRLREKSVELTRKIFADLGAWQVAQLARHPQRPYTLDYVRLAFDEFDELAGDRAYADDKAIVGGIARLEGRPVMIIGHQKGRETKEKIRRNFGMPAPEGYRKALRLMEMAERFNMPIITFIDTPGAYPGVGAEERGQSEAIARNLREMSRLNVPVICTVIGEGGSGGALAIGVGDKVNMLQYSTYSVISPEGCASILWKSADKAPLAAEAMGIIAPRLKELKLIDSIIPEPLGGAHRNPEAMAASLKAQLLEDLADLDVLSTDDLKNRRYQRLMSYGYA

SEQ ID NO: 83 - protein, 451 aa, unidentified
MLSKFKRNKHQQHLAQLPKISQSVDDVDFFYTPATFRETLLEKIASATQRICIVALYLEQDDGGKGILDALYAAKRQRPELDVRVLVDWHRAQRGRIGAAASNTNADWYCRLAQENPGIDVPVYGVPINTREALGVLHFKGFIIDDSVLYSGASLNDVYLHQHDKYRYDRYQLIRNRQMADIMFDWVTQNLMNGRGVNRLDNTQRPKSPEIKNDIRLYRQELRDASYHFQGDANDEQLSVTPLVGLGKSSLLNKTIFHLMPCAEHKLTICTPYFNLPAVLVRNIIQLLRDGKKVEIIVGDKTANDFYIPEDEPFKIIGALPYLYEINLRRFLSRLQYYVNTDQLVVRLWKDDDNTYHLKGMWVDDKWMLLTGNNLNPRAWRLDLENAILIHDPKQELAPQREKELELIRTHTTIVKHYRDLQSIADYPIKVRKLIRRLRRIRIDRLISRIL

SEQ ID NO: 84 - protein, 582 aa, unidentified
MHNDKDLSTWQTFRRLWPTIAPFKAGLIVAGIALILNAASDTFMLSLLKPLLDDGFGKTDRSVLLWMPLVVIGLMILRGITSYISSYCISWVSGKVVMTMRRRLFGHMMGMPVAFFDKQSTGTLLSRITYDSEQVASSSSGALITVVREGASIIGLFIMMFYYSWQLSIILVVLAPIVSIAIRVVSKRFRSISKNMQNTMGQVTTSAEQMLKGHKEVLIFGGQEVETKRFDKVSNKMRLQGMKMVSASSISDPIIQLIASLALAFVLYAASFPSVMDSLTAGTITVVFSSMIALMRPLKSLTNVNAQFQRGMAACQTLFAILDSEQEKDEGKRVIDRATGDLEFRNVTFTYPGREVPALRNINLKIPAGKTVALVGRSGSGKSTIASLITRFYDIDEGHILMDGHDLREYTLASLRNQVALVSQNVHLFNDTVANNIAYARTEEYSREQIEEAARMAYAMDFINKMDNGLDTIIGENGVLLSGGQRQRIAIARALLRDSPILILDEATSALDTESERAIQAALDELQKNRTSLVIAHRLSTIEQADEIVVVEDGIIVERGTHSELLAQHGVYAQLHKMQFGQ

SEQ ID NO: 85 - protein, 283 aa, unidentified
MAEITASLVKELRERTGAGMMDCKKALTEANGDIELAIENMRKSGAIKAAKKAGNVAADGVIKTKIDGNVAFILEVNCQTDFVAKDAGFQAFADKVLDAAVAGKITDVEVLKAQFEEERVALVAKIGENINIRRVASLEGDVLGSYQHGARIGVLVAAKGADEELVKQLAMHVAASKPEFVKPEDVSADVVEKEYQVQLDIAMQSGKPKEIAEKMVEGRMKKFTGEVSLTGQPFVMEPSKSVGQLLKEHNADVTGFIRFEVGEGIEKVETDFAAEVAAMSKQS

SEQ ID NO: 86 - protein, 255 aa, unidentified
MFIGIVSLFPEMFRAITDYGVTGRAVKKGLLNIQSWSPRDFAHDRHRTVDDRPYGGGPGMLMMVQPLRDAIHAAKAAAGEGAKVIYLSPQGRKLDQAGVSELATNQKLILVCGRYEGVDERVIQTEIDEEWSIGDYVLSGGELPAMTLIDSVARFIPGVLGHEASAIEDSFADGLLDCPHYTRPEVLEGMEVPPVLLSGNHAEIRRWRLKQSLGRTWLRRPELLENLALTEEQARLLAEFKTEHAQQQHKHDGMA

SEQ ID NO: 87 - protein, 413 aa, unidentified
MKIYLVGGAVRDALLGLPVKDKDWVVVGATPQEMLDAGYQQVGRDFPVFLHPQTHEEYALARTERKSGSGYTGFTCYAAPDVTLEADLQRRDLTINALARDDAGQIIDPYHGRRDLEARLLRHVSPAFGEDPLRVLRVARFAARYAHLSFRIADETLALMREMTAAGELEHLTPERVWKETENALTTRNPQVYFQVLRDCGALRVLFPEIDALFGVPAPAKWHPEIDTGVHTLMTLSMAAMLSPQLDVRFATLCHDLGKGLTPKNLWPRHHGHGPAGVKLVEQLCQRLRVPNDLRDLAKLVAEYHDLIHTFPILQPKTIVKLFDAIDAWRKPQRVEQIALTSEADVRGRTGFEASDYPQGRWLREAWQVAQAVPTKEVVEAGFKGIEIREELTKRRIAAVANWKEKRCPNPAS

SEQ ID NO: 88 - protein, 892 aa, unidentified
MTDVTLKALAAERQVSVDRLVQQFADAGIRKSADDSVSAQEKQTLLAHLNREAVSGPDKLTLQRKTRSTLNIPGTGGKSKSVQIEVRKKRTFVKRDPQEAERLAAEEQAQREAEEQARREAEEQAKREAQQKAEREAAEQAKREAAEKAKREAAEKDKVSNQQTDDMTKTAQAEKARRENEAAELKRKAEEEARRKLEEEARRVAEEARRMAEENKWTATPEPVEDTSDYHVTTSQHARQAEDENDREVEGGRGRGRNAKAARPAKKGKHAESKADREEARAAVRGGKGGKRKGSSLQQGFQKPAQAVNRDVVIGETITVGELANKMAVKGSQVIKAMMKLGAMATINQVIDQETAQLVAEEMGHKVILRRENELEEAVMSDRDTGAAAEPRAPVVTIMGHVDHGKTSLLDYIRSTKVASGEAGGITQHIGAYHVETDNGMITFLDTPGHAAFTSMRARGAQATDIVVLVVAADDGVMPQTIEAIQHAKAAGVPVVVAVNKIDKPEADPDRVKNELSQYGILPEEWGGESQFVHVSAKAGTGIDELLDAILLQAEVLELKAVRKGMASGAVIESFLDKGRGPVATVLVREGTLHKGDIVLCGFEYGRVRAMRNELGQEVLEAGPSIPVEILGLSGVPAAGDEVTVVRDEKKAREVALYRQGKFREVKLARQQKSKLENMFANMTEGEVHEVNIVLKADVQGSVEAISDSLLKLSTDEVKVKIIGSGVGGITETDATLAAASNAILVGFNVRADASARKVIESESLDLRYYSVIYNLIDEVKAAMSGMLSPELKQQIIGLAEVRDVFKSPKFGAIAGCMVTEGTIKRHNPIRVLRDNVVIYEGELESLRRFKDDVNEVRNGMECGIGVKNYNDVRVGDMIEVFEIIEIQRTIA

SEQ ID NO: 89 - protein, 329 aa, unidentified [sequence omitted]

SEQ ID NO: 90 - protein, 1342 aa, unidentified
MVYSYTEKKRIRKDFGKRPQVLDVPYLLSIQLDSFQKFIEQDPEGQYGLEAAFRSVFPIQSYSGNSELQYVSYRLGEPVFDVQECQIRGVTYSAPLRVKLRLVIYEREAPEGTVKDIKEQEVYMGEIPLMTDNGTFVINGTERVIVSQLHRSPGVFFDSDKGKTHSSGKVLYNARIIPYRGSWLDFEFDPKDNLFVRIDRRRKLPATIILRALNYTTEQILDLFFEKVVFEIRDNKLQMELIPERLRGETASFDIEANGKVYVEKGRRITARHIRQLEKDDIKHIEVPVEYIAGKVVSKDYVDESTGELICAANMELSLDLLAKLSQSGHKRIETLFTNDLDHGPYISETVRVDPTNDRLSALVEIYRMMRPGEPPTREAAESLFENLFFSEDRYDLSAVGRMKFNRSLLRDEIEGSGILSKDDIIDVMKKLIDIRNGKGEVDDIDHLGNRRIRSVGEMAENQFRVGLVRVERAVKERLSLGDLDTLMPQDMINAKPISAAVKEFFGSSQLSQFMDQNNPLSEITHKRRISALGPGGLTRERAGFEVRDVHPTHYGRVCPIETPEGPNIGLINSLSVYAQTNEYGFLETPYRRVVDGVVTDEIHYLSAIEEGNYVIAQANSNLDDEGHFVEDLVTCRSKGESSLFSRDQVDYMDVSTQQVVSVGASLIPFLEHDDANRALMGANMQRQAVPTLRADKPLVGTGMERAVAVDSGVTAVAKRGGTVQYVDASRIVIKVNEDEMYPGEAGIDIYNLTKYTRSNQNTCINQMPCVSLGEPVERGDVLADGPSTDLGELALGQNMRVAFMPWNGYNFEDSILVSERVVQEDRFTTIHIQELACVSRDTKLGPEEITADIPNVGEAALSKLDESGIVYIGAEVTGGDILVGKVTPKGETQLTPEEKLLRAIFGEKASDVKDSSLRVPNGVSGTVIDVQVFTRDGVEKDKRALEIEEMQLKQAKKDLSEELQILEAGLFSRIRAVLVSSGVEAEKLDKLPRDRWLELGLTDEEKQNQLEQLAEQYDELKHEFEKKLEAKRRKITQGDDLAPGVLKIVKVYLAVKRRIQPGDKMAGRHGNKGVISKINPIEDMPYDENGTPVDIVLNPLGVPSRMNIGQILETHLGMAAKGIGDKINAMLKQQQEVAKLREFIQRAYDLGADVRQKVDLSTFSDDEVLRLAENLRKGMPIATPVFDGAKEAEIKELLKLGDLPTSGQITLFDGRTGEQFERPVTVGYMYMLKLNHLVDDKMHARSTGSYSLVTQQPLGGKAQFGGQRFGEMEVWALEAYGAAYTLQEMLTVKSDDVNGRTKMYKNIVDGNHQMEPGMPESFNVLLKEIRSLGINIELEDE

SEQ ID NO: 91 - protein, 1407 aa, unidentified
MKDLLKFLKAQTKTEEFDAIKIALASPDMIRSWSFGEVKKPETINYRTFKPERDGLFCARIFGPVKDYECLCGKYKRLKHRGVICEKCGVEVTQTKVRRERMGHIELASPTAHIWFLKSLPSRIGLLLDMPLRDIERVLYFESYVVIEGGMTNLERQQILTEEQYLDALEEFGDEFDAKMGAEAIQALLKSMDLEQECETLREELNETNSETKRKKLTKRIKLLEAFVQSGNKPEWMILTVLPVLPPDLRPLVPLDGGRFATSDLNDLYRRVINRNNRLKRLLDLAAPDIIVRNEKRMLQEAVDALLDNGRRGRAITGSNKRPLKSLADMIKGKQGRFRQNLLGKRVDYSGRSVITVGPYLRLHQCGLPKKMALELFKPFIYGKLELRGLATTIKAAKKMVEREEAVVWDILDEVIREHPVLLNRAPTLHRLGIQAFEPVLIEGKAIQLHPLVCAAYNADFDGDQMAVHVPLTLEAQLEARALMMSTNNILSPANGEPIIVPSQDVVLGLYYMTRDCVNAKGEGMVLTGPKEAERIYRAGLASLHARVKVRITEYEKDENGEFVAHTSLKDTTVGRAILWMIVPKGLPFSIVNQALGKKAISKMLNTCYRILGLKPTVIFADQTMYTGFAYAARSGASVGIDDMVIPEKKHEIISEAEAEVAEIQEQFQSGLVTAGERYNKVIDIWAAANDRVSKAMMDNLQTETVINRDGQEEQQVSFNSIYMMADSGARGSAAQIRQLAGMRGLMAKPDGSIIETPITANFREGLNVLQYFISTHGARKGLADTALKTANSGYLTRRLVDVAQDLVVTEDDCGTHEGILMTPVIEGGDVKEPLRDRVLGRVTAEDVLKPGTADILVPRNTLLHEQWCDLLEANSVDAVKVRSVVSCDTDFGVCAHCYGRDLARGHIINKGEAIGVIAAQSIGEPGTQLTMRTFHIGGAASRAAAESSIQVKNKGSIKLSNVKSVVNSSGKLVITSRNTELKLIDEFGRTKESYKVPYGAVMAKGDGEQVAGGETVANWDPHTMPVITEVSGFIRFTDMIDGQTITRQTDELTGLSSLVVLDSAERTTGGKDLRPALKIVDAQGNDVLIPGTDMPAQYFLPGKAIVQLEDGVQISSGDTLARIPQESGGTKDITGGLPRVADLFEARRPKEPAILAEIAGIVSFGKETKGKRRLVITPVDGSDPYEEMIPKWRQLNVFEGERVERGDVISDGPEAPHDILRLRGVHAVTRYIVNEVQDVYRLQGVKINDKHIEVIVRQMLRKATIESAGSSDFLEGEQVEYSRVKIANRELEANGKVGATFSRDLLGITKASLATESFISAASFQETTRVLTEAAVAGKRDELRGLKENVIVGRLIPAGTGYAYHQDRMRRRAAGEQPATPQVTAEDASASLAELLNAGLGGSDNE

SEQ ID NO: 92 - protein, 343 aa, unidentified
MIRLYPEQLRAQLNEGLRAAYLLLGNDPLLLQESQDAIRLAAASQGFEEHHAFTLDPSTDWGSLFSLCQAMSLFASRQTLVLQLPENGPNAAMNEQLATLSELLHDDLLLIVRGNKLTKAQENAAWYTALADRSVQVSCQTPEQAQLPRWVAARAKAQNLQLDDAANQLLCYCYEGNLLALAQALERLSLLWPDGKLTLPRVEQAVNDAAHFTPFHWVDALLMGKSKRALHILQQLRLEGSEPVILLRTLQRELLLLVNLKRQSAHTPLRALFDKHRVWQNRRPMIGDALQRLHPAQLRQAVQLLTRTEITLKQDYGQSVWADLEGLSLLLCHKALADVFIDG

SEQ ID NO: 93 - protein, 245 aa, unidentified
MKNVGDLMQRLQKMMPAHITPAFKTGEELLAWQKEQGEIRAAALARENRAMKMQRTFNRSGIRPLHQNCSFDNYRVECDGQMNALSKARQYVDEFDGNIASFVFSGKPGTGKNHLAAAICNELLLRGKSVLIITVADIMSAMKDTFSNRETSEEQLLNDLSNVDLLVIDEIGVQTESRYEKVIINQIVDRRSSSKRPTGMLTNSNMEEMTKMLGERVMDRMRLGNSLWVNFTWDSYRSRVTGKEY

SEQ ID NO: 94 - protein, 490 aa, unidentified
MVPVVALVGRPNVGKSTLFNRLTRTRDALVADFPGLTRDRKYGRAEVEGREFICIDTGGIDGTEDGVETRMAEQSLLAIEEADVVLFMVDARAGLMPADEAIAKHLRSREKPTFLVANKTDGLDPDQAVVDFYSLGLGEIYPIAASHGRGVLSLLEHVLLPWMDDVAPQEEVDEDAEYWAQFEAEQNGEEAPEDDFDPQSLPIKLAIVGRPNVGKSTLTNRILGEERVVVYDMPGTTRDSIYIPMERDEREYVLIDTAGVRKRGKITDAVEKFSVIKTLQAIEDANVVLLVIDAREGISDQDLSLLGFILNSGRSLVIVVNKWDGLSQEVKEQVKETLDFRLGFIDFARVHFISALHGSGVGNLFESVREAYDSSTRRVSTAMLTRIMTMAVEDHQPPLVRGRRVKLKYAHAGGYNPPIVVIHGNQVKDLPDSYKRYLMNYFRKSLEVMGTPIRIQFKEGENPYANKRNTLTPTQMRKRKRLMKHIKKSK

SEQ ID NO: 95 - protein, 199 aa, unidentified
MMSAPDIRHLPSDCGIEVAFAGRSNAGKSSALNTLTNQKSLARTSKTPGRTQLINLFEVVDGKRLVDLPGYGYAEVPEEMKRKWQRALGEYLEKRQSLQGLVVLMDIRHPLKDLDQQMIQWAVESNIQVLVLLTKADKLASGARKAQLNMVREAVLAFNGDVQVEAFSSLKKQGVDKLRQKLDSWFSELAPVEEIQDGE

SEQ ID NO: 96 - DNA, 1223 bp, unidentified
  source 1..1223: mol_type = other DNA
ttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaacc

SEQ ID NO: 97 - DNA, 318 bp, unidentified
  source 1..318: mol_type = other DNA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaacc

SEQ ID NO: 98 - DNA, 879 bp, unidentified
  source 1..879: mol_type = other DNA
atggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataa

SEQ ID NO: 99 - protein, 292 aa, unidentified
MAEAQNDPLLPGYSFNAHLVAGLTPIEANGYLDFFIDRPLGMKGYILNLTIRGQGVVKNQGREFVCRPGDILLFPPGEIHHYGRHPEAREWYHQWVYFRPRAYWHEWLNWPSIFANTGFFRPDEAHQPHFSDLFGQIINAGQGEGRYSELLAINLLEQLLLRRMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCEEKVNDVAVKLS

SEQ ID NO: 100 - DNA, 98 bp, synthetic construct
  source 1..98: mol_type = other DNA
ccgcatagttaagccagtatacatttacactttatgcttccggctcgtataataaaaaaaaaaaaaaggaggaaaaaaaatgggctccatcggtgcag

SEQ ID NO: 101 - DNA, 54 bp, synthetic construct
  source 1..54: mol_type = other DNA
ctacagatcctcttctgagatgagtttttgttcaggggaaacacatctgccaaa

SEQ ID NO: 102 - DNA, 42 bp, synthetic construct
  source 1..42: mol_type = other DNA
tcatctcagaagaggatctgtaactccgctatcgctacgtga

SEQ ID NO: 103 - DNA, 23 bp, synthetic construct
  source 1..23: mol_type = other DNA
tgtatactggcttaactatgcgg

(19) United States
(12) Patent Application Publication
(10) Pub. No.: US 2025/0195631 A1
(43) Pub. Date: Jun. 19, 2025

(54) ANTIGEN DELIVERING SALMONELLA FOR USE AS A TUMOR HOMING BEACON TO REFOCUS PREEXISTING, VACCINE GENERATED T CELLS TO COMBAT CANCER

(71) Applicant: University of Massachusetts, Boston, MA (US)

(72) Inventors: Neil S. Forbes, Hadley, MA (US); Vishnu Raman, Sunderland, MA (US)

(21) Appl. No.: 18/851,628

(22) PCT Filed: Mar. 28, 2023
(86) PCT No.: PCT/US2023/065051

§ 371 Date: Sep. 26, 2024

Related U.S. Application Data
(60) Provisional application No. 63/362,034, filed on Mar. 28, 2022.

(51) Int. Cl. A61K 39/00; A61P 35/00; C07K 14/77; C12N 15/74

(52) U.S. Cl. CPC A61K 39/0005; A61P 35/00; C07K 14/77; C12N 15/74; A61K 2039/522; A61K 2039/523; C12N 2800/101; C12N 2820/002

(57) ABSTRACT

To make an immunotherapy that is effective for a larger group of cancer patients, Salmonella have been genetically engineered to deliver proteins from prior vaccines into the cytoplasm of tumor cells.

### PRIORITY APPLICATION

This application is a U.S. national stage filing under 35 U.S.C. § 371 from International Application No. PCT/US2023/065051, filed on 28 Mar. 2023, and published as WO/2023/192869 A1 on 5 Oct. 20203, which claims the benefit of priority to U.S. Provisional Patent Application Ser. No. 63/362,034, filed Mar. 28, 2022, the content of which is incorporated herein by reference in its entirety.

### INCORPORATION BY REFERENCE OF SEQUENCE LISTING

A Sequence Listing is provided herewith as an xml file, “3724067US1.xml” created on Oct. 25, 2024, and having a size of 127,311 bytes. The content of the xml file is incorporated by reference herein in its entirety.

### BACKGROUND OF THE INVENTION

Cancer immunotherapy has become an effective way to induce durable remissions in patients with late stage and unresectable tumors. However, existing immunotherapeutic strategies only work on tumors with inherent characteristics that cannot be externally controlled. For instance, chimeric antigen receptor T cells (CAR-T) require tumors to express specific surface biomarkers (e.g., CD19 for B cells malignancies). These autologous, cell-based therapies are labor, time and resource intensive to manufacture due to the need to customize the therapy for each patient. Immune checkpoint blockade requires expression of surface checkpoint receptors (e.g., PD-L1/PD-1) on tumor cells in addition to high tumor mutational burdens. The presence or absence of these features is fixed and different for every tumor. As a result, these existing immunotherapeutic approaches cannot be implemented in all cancer patients. An immunotherapy that is less dependent on a patient's inherent tumor characteristics would create an additional therapeutic option for widespread use. An off-the-shelf treatment approach that engages autologous CD8 T cells is needed to enable widespread, efficient use in cancer patients.

### SUMMARY OF THE INVENTION

Immunotherapies are not effective for all tumor types and are almost ineffective in pancreatic cancer. To address this challenge, an immunotherapeutic platform was developed that delivers antigens directly into the cytoplasm of cancer cells in tumors. Intracellular delivering (ID) Salmonella were genetically engineered to autonomously lyse inside cells and release a protein payload. This method of intracellular delivery is unique to this platform and is required to trigger antigen-specific T cells. Most protein delivery mechanisms (e.g., nanoparticles, cell-penetrating peptides, and antibody drug conjugates) do not trigger immune cell responses because they deliver proteins to endosomes, where they are trafficked to the lysosome and degraded. It was shown that cytoplasmic delivery of an immunization antigen activated cytotoxic CD8 T cells, eliminated pancreatic tumors in immunized mice, and increased survival. In vaccinated mice, the therapy prevented tumor re-implantation, indicating that it established antitumor immunity. By refocusing pre-existing vaccine-induced immunity towards tumors, this strategy would not require ex vivo processing, unlike CAR-T and other cell-based therapies. As an off-the-shelf immunotherapy, this bacterial system is effective for a broad range of cancer patients.

Provided herein are bacteria, for example engineered Salmonella, designed to invade cancer cells, lyse and deliver protein intracellularly, wherein the protein is one that elicits an immune response, such as those proteins/antigens used in vaccines. For example, genetically engineered Salmonella colonize and deliver protein selectively within the cytosol of tumor cells in vivo. Cytosolic protein is antigen presented on MHC-I receptors to cytotoxic, CD8 T cells. Due to the widespread use of vaccines, many people have preexisting, memory CD8 T cells against various pathogenic proteins. These T cells can become quickly reactivated and cytotoxic towards cells displaying the pathogenic antigen on surface MHC-I receptors. Since most humans have preexisting, vaccine induced immunity to these pathogen-associated proteins, it is provided herein that engineered Salmonella can deliver the model protein, e.g., ovalbumin, inside tumor cells and repurpose endogenous, ovalbumin vaccine associated, CD8 T cells to combat cancer.

Provided here is a non-pathogenic bacterial cell expressing a vaccine derived (vaccine antigen), or an exogenous (e.g., exogenous to the bacteria and/or the organism in which this would be administered to) immunogenic, protein intracellularly, wherein the cell comprises a lysis gene or lysis cassette operably linked to an intracellularly induced Salmonella promoter.

In one aspect, the expressed protein is coded for by an expression plasmid. In one aspect, the protein is a vaccine antigen found in one or more of the following vaccines that to immunize against anthrax (AVA (BioThrax); cholera (Vaxchora), COVID-19 (Pfizer-BioNTech; Moderna; Johnson & Johnson's Janssen), diptheria (DTaP (Daptacel, Infanrix); Td (Tenivac, generic); DT (-generic-); Tdap (Adacel, Boostrix); DTaP-IPV (Kinrix, Quadracel); DTaP-HepB-IPV (Pediarix); DTaP-IPV/Hib (Pentacel)), hepatitis A (HepA (Havrix, Vaqta); HepA-HepB (Twinrix)), Hepatitis B (HepB (Engerix-B, Recombivax H B, Heplisav-B); DTaP-HepB-IPV (Pediarix); HepA-HepB (Twinrix)), Haemophilus influenzae type b (Hib) (Hib (ActHIB, PedvaxHIB, Hiberix); DTaP-IPV/Hib (Pentacel)), Human Papillomavirus (HPV) (HPV9 (Gardasil 9) (For scientific papers, the preferred abbreviation is 9vHPV)), Seasonal Influenza (Flu) (IIV* (Afluria, Fluad, Flublok, Flucelvax, FluLaval, Fluarix, Fluvirin, Fluzone, Fluzone High-Dose, Fluzone Intradermal; there are various acronyms for inactivated flu vaccines—IIV3, IIV4, RIV3, RIV4 and cclIV4; LAIV (FluMist)), Japanese Encephalitis (JE (Ixiaro)), Measles (MMR (M-M-R II); MMRV (ProQuad)), Meningococcal (MenACWY (Menactra, Menveo); MenB (Bexsero, Trumenba)), Mumps (MMR (M-M-R II); MMRV (ProQuad)), Pertussis (DTaP (Daptacel, Infanrix); Tdap (Adacel, Boostrix); DTaP-IPV (Kinrix, Quadracel); DTaP-HepB-IPV (Pediarix); DTaP-IPV/Hib (Pentacel)), Pneumococcal (PCV13 (Prevnar13); PPSV23 (Pneumovax 23)), Polio (Polio (Ipol); DTaP-IPV (Kinrix, Quadracel); DTaP-HepB-IPV (Pediarix); DTaP-IPV/Hib (Pentacel)), Rabies (Rabies (Imovax Rabies, RabAvert)), Rotavirus (RV1 (Rotarix); RV5 (RotaTeq)), Rubella (MMR (M-M-R II); MMRV (ProQuad)), Shingles (RZV (Shingrix)), Smallpox (Vaccinia (ACAM2000)), Tetanus (DTaP (Daptacel, Infanrix); Td (Tenivac, generic), DT (-generic-), Tdap (Adacel, Boostrix), DTaP-IPV (Kinrix, Quadracel), DTaP-HepB-IPV (Pediarix), DTaP-IPV/Hib (Pentacel)), Typhoid Fever (Typhoid Oral (Vivotif); Typhoid Polysaccharide (Typhim Vi)), Varicella (VAR (Varivax); MMRV (ProQuad), Covid-19 (Novavax or ImmunityBio) and/or Yellow Fever (YF (YF-Vax)).

In another aspect, the cell comprises inducible expression of flagella.

In one aspect, expression of SseJ has been reduced. In another aspect, the cell comprises a SseJ deletion.

In one aspect, the immunogenic protein is constitutively or inducibly expressed.

In another aspect, the bacterial cell is an intratumoral bacteria cell. In one aspect, the bacterial cell is a Clostridium, Bifidus, Escherichia coli or Salmonella cell. In one aspect, the bacterial cell is a Salmonella cell.

In one aspect, the lysis cassette is Lysin E from phage phiX174, the lysis cassette of phage iEPS5, or the lysis cassette from lambda phage.

In one aspect, the intracellularly induced Salmonella promoter is a promoter for one of the genes in Salmonella pathogenicity island 2 type III secretion system (SPI2-T3SS) selected from the group SpiC/SsaB, SseF, SseG, SseI, SseJ, SseK1, SseK2, SifA, SifB, PipB, PipB2, SopD2, GogB, SseL, SteC, SspH1, SspH2, or SirP. In one aspect, the cell does not comprise endogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL expression. In one aspect, the cell comprises an exogenous inducible promoter operably linked to an endogenous or exogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL gene. In one aspect, the exogenous inducible promoter is operably linked to the endogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL gene. In one aspect, the exogenous inducible promoter is operably linked to the exogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL gene. In another aspect, the exogenous inducible promoter comprises the arabinose inducible promoter PBAD (L-arabinose), LacI (IPTG), salR or nahR (acetyl salicylic acid (ASA)).

One aspect provides a composition comprising a population of bacterial cells described herein and a pharmaceutically acceptable carrier.

One aspect provides a method to selectively colonize a tumor and/or tumor associated cells comprising administering a population of the bacterial cells described herein to a subject in need thereof. In one aspect, the tumor associated cells are intratumoral immune cells or stromal cells within tumors.

One aspect provides a method to treat cancer comprising administering to subject in need thereof an effective amount of a population of the bacterial cells described herein so as to treat said cancer, wherein the subject has previously been exposed to the vaccine derived protein.

Another aspect provides a method of inhibiting tumor growth/proliferation or reducing the volume/size of a tumor comprising administering to subject in need thereof an effective amount of a population of the bacterial cells described herein, so as to suppress tumor growth or reduce the volume of the tumor, wherein the subject has previously been exposed to the vaccine derived protein or the exogenous immunogenic protein.

One aspect provides a method to treat, reduce formation/number or inhibit spread of metastases comprising administering to subject in need thereof an effective amount of a population of the bacterial cells described herein, so as to treat, reduce formation/number or inhibit spread of metastases, wherein the subject has previously been exposed to the vaccine derived protein or the exogenous immunogenic protein.

One aspect provides a method to treat cancer comprising administering an effective amount of a population of the bacterial cells described herein to a subject in need thereof, wherein the bacteria deliver vaccine-derived antigen in the cancer cells so as to elicit an anti-tumor, CD8 T cell specific immune response, wherein the subject has previously been exposed to the vaccine derived protein. In one aspect, the anti-tumor, CD8 T cell specific immune response is an anti-tumor, memory CD8T cell specific immune response.

Another aspect provides a method to treat cancer comprising administering an effective amount of a population of the bacterial cells described herein to a subject in need thereof, wherein the bacteria deliver antigen in the cancer cells so as to elicit an anti-tumor, CD4 T cell specific immune response, wherein the subject has previously been exposed to the vaccine derived protein. In one aspect, the anti-tumor, CD4 T cell specific immune response is an anti-tumor, memory CD4 T cell specific immune response.

One aspect provides a method to provide an anti-tumor, vaccine associated, CD8 T cell specific immune response comprising administering an effective amount of a population of the bacterial cells described herein to a subject in need thereof, wherein the subject has previously been exposed to the vaccine derived protein. In one aspect, the anti-tumor, CD8 T cell specific immune response is an anti-tumor, memory CD8T cell specific immune response.

One aspect provides a method to provide an anti-tumor, vaccine associated, CD4 T cell specific immune response comprising administering an effective amount of a population of the bacterial cells described herein to a subject in need thereof, wherein the subject has previously been exposed to the vaccine derived protein. In one aspect, the anti-tumor, CD4 T cell specific immune response is an anti-tumor, memory CD4 T cell specific immune response.

In one aspect of the methods disclosed herein, the bacterial cells deliver said vaccine derived peptide to said tumor, tumor associated cells, cancer, or metastases. In another aspect of the methods disclosed herein, the tumor, tumor associated cells, cancer, or metastases are a lung, liver, kidney, breast, prostate, pancreatic, colon, head and neck, ovarian and/or gastroenterological tumor, tumor associated cells, cancer or metastases.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1G. ID Salmonella deliver proteins into cells in tumors. A) Antigen delivery with intracellular delivering (ID) Salmonella. After ID Salmonella invade cancer cells (1), the bacteria autonomously lyse and deposit recombinant antigens into the cellular cytoplasm (2). Cytoplasmic antigens are presented on the cell surface (12, 17). B) Salmonella that intracellularly deliver GFP were created by transformation with a plasmid that contains circuits that produce the protein (Plac-GFP), control cell invasion (PBAD-flhDC), maintain the plasmid without antibiotics (Pasd-ASD), and cause lysis after cell invasion (PsseJ-LysE). Control Salmonella (bottom) were created that invade and produce GFP, but do not lyse. C) Lysing (ID) and non-lysing control Salmonella were administered to 4T1 cancer cells in culture (n=9). After cell invasion, GFP (green, white arrows, left) was released from intracellular ID Salmonella (red, black arrows, left), but was not released from non-lysing controls (red, black arrows, right). D) ID Salmonella delivered GFP to significantly more cells than non-lysing controls (P<0.0001). E) Intracellular delivery was measured in BALB/c mice implanted with 4T1 tumor cells. Once tumors reached 500 mm3 (about 14 days), mice were intravenously injected with lysing (ID-GFP) or non-lysing Salmonella. After 48 and 72 h, flhDC driven cell invasion was induced with IP injections of arabinose. At 96 h, tumors were harvested for histological examination. F) ID-GFP Salmonella invaded and intracellularly delivered GFP throughout the cytoplasm of cells within tumors (white arrows, left). Non-lysing Salmonella (red) invaded cancer cells but did not deliver GFP (right). G) Protein delivery was six times greater in cells containing ID-GFP Salmonella compared to non-lysing controls (P=0.0001; n=14 for non-lysing and n=12 for lysing). Data are shown as means≅SEM. Statistical comparison is a two-tailed, unpaired Student's t test with asterisks indicating significance (***, P<0.001). The scale bar in (C) is 10 μm.

FIGS. 2A-2F. FIG. 2. Bacterial delivery of ovalbumin induces a specific CD8 T cell response. A) Salmonella with a genomic flhD and ASD double knockout were transformed with an antigen delivery plasmid to create the ID-OVA strain. The plasmid contains four genetic circuits: (1) PBAD513-fthDC to control cell invasion, (2) Plac-OVA to produce ovalbumin constitutively, (3) PsseJ-LysE to induce autonomous intracellular lysis, and (4) Pasd-ASD for plasmid retention. B) After administration to 4T1 cancer cells, ID-OVA invaded the cells and delivered ovalbumin (green, arrows) throughout the cytoplasm. C) ID-OVA and ID-GFP were administered to Hepa 1-6 cells at a multiplicity of infection (MOI) of 20 (n=17). After cell invasion, ID-GFP and ID-OVA lysed and released their produced protein into the cellular cytoplasm (green, white arrows). Some intracellular bacteria did not lyse (black arrow). D) There was no significant difference in the fraction of cells with delivered protein. E) ID-OVA Salmonella were administered to Hepa 1-6 cancer cells to measure the effect of ovalbumin delivery on T cell cytotoxicity. ID-OVA were administered at a MOI of 10:1 for 2 b. CD8 T cells were isolated from the spleens of OT-I mice and were activated with anti-CD3° C. antibody, followed by IL-2 and anti-CD28 antibody. Immediately after bacterial clearance with gentamicin, the isolated T cells were co-cultured with the cancer cells at a ratio of 10:1 for 48 h. F) The activated CD8 T cells killed more cancer cell after administration of ID-OVA compared to ID-GFP (*, P=0.011; n=3). Measurements are arbitrary units, normalized by death due to cell culture and bacterial invasion. Data are shown as means±SEM. The statistical comparisons in (D) and (F) are two-tailed, unpaired Student's t tests. Asterisks indicate significance (*, P<0.05). The scale bar in (C) is 10 μm.

FIGS. 3A-3G. Bacterial delivery of ovalbumin induced an antigen-specific T cell response. A) To determine the effect of antigen delivery on tumor volume, ID-OVA Salmonella were administered to mice with adoptively transferred CD8 T cells from OT-I mice. To determine the dependence on T cells, bacteria were also administered to control mice that did not receive transferred T cells. For all treatment groups, MC38 tumor cells were injected into wild-type C57BL/6 mice. When tumors reached approximately 50 mm3, they were injected with either ID-GFP or ID-OVA. Two days after bacterial injection, OT-I T cells were intravenously injected into the adoptive transfer mice and tumor volumes were recorded twice a week. Arabinose (100 mg) was injected IP at 48 and 72 hours after bacterial injection to induce flhDC expression. B, C) The purity and activation of isolated OT-I T cells was determined by expression of CD8 (B) and co-expression of CD8 and CD44 (C). D) Mice with adoptively transferred OT-I CD8 T cells and administered ID-OVA had reduced tumor growth compared to mice administered ID-GFP (P=0.031 at 20 days; n=6). E) Individual tumor growth trajectories of mice administered with ID-GFP. F) Individual tumor growth trajectories of mice administered with ID-GFP. One mouse had a partial response (lower red line) and another had a complete response (upper red line). G) In mice without adoptive transfer, there was no difference in tumor response to ID-OVA and ID-GFP (n=8). Data are shown as means #SEM. Statistical comparison in (D) is a two-tailed, unpaired Student's t tests with asterisk indicating significance (*, P<0.05).

FIGS. 4A-4F. FIG. 4. Exogenous antigen delivery with ID Salmonella refocuses vaccine immunity against tumors. A) C57BL/6 mice were immunized against ovalbumin with two intraperitoneal injections of ovalbumin and poly(I:C), as an adjuvant, spaced seven days apart. Seven days after the second ovalbumin injection, the immunized mice were subcutaneously injected with 1×10⁵ MC38 tumor cells. Once tumors were between 50-75 mm3 (about two weeks), the mice were intratumorally injected with either ID-GFP or ID-OVA. The mice also received intraperitoneal injections of 50 μg of anti-PD-1 checkpoint blockade 48 h after bacterial injection. B) Ovalbumin immunized mice administered with ID-OVA had significantly slower tumor growth compared to control ID-GFP mice (P=0.044 at 12 days and P=0.049 at 18 days; n=8). C) By 18 days after bacterial administration, four of the eight mice administered ID-OVA had tumor volumes less than 110 mm³ (red lines). D) Comparatively, at the same time point, none of the mice injected with ID-GFP had tumors less than 250 mm³. E) The growth rate of responsive ID-OVA tumors was significantly lower than ID-GFP tumors (P=0.0012; n=8 for ID-GFP and n=4 for responsive and less-responsive ID-OVA). F) Administration of ID-OVA to ovalbumin-immunized mice significantly increased survival compared to control ID-GFP mice (*, P=0.0480). Data are shown as means #SEM. The statistical comparisons in (B), (E), and (F) are two-tailed, unpaired Student's t tests; ANOVA followed by Dunnett's method; and a log-rank test, respectively. Asterisks indicate significance (*, P<0.05; **, P<0.01).

FIGS. 5A-5G. ID-OVA cleared KPC pancreatic tumors and prevented tumor re-challenge. A) C57BL/6 mice were immunized with two intraperitoneal injections of ovalbumin and poly(I:C) spaced 28 days apart. Pancreatic tumors were initiated seven days after the second immunization with a subcutaneous injection of 2×10⁵ KPC PDAC cells. Once tumors were between 30-50 mm³, they were injected with (1) saline (n=8), (2) 50 mg/kg of gemcitabine (n=8), (3) 2×10⁷ CFU control ID-GFP Salmonella (n=7), or (4) 2×10⁷ CFU of ID-OVA Salmonella (n=7). These injections continued every five days until mice were removed from the study or tumors were too small to be detected (four injections for all mice). All mice received intraperitoneal injections of 400 mg of arabinose 48 and 72 hours after therapeutic administration. After treatment, tumor volume was measured every three days. Mice with completely cleared primary tumors were re-challenged with 1×105 KPC PDAC cells on the opposite flank 14 days after clearance and monitored for tumor regrowth for at least 14 days. B) Tumor volume as a function of time. From day 7 to 19, tumors from mice injected with ID-OVA were significantly smaller than saline controls (d 7, P=0.0052; d 10, P=0.00016; d 13, P=0.0031; d 16, P<0.0001; d 19, P<0.0001). C) Treatment with ID-OVA significantly reduced the growth rate of KPC PDAC tumors (P=0.0004). D) Three mice treated with ID-OVA had complete responses and the remaining four had partial responses. Between days 10 and 16, the tumors in mice with partial responses were significantly smaller than saline controls (d 10, P=0.0075; d 13, P=0.036; d 16, P=0.0046). E) Treatment with ID-OVA increased survival compared to saline (P=0.0012) and gemcitabine (P=0.026). F) After treatment with ID-OVA, the volume of tumors (red lines) of three mice completely cleared (left axis). Two weeks after clearance, mice were injected with KPC PDAC cells on the opposite flank. No new tumors appeared. For comparison, tumor volumes of naïve controls injected with KPC PDAC cells (right axis) are shown, aligned at the same injection time. G) The growth rates of re-implanted tumors were significantly less than naïve controls (P<0.0001). Data are shown as means±SEM. Statistical comparisons in (B) and (D) are ANOVA with Bonferroni correction; in (C) are ANOVA followed by Dunnett's multiple comparisons test; in (E) are log-rank tests with Bonferroni correction; and in (G) are two-tailed, unpaired Student's t tests. Asterisks indicate significance (*, P<0.05; **, P<0.01; ***, P<0.001; ****, P<0.0001).

FIG. 6. Mechanism of acquired antitumor immunity from intracellular bacterial antigen delivery. (1) Salmonella invade into cancer cells, and (2) autonomously lyse releasing bacterially expressed antigens (orange) into the cytoplasm. (3) Presentation of the delivered antigen activates antigen-specific vaccine CD8 T cells (12, 20), which kill the presenting cancer cells (62-66). (4) Cancer cell death and T cell activation induce antigen presenting cells (APCs) to cross-present tumor associated antigens (TAAs, brown) (55-58). (5) Activation of tumor-specific CD8 T cells (23, 24, 67-69) leads to the formation of antitumor immunity (70-73).

### DETAILED DESCRIPTION

For the purposes of clarity and a concise description, features can be described herein as part of the same or separate embodiments; however, it will be appreciated that the scope of the invention may include embodiments having combinations of all or some of the features described.

Provided herein is an off-the-shelf immunotherapeutic strategy to engage previously existing, vaccine generated immune cells to target cancer. Engineered bacteria, such as Salmonella, selectively colonize and deliver protein into tumor cells (see for example, U.S. Provisional Application Ser. No. 63/147,506, which is incorporated in its entirety herein by reference). Using this knowledge, herein is a bacterial delivery technology to refocus preexisting, vaccine generated immune cells to combat cancer. Since vaccines are widely administered (70% of people are vaccinated against 9 different pathogens hcdc.gov/nchs/fastats/immunize.htm), this delivery system can serve as an off-the-shelf, autologous immune cell (predominantly CD8 and CD4 T cells) therapy to combat cancer.

Existing T cell cancer immunotherapies are effective but cannot be utilized in a cost effective, rapidly deployable and off-the-shelf manner. Existing CD8 T cell cancer therapies require the T cells to be harvested from a cancer patient's own blood, genetically engineered, expanded and reinfused back into the patient. This process is expensive and many times; cannot be performed in time to save a patient.

The technology provided herein circumvents the need to create CD8 T cell therapies in a patient specific manner. The ability of engineered bacteria, such as Salmonella, to deliver vaccine-associated proteins inside cancer cells functions as a safe, rapidly deployable and off-the-shelf method to treat cancer. This novel delivery method does not need to be customized for each patient. The bacterial, e.g., Salmonella, based antigen delivery system could refocus a patient's own T cells as long as they have been vaccinated against the same delivered antigen.

Demonstrated herein is that engineered Salmonella can deliver ovalbumin into the cytosol of cancer cells. The engineered Salmonella was administered into tumor bearing mice containing activated, adoptively transferred, OT-I T cells. These mice exhibited slower tumor growth compared to a control. One of these mice achieved a partial response while another achieved a complete response. Finally, the ovalbumin expressing, engineered Salmonella were administered to tumor bearing mice previously vaccinated against ovalbumin. The tumor bearing mice receiving ovalbumin delivering Salmonella exhibited reduced tumor growth compared to control. These results demonstrate that Salmonella could deliver vaccine antigen into tumor cells and refocus vaccine associated CD8 T cells to target cancer. Every vaccinated cancer patient already harbors primed immune cells from vaccines that do not need to be processed ex vivo. Repurposing these endogenous, preexisting immune cells to fight cancer with tumor selective Salmonella would create a technology that is inexpensive and rapidly scalable for use in any vaccinated cancer patient.

Demonstrated herein is engineered Salmonella that can selectively deliver vaccine associated antigen into the cytosol of tumor cells and refocus vaccine derived immune cells (including CD8 T cells) to target cancer. Vaccine antigen delivery selectively into tumor cells with engineered Salmonella presents a novel, off-the-shelf, method to engage preexisting/endogenous, vaccine derived T cells to combat cancer.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments; however, it will be appreciated that the scope of the invention may include embodiments having combinations of all or some of the features described.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The terms “individual,” “subject,” and “patient,” are used interchangeably herein and refer to any subject for whom diagnosis, treatment, or therapy is desired, including a mammal. Mammals include, but are not limited to, humans, farm animals, sport animals and pets. A “subject” is a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, orangutan) rat, sheep, goat, cow and bird.

The terms “treatment”, “treating” and the like are used herein to generally mean obtaining a desired pharmacologic and/or physiologic effect, such as arresting or inhibiting, or attempting to arrest or inhibit, the development or progression of a disorder and/or causing, or attempting to cause, the reduction, suppression, regression, or remission of a disorder and/or a symptom thereof. The effect may be prophylactic in terms of completely or partially preventing a disease or symptom thereof and/or may be therapeutic in terms of a partial or complete cure for a disease and/or adverse effect attributable to the disease. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms. Additionally, treatment can be applied to a subject or to a cell culture (in vivo or in vitro).

The terms “inhibit”, “inhibiting”, and “inhibition” refer to the slowing, halting, or reversing the growth or progression of a disease, infection, condition, group of cells, protein or its expression. The inhibition can be greater than about 20%, 40%, 60%, 80%, 90%, 95%, or 99%, for example, compared to the growth or progression that occurs in the absence of the treatment or contacting.

“Expression” refers to the production of RNA from DNA and/or the production of protein directed by genetic material (e.g., RNA (mRNA)). Inducible expression, as opposed to constitutive expression (expressed all the time), is expression which only occurs under certain conditions, such as in the presence of specific molecule (e.g., arabinose) or an environmental que.

The term “exogenous” as used herein with reference to a nucleic acid (or a protein) and a host refers to a nucleic acid that does not occur in (and cannot be obtained from) a cell of that particular type as it is found in nature, or a protein encoded by such a nucleic acid. Thus, a non-naturally occurring nucleic acid is considered to be exogenous to a host once in the host. It is important to note that non-naturally occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally occurring nucleic acid. It follows that genomic DNA fragments produced by PCR or restriction endonuclease treatment as well as cDNAs are considered to be non-naturally occurring nucleic acid since they exist as separate molecules not found in nature. An exogenous sequence may therefore be integrated into the genome of the host. It also follows that any nucleic acid containing a promoter sequence and polypeptide-encoding sequence (e.g., cDNA or genomic DNA) in an arrangement not found in nature is non-naturally occurring nucleic acid. A nucleic acid that is naturally occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

Flagella are filamentous protein structures found in bacteria, archaea, and eukaryotes, though they are most commonly found in bacteria. They are typically used to propel a cell through liquid (i.e., bacteria and sperm). However, flagella have many other specialized functions. Flagella are usually found in gram-negative bacilli. Gram-positive rods (e.g., Listeria species) and cocci (some Enterococcus species, Vagococcus species) also have flagella.

Engineered Salmonella could be any strain of Salmonella designed to lyse and deliver protein intracellularly.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” is an amount sufficient to effect beneficial or desired result, such as a preclinical or clinical result. An effective amount can be administered in one or more administrations. The term “effective amount,” as applied to the compound(s), biologics and pharmaceutical compositions described herein, means the quantity necessary to render the desired therapeutic result. For example, an effective amount is a level effective to treat, cure, or alleviate the symptoms of a disorder and/or disease for which the therapeutic compound, biologic or composition is being administered. Amounts effective for the particular therapeutic goal sought will depend upon a variety of factors including the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage can occur depending upon the condition of the subject being treated, and the physician or other individual administering treatment will, in any event, determine the appropriate dose for an individual patient.

As used herein, “disorder” refers to a disorder, disease or condition, or other departure from healthy or normal biological activity, and the terms can be used interchangeably. The terms would refer to any condition that impairs normal function. The condition may be caused by sporadic or heritable genetic abnormalities. The condition may also be caused by non-genetic abnormalities. The condition may also be caused by injuries to a subject from environmental factors, such as, but not limited to, cutting, crushing, burning, piercing, stretching, shearing, injecting, or otherwise modifying a subject's cell(s), tissue(s), organ(s), system(s), or the like.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, RNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, an “essentially pure” preparation of a particular protein or peptide is a preparation wherein at least about 95%, and including at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double stranded DNA and cDNA. Furthermore, the terms, “nucleic acid,” “DNA,” “RNA” and similar terms also include nucleic acid analogs, i.e., analogs having other than a phosphodiester backbone. For example, the so called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G. C) in which “U” replaces “T.”

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence, e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; preferably in 7% (SDS), 0.5 M NaPO4, 1 mM EDTA at 50° C. with washing in 1×SSC, 0.1% SDS at 50° C.; preferably 7% SDS, 0.5 M NaPO4, 1 mM EDTA at 50° C. with washing in 0.5×SSC, 0.1% SDS at 50° C.; and more preferably in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

### I. Bacteria

Bacteria useful in the invention include, but are not limited to, Clostridium, Bifidus, Escherichia coli or Salmonella, T3SS-dependent bacteria, such as shigella, salmonella and Yersinia Pestis. Further, E. coli can be used if the T3SS system is place in E. Coli.

### Salmonella

Examples of Salmonella strains which can be employed in the present invention include Salmonella typhi (ATCC No. 7251) and S. typhimurium (ATCC No. 13311). Attenuated Salmonella strains include S. typhi-aroC-aroD (Hone et al. Vacc. 9:810 (1991) S. typhimurium-aroA mutant (Mastroeni et al. Micro, Pathol. 13:477 (1992)) and Salmonella typhimurium 7207. Additional attenuated Salmonella strains that can be used in the invention include one or more other attenuating mutations such as (i) auxotrophic mutations, such as aro (Hoiseth et al. Nature, 291:238-239 (1981)), gua (McFarland et al Microbiol. Path., 3:129-141 (1987)), nad (Park et al. J. Bact, 170:3725-3730 (1988), thy (Nnalue et al. Infect. Immun., 55:955-962 (1987)), and asd (Curtiss, supra) mutations; (ii) mutations that inactivate global regulatory functions, such as cya (Curtiss et al. Infect. Immun., 55:3035-3043 (1987)), crp (Curtiss et al (1987), supra), phoP/phoQ (Groisman et al. Proc. Natl. Acad. Sci., USA, 86:7077-7081 (1989); and Miller et al. Proc. Natl. Acad. Sci., USA, 86:5054-5058 (1989)), phop.sup.c (Miller et al. J. Bact, 172:2485-2490 (1990)) or ompR (Dorman et al. Infect. Immun., 57:2136-2140 (1989)) mutations; (iii) mutations that modify the stress response, such as recA (Buchmeier et al. Mol. Micro., 7:933-936 (1993)), htrA (Johnson et al. Mol. Micro., 5:401-407 (1991)), htpR (Neidhardt et al. Biochem. Biophys. Res. Com., 100:894-900 (1981)), hsp (Neidhardt et al. Ann. Rev. Genet, 18:295-329 (1984)) and groEL (Buchmeier et al. Sci., 248:730-732 (1990)) mutations; mutations in specific virulence factors, such as IsyA (Libby et al. Proc. Natl. Acad. Sci., USA, 91:489-493 (1994)), pag or prg (Miller et al (1990), supra; and Miller et al (1989), supra), iscA or virG (d′Hauteville et al. Mol. Micro., 6:833-841 (1992)), plcA (Mengaud et al. Mol. Microbiol., 5:367-72 (1991); Camilli et al. J. Exp. Med, 173:751-754 (1991)), and act (Brundage et al. Proc. Natl. Acad. Sci., USA, 90:11890-11894 (1993)) mutations; (v) mutations that affect DNA topology, such as top A (Galan et al. Infect. Immun., 58:1879-1885 (1990)); (vi) mutations that disrupt or modify the cell cycle, such as min (de Boer et al. Cell, 56:641-649 (1989)); (vii) introduction of a gene encoding a suicide system, such as sacB (Recorbet et al. App. Environ. Micro., 59:1361-1366 (1993); Quandt et al. Gene, 127:15-21 (1993)), nuc (Ahrenholtz et al. App. Environ. Micro., 60:3746-3751 (1994)), hok, gef, kil, or phIA (Molin et al. Ann. Rev. Microbiol., 47:139-166 (1993)); (viii) mutations that alter the biogenesis of lipopolysaccharide and/or lipid A, such as rFb (Raetz in Escherichia coli and Salmonella typhimurium, Neidhardt et al, Ed., ASM Press, Washington D.C. pp 1035-1063 (1996)), galE (Hone et al. J. Infect. Dis., 156:164-167 (1987)) and htrB (Raetz, supra), msbB (Reatz, supra; and U.S. Pat. No. 7,514,089); and (ix) introduction of a bacteriophage lysis system, such as lysogens encoded by P22 (Rennell et al. Virol, 143:280-289 (1985)), lamda murein transglycosylase (Bienkowska-Szewczyk et al. Mol. Gen. Genet., 184:111-114 (1981)) or S-gene (Reader et al. Virol, 43:623-628 (1971)).

The attenuating mutations can be either constitutively expressed or under the control of inducible promoters, such as the temperature sensitive heat shock family of promoters (Neidhardt et al. supra), or the anaerobically induced nirB promoter (Harbome et al. Mol. Micro., 6:2805-2813 (1992)) or repressible promoters, such as uapA (Gorfinkiel et al. J. Biol. Chem., 268:23376-23381 (1993)) or gcv (Stauffer et al. J. Bact, 176:6159-6164 (1994)).

In one embodiment, the bacterial delivery system is safe and based on a non-toxic, attenuated Salmonella strain that has a partial deletion of the msbB gene. This deletion diminishes the TNF immune response to bacterial lipopolysaccharides and prevents septic shock. In another embodiment, it also has a partial deletion of the purl gene. This deletion makes the bacteria dependent on external sources of purines and speeds clearance from non-cancerous tissues (13). In mice, the virulence (LD50) of the therapeutic strain is 10,000-fold less than wild-type Salmonella (72, 73). In pre-clinical trials, attenuated Salmonella has been administered systemically into mice and dogs without toxic side effects (17, 27). Two FDA-approved phase I clinical trials have been performed and showed that this therapeutic strain can be safely administered to patients (20). In one embodiment, the strain of bacteria is VNP20009, a derivative strain of Salmonella typhimurium. Deletion of two of its genes-msbB and purl-resulted in its complete attenuation (by preventing toxic shock in animal hosts) and dependence on external sources of purine for survival. This dependence renders the organism incapable of replicating in normal tissue such as the liver or spleen, but still capable of growing in tumors where purine is available.

Further, insertion of a failsafe circuit into the bacterial vector prevents unwanted infection and defines the end of therapy without the need for antibiotics to remove the bacteria (e.g., salmonella).

### Flagella

### 1) AhDC Sequence

In one aspect, the flhDC sequence is the bicistronic, flhDC coding region found in the Salmonella Typhimurium 14028s strain or a derivative thereof

### Accession Number—

fhD-NCBI Reference Sequence: NC_016856.1
flhC-NCBI Reference Sequence: NC_016856.1

Bicistronic DNA sequence
(SEQ ID NO: 1)
ATGCATACATCCGAGTTGCTAAAACACATTTATGACATCAATTTGTCATA
TTTACTCCTTGCACAGCGTTTGATCGTCCAGGACAAAGCATCTGCGATGT
TCCGCCTCGGTATCAACGAAGAGATGGCAAACACACTGGGCGCGTTGACC
CTGCCGCAGATGGTCAAACTGGCGGAGACGAACCAGTTAGTTTGTCATTT
CCGGTTTGACGATCATCAGACGATCACCCGTTTGACTCAGGATTCGCGCG
TCGATGACTTACAGCAGATTCACACAGGTATCATGCTTTCAACGCGTCTG
CTCAATGAAGTGGACGATACGGCGCGTAAGAAAAGGGCATGATAATGAGT
GAAAAAAGCATTGTTCAGGAAGCTCGCGATATCCAGTTGGCGATGGAGTT
GATTAATCTTGGCGCTCGTCTACAAATGCTGGAAAGCGAAACACAGCTCA
GCCGTGGTCGCCTCATCAGGCTGTACAAAGAATTACGCGGTAGCCCGCCG
CCTAAAGGGATGCTGCCATTTTCGACAGACTGGTTTATGACCTGGGAGCA
AAATATTCATGCCTCCATGTTCTGCAACGCCTGGCAATTTTTACTGAAGA
CCGGCTTATGCAGCGGTGTGGATGCGGTGATTAAAGCTTATCGGCTTTAT
CTTGAGCAGTGTCCGCAACCGCCTGAAGGGCCGTTGTTGGCGCTGACTCG
CGCATGGACGCTGGTGCGTTTTGTTGAAAGTGGGTTGCTTGAATTGTCGA
GCTGTAACTGCTGCGGTGGGAACTTTATTACCCATGCGCATCAGCCCGTA
GGCAGCTTTGCGTGTAGTTTATGCCAGCCGCCATCCCGCGCAGTAAAAAG
ACGTAAACTTTCCCGAGATGCTGCCGATATTATTCCACAACTGCTGGATG
AACAGATCGAACAGGCTGTTTAA
Protein sequence
flhD
(SEQ ID NO: 2)
MHTSELLKHIYDINLSYLLLAQRLIVQDKASAMFRLGINEEMANTIGALI
LPQMVKLAETNQLVCHFRFDDHQTITRLTQDSRVDDLQQIHTGIMLSTRL
LNEVDDTARKKRA
flhC
(SEQ ID NO: 3)
MSEKSIVQEARDIQLAMELINLGARLQMLESETQLSRGRLIRLYKELRGS
PPPKGMLPFSTDWFMTWEQNIHASMFCNAWQFLLKTGLCSGVDAVIKAYR
LYLEQCPQPPEGPLLALTRAWTLVRFVESGLLELSSCNCCGGNFITHAHQ
PVGSFACSLCQPPSRAVKRRKLSRDAADIIPQLLDEQIEQAV

Other sequences can also be used to control flagella activity, these include, for example, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL.

motA, WP_000906312.1
>WP_000906312. 1 MULTISPECIES: flagellar motor stator protein MotA
[Salmonella]
(SEQ ID NO: 4)
MLILLGYLVVIGTVFGGYVMTGGHLGALYQPAELVIIGGAGIGAFIVGNNGKAIKGTMKAIPLLFRRSKYTKSMY
MDLLALLYRLMAKSRQQGMFSLERDIENPKESEIFASYPRILADAVMLDFIVDYLRLIISGNMNTFEIEALMDEE
IETHESEAEVPANSLAMVGDSLPAFGIVAAVMGVVHALASADRPAAELGALIAHAMVGTFLGILLAYGFISPLAT
VLRQKSAETTKMMQCVKITLLSNLNGYAPPIAVEFGRKTLYSSERPSFIELEEHVRAVRNPNQQQTTEEA
motB, WP_000795653.1
>WP_000795653. 1 MULTISPECIES: flagellar motor protein MotB [Salmonella]
(SEQ ID NO: 5)
MKNQAHPIVVVKRRRHKPHGGGAHGSWKIAYADFMTAMMAFFLVMWLISISSPKELIQIAEYFRTPLATAVTGGN
RIANSESPIPGGGDDYTQQQGEVEKQPNIDELKKRMEQSRLNKLRGDLDQLIESDPKLRALRPHLKIDLVQEGLR
IQIIDSQNRPMFKTGSAEVEPYMRDILRAIAPVLNGIPNRISLAGHTDDFPYANGEKGYSNWELSADRANASRRE
LVAGGLDNGKVLRVVGMAATMRLSDRGPDDAINRRISLLVLNKQAEQAILHENAESQNEPVSVLQQPAAAPPASV
PTSPKAEPR
flhE, WP_001233619.1
>WP_001233619.1 MULTISPECIES: flagellar protein FlhE [Salmonella]
(SEQ ID NO: 6)
MRKWLALLLFPLTVQAAGEGAWQDSGMGVTLNYRGVSASSSPLSARQPVSGVMTLVAWRYELNGPTPAGLRVRLC
SQSRCVELDGQSGTTHGFAHVPAVEPLRFVWEVPGGGRLIPALKVRSNQVIVNYR
cheZ, WP_000983586.1
>WP_000983586.1 MULTISPECIES; protein phosphatase Chez [Salmonella]
(SEQ ID NO: 7)
MMQPSIKPADEGSAGDIIARIGSLTRMLRDSLRELGLDQAIAEAAEAIPDARDRLDYVVQMTAQAAERALNSVEA
SQPHQDAMEKEAKALTQRWDEWFDNPIELSDARELVTDTRQFLRDVPGHTSFTNAQLLDIMMAQDFQDLTGQVIK
RMMDVIQEIERQLLMVLLENIPEQSARPKRENESLINGPQVDTSKAGVVASQDQVDDLLDSLGF
cheY WP_000763861.1
>WP_000763861.1 MULTISPECIES: chemotaxis response regulator CheY
[Salmonella]
(SEQ ID NO: 8)
MADKELKFLVVDDFSTMRRIVRNLLKELGFNNVEEAEDGVDALNKLQAGGFGFIISDWNMPNMDGLELLKTIRAD
SAMSALPVLMVTAEAKKENIIAAAQAGASGYVVKPFTAATLEEKLNKIFEKLGM
cheB, WP_000036392.1
>WP_000036392.1 MULTISPECIES; protein-glutamate methylesterase/protein
glutamine deamidase [Salmonella]
(SEQ ID NO: 9)
MSKIRVLSVDDSALMRQIMTEIINSHSDMEMVATAPDPLVARDLIKKFNPDVLILDVEMPRMDGLDFLEKLMRLR
PMPVVMVSSLTGKGSEVTLRALELGAIDFVTKPQLGIREGMLAYSEMIAEKVRTAARARIAAHKPMAAPTTLKAG
PLLSSEKLIAIGASTGGTEAIRHVLQPLPLSSPAVIITQHMPPGFTRSFAERINKLCQISVKEAEDGERVLPGHA
YIAPGDKHMELARSGANYQIKIHDGPPVNRHRPSVDVLFHSVAKHAGRNAVGVILTGMGNDGAAGMLAMYQAGAW
TIAQNEASCVVFGMPREAINMGGVSEVVDLSQVSQOMLAKISAGQAIRI
cheR, WP_000204362.1
>WP_000204362, 1 MULTISPECIES; protein-glutamate O-methyltransferase CheR
[Salmonella]
(SEQ ID NO: 10)
MTSSLPSGQTSVLLQMTQRLALSDAHFRRICQLIYQRAGIVLADHKRDMVYNRLVRRLRALGLDDFGRYLSMLEA
NQNSAEWQAFINALTTNLTAFFREAHHFPILAEHARRRHGEYRVWSAAASTGEEPYSIAITLADALGMAPGRWKV
FASDIDTEVLEKARSGIYRLSELKTLSPQQLQRYFMRGTGPHEGLVRVRQELANYVEFSSVNLLEKQYNVPGPFD
AIFCRNVMIYFDKTTQEDILRRFVPLLKPDGLLFAGHSENFSNLVREFSLRGQTVYALSKDKA
cheM, WP_000483274.1
>WP_000483274.1 MULTISPECIES: methyl-accepting chemotaxis protein II
[Salmonella]
(SEQ ID NO: 11)
MFNRIRVVTMLMMVLGVFALLQLVSGGLLESSLQHNQQGFVISNELRQQQSELTSTWDLMLQTRINLSRSAARMM
MDASNQQSSAKTDLLQNAKTTLAQAAAHYANFKNMTPLPAMAEASANVDEKYQRYQAALAELIQFLDNGNMDAYF
AQPTQGMQNALGEALGNYARVSENLYRQTFDQSAHDYRFAQWQLGVLAVVLVLILMVVWFGIRHALLNPLARVIT
HIREIASGDLTKTLTVSGRNEIGELAGTVEHMQRSLIDTVTQVREGSDAIYSGTSEIAAGNTDLSSRTEQQASAL
EETAASMEQLTATVKQNADNARQASQLAQSASETARHGGKVVDGVVNIMHEIADSSKKIADIISVIDGIAFQTNI
LALNAAVEAARAGEQGRGFAVVAGEVRNLASRSAQAAKEIKALIEDSVSRVDTGSVLVESAGETMTDIVNAVTRV
TDIMGEIASASDEQSRGIDQVALAVSEMDRVTQQNASLVQESAAAAAALEEQASRLTQAVSAFRLASRPLAVNKP
EMRLSVNAQSGNTPQSLAARDDANWETF
cheW, WP_000147295.1
>WP_ 000147295, 1 MULTISPECIES; chemotaxis protein CheW [Salmonella]
(SEQ ID NO: 12)
MTGMSNVSKLAGEPSGQEFLVFTLGNEEYGIDILKVQEIRGYDQVTRIANTPAFIKGVTNLRGVIVPIVDLRVKF
CEGDVEYDDNTVVIVLNLGQRVVGIVVDGVSDVLSLTAEQIRPAPEFAVTLSTEYLTGLGALGERMLILVNIEKL
LNSEEMALLDIAASHVA
cheA, WP_000061302.1
>WP_000061302.1 MULTISPECIES: chemotaxis protein CheA [Salmonella]
(SEQ ID NO: 13)
MSMDISDFYQTFFDEADELLADMEQHLLDLVPESPDAEQLNAIFRAAHSIKGGAGTFGFTILQETTHLMENLLDE
ARRGEMQLNTDIINLFLETKDIMQEQLDAYKNSEEPDAASFEYICNALRQLALEAKGETTPAVVETAALSAAIQE
ESVAETESPRDESKLRIVLSRLKANEVDLLEEELGNLATLIDVVKGADSLSATLDGSVAEDDIVAVLCFVIEADQ
IAFEKVVAAPVEKAQEKTEVAPVAPPAVVAPAAKSAAHEHHAGREKPARERESTSIRVAVEKVDQLINLVGELVI
TQSMLAQRSNELDPVNHGDLITSMGQLQRNARDLQESVMSIRMMPMEYVESRFPRLVRDLAGKLGKQVELTLVGS
STELDKSLIERIIDPLTHLVRNSLDHGIEMPEKRLEAGKNVVGNLILSAEHQGGNICIEVTDDGAGLNRERILAK
AMSQGMAVNENMTDDEVGMLIFAPGFSTAEQVTDVSGRGVGMDVVKRNIQEMGGHVEIQSKQGSGTTIRILLPLT
LAILDGMSVRVAGEVFILPLNAVMESLQPREEDLHPLAGGERVLEVRGEYLPLVELWKVFDVDGAKTEATQGIVV
ILQSAGRRYALLVDQLIGQHQVVVKNLESNYRKVPGISAATILGDGSVALIVDVSALQGLNREQRMAITAA
fliA, WP_001087453.1
>WP_001087453.1 MULTISPECIES: RNA polymerase sigma factor FliA [Salmonella]
(SEQ ID NO: 14)
MNSLYTAEGVMDKHSLWQRYVPLVRHEALRLQVRLPASVELDDLLQAGGIGLLNAVDRYDALQGTAFTTYAVQRI
RGAMLDELRSRDWVPRSVRRNAREVAQAMGQLEQELGRNATETEVAERLGIPVAEYRQMLLDTNNSQLFSYDEWR
EEHGDSIELVTEEHQQENPLHQLLEGDLRQRVMDAIESLPEREQLVLTLYYQEELNLKEIGAVLEVGESRVSQLH
SQAIKRLRTKLGKL
fliY, WP_ 000761635.1
>WP_000761635.1 MULTISPECIES; cystine ABC transporter substrate-binding
protein [Salmonella]
(SEQ ID NO: 15)
MKLALLGRQALMGVMAVALVAGMSAKSFADEGLLNKVKERGTLLVGLEGTYPPFSFOGEDGKLTGFEVDFAEALA
KHLGVKASLKPTKWDGMLASLDAKRIDVVINQVTISDVRKKKYDFSTPYTVSGIQALVKKGNEGTIKTAADLQGK
KVGVGLGTNYEEWLRQHVQGVDIRTYDDDPTKYQDLRVGRIDAILVDRLAALDLVKKTKGTLAVTGDAFSRQESG
VALRKGNEDLLKAVDNAIAEMQKDGTLKALSEKWFGADVTQ
fliZ, WP_000218080.1
>WP_000218080. 1 MULTISPECIES: flagella biosynthesis regulatory protein FliZ
[Salmonella]
(SEQ ID NO: 16)
MTVQQPKRRPLSRYLKDFKHSQTHCAHCHKLLDRITLVRRGKIVNKIAISQLDMLLDDAAWQREQKEWVALCRFC
GDLHCKKQSDFFDIIGFKQYLFEQTEMSHGTVREYVVRLRRIGNYLSEQNISHDLLQDGFLDESLAPWLPETSTN
NYRIALRKYQQYKAHQQIAPRQKSPFTASSDIY
fliB, WP_000079794.1
>WP_000079794.1 MULTISPECIES: FliC/FljB family flagellin [Salmonella]
(SEQ ID NO: 17)
MAQVINTNSLSLLTQNNLNKSQSALGTAIERLSSGLRINSAKDDAAGQAIANRFTANIKGLTQASRNANDGISIA
QTTEGALNEINNNLQRVRELAVQSANSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDVSGLDDAAIKAATGGTNGTASVTGGA
VKFDADNNKYFVTIGGFTGADAAKNGDYEVNVAIDGTVTLAAGATKTIMPAGATTKTEVQELKDTPAVVSADAKN
ALIAGGVDATDANGAELVKMSYTDKNGKTIEGGYALKAGDKYYAADYDEATGAIKAKTTSYTAADGTTKTAANQL
GGVDGKTEVVTIDGKTYNASKAAGHDFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQNVLSLLR
fliS, WP_000287764. 1
>WP_000287764. 1 MULTISPECIES: flagellar export chaperone FliS [Salmonella]
(SEQ ID NO: 18)
MYTASGIKAYAQVSVESAVMSASPHQLIEMLFDGANSALVRARLFLEQGDVVAKGEALSKAINIIDNGLKAGLDQ
EKGGEIATNLSELYDYMIRRLLQANLRNDAQAIEEVEGLLSNIAEAWKQISPKASFQESR
fliE, WP_000719036.1
>WP_000719036.1 MULTISPECIES: flagellar hook-basal body complex protein
FliE [Salmonella]
(SEQ ID NO: 19)
MAAIQGIEGVISQLQATAMAARGQDTHSQSTVSFAGQLHAALDRISDRQAAARVQAEKFTLGEPGIALNDVMADM
QKASVSMQMGIQVRNKLVAAYQEVMSMQV
fliF, WP_ 001276834.1
>WP_001276834.1 MULTISPECIES: flagellar M-ring protein FliF [Salmonella]
(SEQ ID NO: 20)
MSATASTATQPKPLEWINRLRANPRIPLIVAGSAAVAIVVAMVLWAKTPDYRTLFSNLSDQDGGAIVAQLTQMNI
PYRFANGSGAIEVPADKVHELRERLAQQGLPKGGAVGFELLDQEKFGISQFSEQVNYQRALEGELARTIETLGPV
KSARVHLAMPKPSLFVREQKSPSASVTVTLEPGRALDEGQISAVVHLVSSAVAGLPPGNVTLVDQSGHLLTQSNT
SGRDLNDAQLKFANDVESRIQRRIEAILSPIVGNGNVHAQVTAQLDFANKEQTEEHYSPNGDASKATLRSRQLNI
SEQVGAGYPGGVPGALSNQPAPPNEAPIATPPTNQQNAQNTPQTSTSTNSNSAGPRSTQRNETSNYEVDRTIRHT
KMNVGDIERLSVAVVVNYKTLADGKPLPLTADQMKQIEDLTREAMGESDKRGDTLNVVNSPFSAVDNTGGELPFW
QQQSFIDQLLAAGRWLLVLVVAWILWRKAVRPQLTRRVEEAKAAQEQAQVRQETEEAVEVRLSKDEQLQQRRANQ
RLGAEVMSQRIREMSDNDPRVVALVIRQWMSNDHE
fliJ, WP_000046981.1
>WP_000046981.1 MULTISPECIES: flagella biosynthesis chaperone FliJ
[Salmonella]
(SEQ ID NO: 21)
MAQHGALETLKDLAEKEVDDAARLLGEMRRGCQQAEEQLKMLIDYQNEYRSNLNTDMGNGIASNRWINYQQFIQT
LEKAIEQHRLQLTQWTQKVDLALKSWREKKQRLQAWQTLQDRQTAAALLAENRMDQKKMDEFAQRAAMRKPE
fliL, WP_000132169.1
>WP_000132169. 1 MULTISPECIES: flagellar basal body-associated protein FliL
[Salmonella]
(SEQ ID NO: 22)
MTDSAINKKSKRSIWIPLLVLITLAACATAGYSYWRMQQQPTTNAKAEPAPPPAPVFFALDTFTVNLGDADRVLY
IGVTLRLKDEATRARLNEYLPEVRSRLLLLFSRQNAAELSTEAGKQKLIAAIKETLAAPLVAGQPKQVVTDVLYT
AFILR
fliM, WP_000502811.1
>WP_000502811.1 MULTISPECIES: flagellar motor switch protein FliM
[Salmonella]
(SEQ ID NO: 23)
MGDSILSQAEIDALLNGDSDTKDEPTPGIASDSDIRPYDPNTQRRVVRERLQALEIINERFARQFRMGLFNLLRR
SPDITVGAIRIQPYHEFARNLPVPTNLNLIHLKPLRGTGLVVFSPSLVFIAVDNLFGGDGRFPTKVEGREFTHTE
QRVINRMLKLALEGYSDAWKAINPLEVEYVRSEMQVKFTNITTSPNDIVVNTPFHVEIGNLTGEFNICLPFSMIE
PLRELLVNPPLENSRHEDQNWRDNLVRQVQHSELELVANFADIPLRLSQILKLKPGDVLPIEKPDRIIAHVDGVP
VLTSQYGTVNGQYALRVEHLINPILNSLNEEQPK
fliN, WP_001282115.1
>WP_001282115.1 MULTISPECIES: flagellar motor switch protein FliN
[Salmonella]
(SEQ ID NO: 24)
MSDMNNPSDENTGALDDLWADALNEQKATTTKSAADAVFQQLGGGDVSGAMQDIDLIMDIPVKLTVELGRTRMTI
KELLRLTQGSVVALDGLAGEPLDILINGYLIAQGEVVVVADKYGVRITDIITPSERMRRLSR
fliO, WP_000978276.1
>WP_000978276.1 MULTISPECIES: flagellar type III secretion system protein
FliO [Salmonella]
(SEQ ID NO: 25)
MMKTEATVSQPTAPAGSPLMQVSGALIGIIALILAAAWVIKRMGFAPKGNSVRGLKVSASASLGPRERVVIVEVE
NARLVLGVTASQINLLHTLPPAENDTEAPVAPPADFQNMMKSLLKRSGRS
fliP, WP_001253410.1
>WP_001253410.1 MULTISPECIES: flagellar type III secretion system pore
protein FliP [Salmonella]
(SEQ ID NO: 26)
MRRLLFLSLAGLWLFSPAAAAOLPGLISQPLAGGGQSWSLSVQTLVFITSLTFLPAILLMMTSFTRIIIVFGLLR
NALGTPSAPPNQVLLGLALFLTFFIMSPVIDKIYVDAYQPFSEQKISMQEALDKGAQPLRAFMLRQTREADLALF
ARLANSGPLQGPEAVPMRILLPAYVTSELKTAFQIGFTIFIPFLIIDEVIASVLMALGMMMVPPATIALPFKLML
FVLVDGWQLLMGSLAQSFYS
fliQ, WP. 000187355.1
>WP_000187355.1 MULTISPECIES: flagellar biosynthesis protein FliQ
[Salmonella]
(SEQ ID NO: 27)
MTPESVMMMGTEAMKVALALAAPLLLVALITGLIISILQAATQINEMTLSFIPKIVAVFIAIIVAGPWMLNLLLD
YVRTLFSNLPYIIG
fliR, WP_000616953.1
>WP_ 000616953, 1 MULTISPECIES; flagellar type III secretion system protein
FliR [Salmonella]
(SEQ ID NO: 28)
MIQVTSEQWLYWLHLYFWPLLRVLALISTAPILSERAIPKRVKLGLGIMITLVIAPSLPANDTPLFSIAALWLAM
QQILIGIALGFTMQFAFAAVRTAGEFIGLQMGLSFATFVDPGSHLNMPVLARIMDMLAMLLFLTFNGHLWLISLL
VDTFHTLPIGSNPVNSNAFMALARAGGLIFLNGLMLALPVITLLLTLNLALGLLNRMAPQLSIFVIGFPLTLTVG
IMLMAALMPLIAPFCEHLFSEIFNLLADIVSEMPINNNP
fliG, WP_000067735.1
>WP_000067735.1 MULTISPECIES: flagellar motor switch protein FliG
[Salmonella]
(SEQ ID NO: 29)
MSNLSGTDKSVILLMTIGEDRAAEVFKHLSTREVQALSTAMANVRQISNKQLTDVLSEFEQEAEQFAALNINANE
YLRSVLVKALGEERASSLLEDILETRDTTSGIETLNFMEPQSAADLIRDEHPQIIATILVHLKRSQAADILALFD
ERLRHDVMLRIATEGGVQPAALAELTEVLNGLLDGQNLKRSKMGGVRTAAEIINLMKTQQEEAVITAVREFDGEL
AQKIIDEMFLFENLVDVDDRSIQRLLQEVDSESLLIALKGAEPPLREKFLRNMSQRAADILRDDLANRGPVRLSQ
VENEQKAILLIVRRLAETGEMVIGSGEDTYV
fliH, WP_000064163.1
>WP_000064163. 1 MULTISPECIES: flagellar assembly protein FliH [Salmonella]
(SEQ ID NO: 30)
MSNELPWQVWTPDDLAPPPETFVPVEADNVTLTEDTPEPELTAEQQLEQELAQLKIQAHEQGYNAGLAEGRQKGH
AQGYQEGLAQGLEQGQAQAQTQQAPIHARMQQLVSEFQNTLDALDSVIASRLMQMALEAARQVIGQTPAVDNSAL
IKQIQQLLQQEPLFSGKPQLRVHPDDLQRVEEMLGATLSLHGWRLRGDPTLHHGGCKVSADEGDLDASVATRWQE
LCRLAAPGVL
fliI, WP_000213257. 1
>WP_000213257.1 MULTISPECIES: flagellum-specific ATP synthase FliI
[Salmonella]
(SEQ ID NO: 31)
MTTRLTRWLTALDNFEAKMALLPAVRRYGRLTRATGLVLEATGLQLPLGATCIIERQDGPETKEVESEVVGFNGQ
RLFLMPLEEVEGILPGARVYARNGHGDGLQSGKQLPLGPALLGRVLDGGGKPLDGLPAPDTLETGALITPPFNPL
QRTPIEHVLDTGVRAINALLTVGRGQRMGLFAGSGVGKSVLLGMMARYTRADVIVVGLIGERGREVKDFIENILG
PDGRARSVVIAAPADVSPLLRMQGAAYATRIAEDFRDRGQHVLLIMDSLTRYAMAQREIALAIGEPPATKGYPPS
VFAKLPALVERAGNGIHGGGSITAFYTVLTEGDDQQDPIADSARAILDGHIVLSRRLAEAGHYPAIDIEASISRA
MTALITEQHYARVRLFKQLLSSFQRNRDLVSVGAYAKGSDPMLDKAITLWPQLEAFLQQGIFERADWEDSLQALD
LIFPTV
fliT, WP_000204899.1
>WP_000204899. 1 MULTISPECIES: flagella biosynthesis regulatory protein FliT
[Salmonella]
(SEQ ID NO: 32)
MISTVEFINRWQRIALLSQSLLELAQRGEWDLLLQQEVSYLQSIETVMEKQTPPGITRSIQDMVAGYIKQTLDNE
QLLKGLLQQORLDELSSLIGQSTRQKSLNNAYGRLSGMLLVPDAPGAS
fliD, WP_000146802.1
>WP_ 000146802.1 MULTISPECIES: flagellar filament capping protein FliD
[Salmonella]
(SEQ ID NO: 33)
MASISSIGVGSNLPLDQLLTDLTKNEKGRLTPITKQQSANSAKLTAYGTLKSALEKFQTANTALNKADLFKSTVA
SSTTEDLKVSTTAGAAAGTYKINVTQLAAAQSLATKITFATTKEQLGDISVTSRTIKIEQPGRKEPLEIKLDKGD
TSMEAIRDAINDADSGIAASIVKVKENEFQLVLTANSGTDNTMKITVEGDTKLNDLLAYDSTTNTGNMQELVKAE
NAKLNVNGIDIERQSNTVIDAPQGITLTLTKKVTDATVTVTKDDTKAKEAIKSWVDAYNSLVDTFSSLTKYTAVE
PGEEASDKNGALLGDSVVRTIQTGIRAQFANSGSNSAFKTMAEIGITQDGTSGKLKIDDDKLIKVLKDNTAAARE
LLVGDGKETGITTKIATEVKSYLADDGIIDNAQDNVNATLKSLIKQYLSVSNSIDETVARYKAQFTQLDTMMSKL
NNTSSYLTQOFTAMNKS
fliC, WP_ 000079805.1
>WP_000079805.1 MULTISPECIES: FliC/FljB family flagellin [Salmonella]
(SEQ ID NO: 34)
MAQVINTNSLSLLTQNNLNKSQSALGTAIERLSSGLRINSAKDDAAGQAIANRFTANIKGLTQASRNANDGISIA
QTTEGALNEINNNLQRVRELAVQSANSTNSQSDLDSIQAEITQRLNEIDRVSGQTQFNGVKVLAQDNTLTIQVGA
NDGETIDIDLKQINSQTLGLDTLNVQQKYKVSDTAATVTGYADTTIALDNSTFKASATGLGGTDQKIDGDLKFDD
TTGKYYAKVTVTGGTGKDGYYEVSVDKTNGEVTLAGGATSPLTGGLPATATEDVKNVQVANADLTEAKAALTAAG
VTGTASVVKMSYTDNNGKTIDGGLAVKVGDDYYSATQNKDGSISINTTKYTADDGTSKTALNKLGGADGKTEVVS
IGGKTYAASKAEGHNFKAQPDLAEAAATTTENPLQKIDAALAQVDTLRSDLGAVQNRFNSAITNLGNTVNNLTSA
RSRIEDSDYATEVSNMSRAQILQQAGTSVLAQANQVPQNVLSLLR
fljB, WP_000079794.1
>WP_000079794. 1 MULTISPECIES: FliC/FljB family flagellin [Salmonella]
(SEQ ID NO: 35)
MAQVINTNSLSLLTQNNLNKSQSALGTAIERLSSGLRINSAKDDAAGQAIANRFTANIKGLTQASRNANDGISIA
QTTEGALNEINNNLQRVRELAVQSANSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDVSGLDDAAIKAATGGTNGTASVTGGA
VKFDADNNKYFVTIGGFTGADAAKNGDYEVNVATDGTVTLAAGATKTTMPAGATTKTEVQELKDTPAVVSADAKN
ALIAGGVDATDANGAELVKMSYIDKNGKTIEGGYALKAGDKYYAADYDEATGAIKAKITSYTAADGTTKTAANQL
GGVDGKTEVVTIDGKTYNASKAAGHDFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQNVLSLLR
flgN, WP_000197547.1
>WP_000197547.1 MULTISPECIES: flagella biosynthesis chaperone FlgN
[Salmonella]
(SEQ ID NO: 36)
MTRLSEILDQMTTVLNDLKTVMDAEQQQLSVGQINGSQLQRITEEKSSLLATLDYLEQQRRLEQNAQRSANDDIA
ERWQAITEKTQHLRDLNQHNGWLLEGQIERNQQALEVLKPHQEPTLYGADGQTSVSHRGGKKISI
flgM, WP_000020893.1
>WP_000020893.1 MULTISPECIES: anti-sigma-28 factor FlgM [Salmonella]
(SEQ ID NO: 37)
MSIDRTSPLKPVSTVQTRETSDTPVQKTRQEKTSAATSASVTLSDAQAKLMQPGVSDINMERVEALKTAIRNGEL
KMDTGKIADSLIREAQSYLQSK
flgA, WP_001194082.1
>WP_001194082. 1 MULTISPECIES: flagellar basal body P-ring formation protein
FlgA [Salmonella]
(SEQ ID NO: 38)
MQTLKRGFAVAALLFSPLTMAQDINAQLTTWFSQRLAGFSDEVVVTLRSSPNLLPSCEQPAFSMTGSAKLWGNVN
VVARCANEKRYLQVNVQATGNYVAVAAPIARGGKLTPANVTLKRGRLDQLPPRTVLDIRQIQDAVSLRDLAPGQP
VQLTMIRQAWRVKAGQRVQVIANGEGFSVNAEGQAMNNAAVAQNARVRMTSGQIVSGTVDSDGNILINL
flgB, WP_000887043.1
>WP_000887043.1 MULTISPECIES: flagellar basal body rod protein FlgB
[Salmonella]
(SEQ ID NO: 39)
MLDRLDAALRFQQEALNLRAQRQEILAANIANADTPGYQARDIDFASELKKVMVRGREETGGVALTLTSSAHIPA
QAVSSPAVDLLYRVPDQPSLDGNTVDMDRERTQFADNSLKYQMGLTVLGSQLKGMMNVLQGGN
flgC, WP_001196448.1
>WP_001196448. 1 MULTISPECIES: flagellar basal body rod protein FlgC
[Salmonella]
(SEQ ID NO: 40)
MALLNIFDIAGSALAAQSKRLNVAASNLANADSVTGPDGQPYRAKQVVFQVDAAPGQATGGVKVASVIESQAPEK
LVYEPGNPLADANGYVKMPNVDVVGEMVNTMSASRSYQANIEVLNTVKSMMLKTLTLGQ
flgD, WP_000020450.1
>WP_000020450. 1 MULTISPECIES: flagellar hook assembly protein FlgD
[Salmonella]
(SEQ ID NO: 41)
MSIAVNMNDPTNTGVKTTTGSGSMTGSNAADLQSSFLTLLVAQLKNQDPTNPLQNNELTTQLAQISTVSGIEKLN
TTLGAISGQIDNSQSLQATTLIGHGVMVPGTTILAGKGAEEGAVTSTTPFGVELQQPADKVTATITDKDGRVVRT
LEIGELRAGVHTFTWDGKQTDGTTVPNGSYNIAITASNGGTQLVAQPLQFALVQGVTKGSNGNLLDLGTYGTTTL
DEVRQII
flgE, WP_000010567.1
>WP_000010567.1 MULTISPECIES: flagellar hook protein FlgE [Salmonella]
(SEQ ID NO: 42)
MSFSQAVSGLNAAATNLDVIGNNIANSATYGFKSGTASFADMFAGSKVGLGVKVAGITQDFTDGTTTNTGRGLDV
AISQNGFFRLVDSNGSVFYSRNGQFKLDENRNLVNMQGMQLTGYPATGTPPTIQQGANPAPITIPNTLMAAKSTT
TASMQINLNSTDPVPSKTPFSVSDADSYNKKGTVTVYDSQGNAHDMNVYFVKTKDNEWAVYTHDSSDPAATAPTT
ASTTLKFNENGILESGGTVNITTGTINGATAATFSLSFLNSMQQNTGANNIVATNQNGYKPGDLVSYQINNDGTV
VGNYSNEQEQVLGQIVLANFANNEGLASQGDNVWAATQASGVALLGTAGSGNFGKLTNGALEASNVDLSKELVNM
IVAQRNYQSNAQTIKTQDQILNTLVNLR
flgF, WP_000349278.1
>WP_000349278, 1 MULTISPECIES; flagellar basal body rod protein FlgF
[Salmonella]
(SEQ ID NO: 43)
MDHAIYTAMGAASQTLNQQAVTASNLANASTPGFRAQLNALRAVPVDGLSLATRTLVTASTPGADMTPGQLDYTS
RPLDVALQQDGWLVVQAADGAEGYTRNGNIQVGPTGQLTIQGHPVIGEGGPITVPEGSEITIAADGTISALNPGD
PPNTVAPVGRLKLVKAEGNEVQRSDDGLFRITAEAQAERGAVLAADPSIRIMSGVLEGSNVKPVEAMTDMIANAR
RFEMQMKVITSVDENEGRANQLLSMS
flgG, WP_000625851.1
>WP_000625851. 1 MULTISPECIES; flagellar basal-body rod protein FlgG
[Salmonella]
(SEQ ID NO: 44)
MISSLWIAKTGLDAQQTNMDVIANNLANVSTNGFKRQRAVFEDLLYQTIRQPGAQSSEQTTLPSGLQIGTGVRPV
ATERLHSQGNLSQTNNSKDVAIKGQGFFQVMLPDGTSAYTRDGSFQVDQNGQLVTAGGFQVQPAITIPANALSIT
IGRDGVVSVTQQGQAAPVQVGQLNLTTFMNDTGLESIGENLYIETQSSGAPNESTPGLNGAGLLYQGYVETSNVN
VAEELVNMIQVQRAYEINSKAVSTTDQMLQKETQL
flgH, WP_001174897.1
>WP_001174897.1 MULTISPECIES: flagellar basal body L-ring protein FlgH
[Salmonella]
(SEQ ID NO: 45)
MQKYALHAYPVMALMVATLTGCAWIPAKPLVQGATTAQPIPGPVPVANGSIFQSAQPINYGYQPLFEDRRPRNIG
DTLTIVLQENVSASKSSSANASRDGKTSFGFDTVPRYLQGLFGNSRADMEASGGNSFNGKGGANASNTFSGTLTV
TVDQVLANGNLHVVGEKQIAINQGTEFIRFSGVVNPRTISGSNSVPSTQVADARIEYVGNGYINEAQNMGWLQRF
FLNLSPM
flgI, WP_001518955.1
>WP_001518955.1 MULTISPECIES: flagellar basal body P-ring protein FlgI
[Salmonella]
(SEQ ID NO: 46)
MFKALAGIVLALVATLAHAERIRDLTSVQGVRENSLIGYGLVVGLDGTGDQTTQTPFTTQTLNNMLSQLGITVPT
GTNMQLKNVAAVMVTASYPPFARQGQTIDVVVSSMGNAKSLRGGTLLMTPLKGVDSQVYALAQGNILVGGAGASA
GGSSVQVNQLNGGRITNGAIIERELPTQFGAGNTINLQLNDEDETMAQQITDAINRARGYGSATALDARTVQVRV
PSGNSSQVRFLADIQNMEVNVTPQDAKVVINSRTGSVVMNREVTLDSCAVAQGNLSVTVNRQLNVNQPNTPFGGG
QTVVTPQTQIDLRQSGGSLQSVRSSANLNSVVRALNALGATPMDLMSILQSMQSAGCLRAKLEII
flgJ, WP_000578692.1
>WP_000578692.1 MULTISPECIES: flagellar assembly peptidoglycan hydrolase
FlgJ [Salmonella]
(SEQ ID NO: 47)
MIGDGKLLASAAWDAQSLNELKAKAGQDPAANIRPVARQVEGMFVQMMLKSMREALPKDGLFSSDQTRLYTSMYD
QQIAQQMTAGKGLGLADMMVKQMTSGQTMPADDAPQVPLKFSLETVNSYQNQALTQLVRKAIPKTPDSSDAPLSG
DSKDFLARLSLPARLASEQSGVPHHLILAQAALESGWGQRQILRENGEPSYNVFGVKATASWKGPVTEITTTEYE
NGEAKKVKAKFRVYSSYLEALSDYVALLTRNPRYAAVTTAATAEQGAVALQNAGYATDPNYARKLISMIQQLKAM
SEKVSKTYSANLDNLF
flgK, WP_000096425.1
>WP_000096425. 1 MULTISPECIES: flagellar hook-associated protein FlgK
[Salmonella]
(SEQ ID NO: 48)
MSSLINHAMSGLNAAQAALNTVSNNINNYNVAGYTRQTTILAQANSTLGAGGWIGNGVYVSGVQREYDAFITNQL
RGAQNQSSGLTTRYEQMSKIDNLLADKSSSLSGSLQSFFTSLQTLVSNAEDPAARQALIGKAEGLVNQFKTTDQY
LRDQDKQVNIAIGSSVAQINNYAKQIANLNDQISRMTGVGAGASPNDLLDQRDQLVSELNKIVGVEVSVQDGGTY
NLTMANGYTLVQGSTARQLAAVPSSADPTRTTVAYVDEAAGNIEIPEKLLNTGSLGGLLTFRSQDLDQTRNTLGQ
LALAFADAFNAQHTKGYDADGNKGKDFFSIGSPVVYSNSNNADKTVSLTAKVVDSTKVQATDYKIVEDGTDWQVT
RTADNTTFTATKDADGKLEIDGLKVTVGTGAQKNDSFLLKPVSNAIVDMNVKVTNEAEIAMASESKLDPDVDTGD
SDNRNGQALLDLQNSNVVGGNKTFNDAYATLVSDVGNKTSTLKTSSTTQANVVKQLYKQQQSVSGVNLDEEYGNL
QRYQQYYLANAQVLQTANALFDALLNIR
flgL WP_001223033.1
>WP_001223033. 1 MULTISPECIES: flagellar hook-associated protein FlgL
[Salmonella]
(SEQ ID NO: 49)
MRISTQMMYEQNMSGITNSQAEWMKLGEQMSTGKRVTNPSDDPIAASQAVVLSQAQAQNSQYALARTFATQKVSL
EESVLSQVTTAIQTAQEKIVYAGNGTLSDDDRASLATDLQGIRDQLMNLANSTDGNGRYIFAGYKTEAAPFDQAT
GGYHGGEKSVTQQVDSARTMVIGHTGAQIFNSITSNAVPEPDGSDSEKNLFVMLDTAIAALKTPVEGNNVEKEKA
AAAIDKTNRGLKNSLNNVLTVRAELGTQLSELSTLDSLGSDRALGQKLQMSNLVDVDWNSVISSYVMQQAALQAS
YKTFTDMQGMSLFQLNR

### II. Vectors/Plasmids

In the present compositions and/or methods, DNA, RNA (e.g., a nucleic acid-based gene interfering agent) or protein may be produced by recombinant methods. The nucleic acid is inserted into a replicable vector for expression. Many such vectors are available. The vector components generally include, but are not limited to, one or more of the following: an origin of replication, one or more marker genes, an enhancer element, a promoter, and a transcription termination sequence and coding sequence. In some embodiments, for example in the utilization of bacterial delivery agents such as Salmonella, the gene and/or promoter (a sequence of interest) may be integrated into the host cell chromosome or may be presented on, for example, a plasmid/vector.

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors can contain a promoter that is recognized by the host organism and is operably linked to the nucleic acid sequence, such as a nucleic acid sequence coding for an open reading frame. Promoters are untranslated sequences located upstream (5′) to the start codon of a structural gene (generally within about 100 to 1000 bp) that control the transcription of particular nucleic acid sequence to which they are operably linked. In bacterial cells, the region controlling overall regulation can be referred to as the operator. Promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Promoters suitable for use with prokaryotic hosts include the β-lactamase and lactose promoter systems, alkaline phosphatase, a tryptophan (trp) promoter system, hybrid promoters such as the tac promoter, and starvation promoters (Matin, A. (1994) Recombinant DNA Technology II, Annals of New York Academy of Sciences, 722:277-291). However, other known bacterial promoters are also suitable. Such nucleotide sequences have been published, thereby enabling a skilled worker to operably ligate them to a DNA coding sequence. Promoters for use in bacterial systems also can contain a Shine-Dalgarno (S.D.) sequence operably linked to the coding sequence.

Construction of suitable vectors containing one or more of the above-listed components employs standard ligation techniques. Isolated plasmids or DNA fragments are cleaved, tailored, and re-ligated in the form desired to generate the plasmids required.

In some embodiments of the invention, the expression vector is a plasmid or bacteriophage vector suitable for use in Salmonella, and the DNA, RNA and/or protein is provided to a subject through expression by an engineered Salmonella (in one aspect attenuated) administered to the patient. The term “plasmid” as used herein refers to any nucleic acid encoding an expressible gene and includes linear or circular nucleic acids and double or single stranded nucleic acids. The nucleic acid can be DNA or RNA and may comprise modified nucleotides or ribonucleotides and may be chemically modified by such means as methylation or the inclusion of protecting groups or cap- or tail structures.

One embodiment provides a Salmonella strain comprising a lysis gene or cassette operably linked to an intracellularly induced Salmonella promoter. In one embodiment, the promoter is a promoter for one of the genes in Salmonella pathogenicity island 2 type III secretion system (SP12-T3SS) selected from the group SpiC/SsaB (accession no. CBW17423.1), SseF (accession no. CBW17434.1), SseG (accession no. CBW17435.1), SseI (accession no. CBW17087.1), SseJ (accession no. CBW17656.1 or NC_016856.1), SseK1 (accession no. CBW20184.1), SseK2 (accession no. CBW18209.1), SifA (accession no. CBW17257.1), SifB (accession no. CBW17627.1), PipB (accession no. CBW17123.1), PipB2 (accession no. CBW18862.1), SopD2 (accession no. CBW17005.1), GogB (accession no. CBW18646.2), SseL (accession no. CBW18358.1), SteC (accession no. CBW17723.1), SspH/(accession no. STM14_1483), SspH2 (accession no. CBW18313.1), or SirP (examples/an embodiment of sequences that can be used in the instant compositions/methods are provided for by accession numbers and sequences provided throughout the specification; other sequences, including those with greater than about 80%, 85%, 90%, 95%, 96%, 97%, 98%, 99% and 100% identity may also be used in the composition/methods of the invention).

| SpiC/SsaB (accession no. CBW17423.1): | |
|---|---|
| (SEQ ID NO: 50) | |
| 1 | mseegfmlav lkgipliqdi raegnsrswi mtidghparg eifseafsis lflndleslp |
| 61 | kpclayvtll laahpdvhdy aiqltadggw lngyyttsss seliaieiek hlaltcilkn |
| 121 | virnhhklys ggv |
| SseF (accession no. CBW17434.1): | |
| (SEQ ID NO: 51) | |
| 1 | mkihipsaas nivdanspps diqakevsip ppeipapgtp aapvlltpeq irqqrdyaih |
| 61 | fmqytiralg atvvfglsva aavisggagl piailagaal viaigdacca yhnyqsicqq |
| 121 | keplqtasds valvvsalal kcgaslncan tlanclslli rsgiaismlv lplqfplpaa |
| 181 | eniaasldmg svitsvslta igavldycla rpsgddqens vdelhadpsv llaeqmaalc |
| 241 | qsattpalmd ssdhtsrgep |
| SseG (accession no. CBW17435.1): | |
| (SEQ ID NO: 52) | |
| 1 | mkpvspnaqv ggqrpvnape esppcpslph petnmesgri gpqqgkervl aglakrviec |
| 61 | fpkeifswqt vilggqilcc sagialtvls gggaplvala giglaiaiad vacliyhhkh |
| 121 | hlpmahdsig navfyiancf anqrksmaia kavslggrla ltatvmthsy wsgslglqph |
| 181 | llerlndity glmsftrfgm dgmamtgmqv ssplyrllaq vtpeqrape |
| SseI (accession no. CBW17087.1): | |
| (SEQ ID NO: 53) | |
| 1 | mpfhigsgcl paiisnrriy riawsdtppe msswekmkef fcsthqaeal eciwtichpp |
| 61 | agttredvvs rfellrtlay dgweenihsg lhgenyfcil dedsqeilsv tlddvgnytv |
| 121 | ncqgysethh ltmatepgve rtditynlts didaaaylee lkqnpiinnk imnpvgqces |
| 181 | lmtpvsnfmn ekgfdniryr gifiwdkpte eiptnhfavv gnkegkdyvf dvsahqfenr |
| 241 | gmsnlngpli lsadewvcky rmatrrkliy ytdfsnssia anaydalpre lesesmagkv |
| 301 | fvtsprwfnt fkkqkyslig km |
| SseJ (accession no. CBW17656.1): | |
| (SEQ ID NO: 54) | |
| 1 | mplsvgqgyf tssissekfn aikesarlpe lslwekikay fftthhaeal ecifnlyhhq |
| 61 | elnltpvqvr gayiklrala sqgckeqfii esqehadkli ikddngenil sievechpea |
| 121 | fglakeinks hpkpknislg ditrlvffgd slsdslgrmf ekthhilpsy gqyfggrftn |
| 181 | gftwteflss phflgkemln faeggstsas yscfncigdf vsntdrqvas ytpshqdlai |
| 241 | fllgandymt lhkdnvimvv eqqiddieki isggvnnvlv mgipdlsltp ygkhsdekrk |
| 301 | lkdeslahna llktnveelk ekypqhkicy yetadafkvi meaasnigyd tenpythhgy |
| 361 | vhvpgakdpq ldicpqyvfn dlvhptqevh hcfaimlesf iahhyste |
| sseJ sequence (DNA) - Accession number-NCBI Reference Sequence: NC_016856.1 | |
| (SEQ ID NO: 55) | |
| ATGCCATTGAGTGTTGGACAGGGTTATTTCACATCATCTATCAGTTCTGAAAAATTTAATGCGATAAAAGAAAGC | |
| GCACGCCTTCCGGAATTAAGTTTATGGGAGAAAATCAAAGCATATTTCTTTACCACCCACCATGCAGAGGCGCTC | |
| GAATGTATCTTTAATCTTTACCACCATCAGGAACTGAATCTAACACCGGTACAGGTTCGCGGAGCCTACATCAAA | |
| CTTCGAGCCTTAGCGTCTCAGGGATGTAAAGAACAGTTTATTATAGAATCACAGGAACACGCCGATAAGTIGATT | |
| ATTAAAGATGATAATGGTGAAAATATTTTGTCTATTGAGGTTGAATGTCATCCGGAAGCTTTTGGTCTTGCAAAA | |
| GAAATCAATAAATCACATCCCAAGCCCAAAAATATTTCTTTGGGTGATATTACCAGACTGGTATTTTTTGGCGAC | |
| AGCTTGTCTGACTCCTTAGGGCGTATGTTTGAAAAAACACATCATATCTTACCCTCCTATGGTCAATACTTTGGC | |
| GGAAGGTTTACTAATGGATTTACCTGGACTGAGTTTTTATCATCTCCACACTTCTTAGGTAAAGAGATGCTTAAT | |
| TTTGCTGAAGGGGGAAGTACATCGGCAAGCTATTCCTGCTTTAATTGCATCGGTGACTTTGTATCAAATACGGAC | |
| AGACAAGTCGCATCTTACACCCCTTCTCACCAGGACCTGGCGATATTTTTATTGGGGGCTAATGACTATATGACA | |
| CTACACAAAGATAATGTAATAATGGTCGTTGAGCAACAAATTGATGATATTGAAAAAATAATTTCCGGTGGAGTT | |
| AATAATGTTCTGGTCATGGGGATTCCCGATTIGTCTTTAACACCTTATGGCAAACATTCTGATGAAAAAAGAAAG | |
| CTTAAGGATGAAAGCATCGCTCACAATGCCCTGTTAAAAACTAATGTTGAAGAATTAAAAGAAAAATACCCCCAG | |
| CATAAAATATGCTATTACGAGACTGCCGATGCAITTAAGGTGATAATGGAGGCGGCCAGTAATATTGGTTATGAT | |
| ACGGAAAACCCTTATACTCACCACGGCTATGTACATGTTCCCGGGGCTAAAGACCCTCAGCTAGATATATGTCCG | |
| CAATACGTCTTCAACGACCTTGTCCATCCAACCCAGGAAGTCCATCATTGTTTTGCCATAATGTTAGAAAGTTTT | |
| ATAGCTCATCATTATTCCACTGAATAA | |
| sseJ sequence (protein) | |
| (SEQ ID NO: 56) | |
| MPLSVGQGYFTSSISSEKFNAIKESARLPELSLWEKIKAYFFTTHHAEALECIFNLYHHQELNLTPVQVRGAYIK | |
| LRALASQGCKEQFIIESQEHADKLIIKDDNGENILSIEVECHPEAFGLAKEINKSHPKPKNISLGDITRLVFFGD | |
| SLSDSLGRMFEKTHHILPSYGQYFGGRFTNGFTWTEFLSSPHFLGKEMLNFAEGGSTSASYSCFNCIGDFVSNTD | |
| RQVASYTPSHQDLAIFLLGANDYMTLAKDNVIMVVEQQIDDIEKIISGGVNNVLVMGIPDLSLTPYGKHSDEKRK | |
| LKDESIAHNALLKTNVEELKEKYPQHKICYYETADAFKVIMEAASNIGYDTENPYTHHGYVHVPGAKDPQLDICP | |
| QYVFNDLVHPTQEVHACFAIMLESFIAHHYSTE | |
| SseK1 (accession no. CBW20184.1): | |
| (SEQ ID NO: 57) | |
| 1 | mipplnryvp alsknelvkt vtnrdiqfts fngkdyplcf ldektpllfq wfernparfg |
| 61 | kndipiinte knpylnniik aatiekerli gifvdgdffp gqkdafskle ydyenikviy |
| 121 | rndidfsmyd kklseiymen iskqesmpee krdchllqll kkelsdiqeg ndsliksyll |
| 181 | dkghgwfdfy rnmamlkagq lfleadkvgc ydlstnsgci yldadmiite klggiyipdg |
| 241 | iavhveridg rasmengiia vdrnnhpall agleimhtkf dadpysdgvc ngirkhfnys |
| 301 | lnedynsfcd fiefkhdnii mntsqftqss warhvq |
| SseK2 (accession no. CBW18209.1): | |
| (SEQ ID NO: 58) | |
| 1 | marfnaaftr ikimfsrirg liscqsntqt iaptlsppss ghvsfagidy pllplnhqtp |
| 61 | lvfgwfernp drfgqneipi intqknpyln niinaaiiek eriigifvdg dfskgqrkal |
| 121 | gkleqnyrni kviynsdlny smydkkltti ylenitklea qsaserdevl lngvkksled |
| 181 | vlknnpeetl isshnkdkgh lwfdfyrnlf llkgsdafle agkpgchhlq pgggciylda |
| 241 | dmlltdklgt lylpdgiaih vsrkdnhvsl engiiavnrs ehpalikgle imhskpygdp |
| 301 | yndwiskglr hyfdgshiqd ydafcdfief kheniimnts sltasswr |
| SifA (accession no. CBW17257.1): | |
| (SEQ ID NO: 59) | |
| 1 | mpitigngfl kseiltnspr ntkeawwkyl wekikdfffs tykakadrcl hemlfaerap |
| 61 | trerlteiff elkelacasq rdrfqvhnph endatiilri mdqneenell ritqntdtfs |
| 121 | cevmgnlyfl mkdrpdilks hpqmtamikr ryseivdypl pstlclnpag apilsvpldn |
| 181 | iegylytelr kghldgwkaq ekatylaaki qsgiekttri lhhanisest qqnafletma |
| 241 | mcglkqleip pphthipiek mvkevlladk tfqaflvtdp stsqsmlaei veaisdqvfh |
| 301 | aifridpqai qkmaeeqltt lhvrseqqsg clccfl |
| SifB (accession no. CBW17627. 1): | |
| (SEQ ID NO: 60) | |
| 1 | mpitigrgfl ksemfsqsai sqrsfftllw ekikdffcdt qrstadqyik elcdvasppd |
| 61 | aqrlfdlfck lyelsspscr gnfhfqhykd aecqytnlci kdgediplci mirqdhyyye |
| 121 | imnrtvlcvd tqsahlkrys dinikastyv ceplcclfpe rlqlslsggi tfsvdlknie |
| 181 | etliamaekg nlcdwkeqer kaaissrinl giaqagvtai ddaiknkiaa kvientnlkn |
| 241 | aafepnyaqs svtqivyscl fkneilmnml eessshgllc lnelteyvtl qvhnslfsed |
| 301 | lsslvettkn eahhqs |
| PipB (accession no. CBW17123.1): | |
| (SEQ ID NO: 61) | |
| 1 | mpitnaspen ilrylhaagt gtkeamksat sprgilewfv nfftcggvrr snerwfrevi |
| 61 | gklttsllyv nknaffdgnk ifledvngct iclscgaase ntdpmviiev nkngktvtdk |
| 121 | vdserfwnvc rmlklmskhn iqqpdslite dgflnlrgvn lahkdfqged lskidasnad |
| 181 | frettlsnvn lvganlccan lhavnlmgsn mtkanlthad ltcanmsgvn ltaailfgsd |
| 241 | ltdtklngak ldkialtlak altgadltgs qhtptplpdy ndrtlfphpi f |
| PipB2 (accession no. CBW18862.1): | |
| (SEQ ID NO: 62) | |
| 1 | mersldslag maksafgagt saamrqatsp ktileyiinf ftcggirrrn etqyqeliet |
| 61 | maetlkstmp drgaplpeni ilddmdgcrv efnlpgenne agqvivrvsk gdhsetreip |
| 121 | lasfekicra llfrcefslp qdsviltaqg gmnlkgavlt ganltsenlc dadlsganle |
| 181 | gavlfmadce ganfkganls gtslgdsnfk nacledsimc gatldhanlt ganlqhasll |
| 241 | gcsmiecncs ganmdhtnls gatliradms gatlqgatim aaimegavlt ranlrkasfi |
| 301 | stnldgadla eanlnntcfk dctltdlrte datmststqt lfnefyseni |
| SopD2 (accession no. CBW17005.1): | |
| (SEQ ID NO: 63) | |
| 1 | mpvtlsfgnr hnyeinhsrl arlmspdkee alymgvwdrf kdcfrthkkq evlevlytli |
| 61 | hgcerenqae lnvditgmek ihaftqlkey anpsqqdrfv mrfdmnqtqv lfeidgkvid |
| 121 | kcnlhrllnv sencifkvme edeeelflki cikygekisr ypellegfan klkdavnedd |
| 181 | dvkdevyklm rsgedrkmec vewngtltee eknklrclqm gsfnittqff kigywelege |
| 241 | vlfdmvhptl syllqaykps lssdlietnt mlfsdvlnkd yddyqnnkre idailrriyr |
| 301 | shnntlfise ksscrnmli |
| GogB (accession no. CBW18646.2): | |
| (SEQ ID NO: 64) | |
| 1 | mqyaytsnea tsnlellnkw riespdieke ernsiydkii eanhtgslsi tahhvtsipv |
| 61 | fpdniselnl sscytlesip nlpdglkslt isgnqtikis yfpdslesls idmqayeeny |
| 121 | tfpalpyglk sftacygkf1 pplpphlssl slqnfseilc aelpykldkl dlqncpflpl |
| 181 | mkmlpeelke lsielirtvp gtviddilpd klkklsinfc dniklpvklp vnlksinlss |
| 241 | rtpiaweipt cnlpahidis tdgyvklnpe fltrsditfs nkpagdvlsf qpgdvvyglc |
| 301 | kardrvntlv nslyyfskkd iiiqntltda vwdrknravf nkdekiaerl ndvqrgiffr |
| 361 | eflsqhkkyn itedkysdls neecwiktsk aglefqtrlr ersvifvidn lvdaisdian |
| 421 | ktgkhgnsit ahelrwvyrn rhddlvkqnv kfflngeais hedvfslvgw dkykpknrnr |
| SseL (accession no. CBW18358.1): | |
| (SEQ ID NO: 65) | |
| 1 | msdealtllf savengdqnc idllcnlalr nddlghrvek flfdlfsgkr tgssdidkki |
| 61 | nqaclvlhqi annditkdnt ewkklhapsr llymagsatt dlskkigiah kimgdqfaqt |
| 121 | dqeqvgven1 wcgarmlssd elaaatqgly qespllsvny piglihpttk enilstqlle |
| 181 | kiaqsglshn evflvntgdh wllclfykla ekikclifnt yydlnentkq eiieaakiag |
| 241 | isesdevnfi emnlqnnvpn gcglfcyhti qllsnagqnd pattlrefae nfltlsveeg |
| 301 | alfntqtrrq iyeyslq |
| SteC (accession no. CBW17723.1): | |
| (SEQ ID NO: 66) | |
| 1 | mpftfqignh scqiserylr diidnkrehv fstcekfidf frniftrrsl isdyreiynl |
| 61 | lcqkkehpdi kgpfspgpfs krdedctrwr pllgyiklid asrpetidky tvevlahqen |
| 121 | mlllqmfydg vlvtetecse rcvdflketm fnynngeitl aalgndnlpp seagsngiye |
| 181 | afeqrlidfl ttpatasgye sgaidqtdas qpaaieafin spefqknirm rdieknkigs |
| 241 | gsygtvyrlh ddfvykipvn ergikvdvns pehrnchpdr vskylnmand dknfsrsaim |
| 301 | ningkdytvl vskyiqgqef dvedednyrm aeallksrgv ymhdinilgn ilvkegvlff |
| 361 | vdgdqivlsq esrqqrsvsl atrqleeqik ahhmiklkra etegntedve yykslitdld |
| 421 | aligeeeqtp apgrrfklaa peegtlvakv lkdelkk |
| SspH1 (accession no. STM14_1483): | |
| (SEQ ID NO: 67) | |
| 1 | mfnirntqps vsmqalagaa apeaspeeiv wekiqvffpq enyeeaqqcl aelchpargm |
| 61 | lpdhissqfa rlkaltfpaw eeniqcnrdg inqfcildag skeilsitld dagnytvncq |
| 121 | gyseahdfim dtepgeecte faegasgtsl rpattvsqka aeydavwskw erdapagesp |
| 181 | graavvqemr dclnngnpvl nvgasglttl pdrlpphitt lvipdnnlts lpelpeglre |
| 241 | levsgnlqlt slpslpqglq klwaynnrlt slpemspglq eldvshnqlt rlpqsltgls |
| 301 | elrvsgnnlt slpalpsglq klwaynnrlt slpemspglq eldvshnqlt rlpqsltgls |
| 361 | saarvyldgn plsvrtlqal rdiighsgir ihfdmagpsv prearalhla vadwltsare |
| 421 | geaaqadrwq afglednaaa fslvidrlre tenfkkdagf kaqisswltq laedaalrak |
| 481 | tfamateats tcedrvthal hqmnnvqlvh naekgeydnn lqglvstgre mfrlatleqi |
| 541 | arekagtlal vddvevylaf qnklkeslel tsvtsemrff dvsgvtvsdl qaaelqvkta |
| 601 | ensgfskwil qwgplhsvle rkvperfnal rekqisdyed tyrklydevl kssglvddtd |
| 661 | aertigvsam dsakkefldg lralvdevlg syltarwrln |
| SspH2 (accession no. CBW18313.1): | |
| (SEQ ID NO: 68) | |
| 1 | mpfhigsgcl patisnrriy riawsdtppe msswekmkef fcsthqteal eciwtichpp |
| 61 | agttredvin rfellrtlay agweesihsg qhgenyfcil dedsqeilsv tlddagnytv |
| 121 | ncqgysethr ltldtaqgee gtghaegasg tfrtsflpat tapqtpaeyd avwsawrraa |
| 181 | paeesrgraa vvqkmracln ngnavlnvge sglttlpdcl pahittlvip dnnltslpal |
| 241 | ppelrtlevs gnqltslpvl ppgllelsif snplthlpal psglcklwif gnqltslpvl |
| 301 | ppglqelsvs dnqlaslpal pselcklway nnqltslpml psglqelsvs dnqlaslptl |
| 361 | pselyklway nnrltslpal psglkelivs gnrltslpvl pselkelmvs gnrltslpml |
| 421 | psgllslsvy rnqltrlpes lihlssettv nlegnplser tlqalreits apgysgpiir |
| 481 | fdmagasapr etralhlaaa dwlvparege papadrwhmf gqednadafs lfldrlsete |
| 541 | nfikdagfka qisswlagla edealrantf amateatssc edrvtfflhq mknvqlvhna |
| 601 | ekgqydndla alvatgremf rlgkleqiar ekvrtlalvd eievwlayqn klkkslglts |
| 661 | vtsemrffdv sgvtvtdqad aelqvkaaek sefrewilqw gplhrvlerk apervnalre |
| 721 | kqisdyeety rmlsdtelrp sglvgntdae rtigarames akktfldglr plveemlgsy |
| 781 | lnvqwrrn |

In one embodiment, the Salmonella gene under the regulation of an inducible promoter is selected from ftsW (accession no. CBW16230.1), ftsA (accession no. CBW16235.1), ftsZ (accession no. CBW16236.1), murE (accession no. CBW16226.1), mukF (accession no. CBW17025.1), imp (accession no. CBW16196.1), secF (accession no. CBW16503.1), eno (accession no. CBW19030.1), hemH (accession no. CBW16582.1), tmk (accession no. CBW17233.1), dxs (accession no. CBW16516.1), uppS (accession no. CBW16324.1), cdsA (accession no. CBW16325.1), accA (accession no. CBW16335.1), pssA (accession no. CBW18718.1), msbA (accession no. CBW17017.1), tsf (accession no. CBW16320.1), trmD) (accession no. CBW18749.1), cca (accession no. CBW19276.1), infB (accession no. CBW19355.1), rpoA (accession no. CBW19477.1), rpoB (accession no. CBW20180.1), rpoC (accession no. CBW20181.1), holA (accession no. CBW16734.1), dnaC (accession no. CBW20563.1), or eng (EngA accession no. CBW18582.1; EngB accession no. CBW20039.1).

| ftsW (accession no. CBW16230.1): | |
|---|---|
| (SEQ ID NO: 69) | |
| 1 | mmasrdkdad slimydrtll wltfglaaig fvmvtsasmp vgqrlandpf lfakrdalyi |
| 61 | flafclamvt lrlpmtfwqk ysttmliasi imllivlvvg ssvngasrwi algplriqpa |
| 121 | eftklslfcy lanylvrkvd evrnnlrgfl kpmgvilvla vlllaqpdlg tvvvlfvttl |
| 181 | amlflagakl wqfiaiigmg isavillila epyrirrvts fwnpwedpfg sgyqltqslm |
| 241 | afgrgeiwgq glgnsvqkle ylpeahtdfi faiigeelgy igvvlallmv ffvaframsi |
| 301 | grkaleidhr fsgflacsig iwfsfqalvn vgaaagmlpt kgltlplisy ggssllimst |
| 361 | aimfllridy etrlekaqaf trgsr |
| ftsA (accession no. CBW16235.1): | |
| (SEQ ID NO: 70) | |
| 1 | mikatdrklv vgleigtakv aalvgevlpd gmvniigvgs cpsrgmdkgg vndlesvvkc |
| 61 | vqraidqael madcqissvy lalsgkhisc qneigmvpis eeevtqedve nvvhtaksvr |
| 121 | vrdehrvlhv ipqeyaidyq egiknpvgls gvrmqakvhl itchndmakn ivkavercgl |
| 181 | kvdqlifagl aasysvlted erelgvcvvd igggtmdiav ytggalrhtk vipyagnvvt |
| 241 | sdiayafgtp psdaeaikvr hgcalgsivg kdesvevpsv ggrpprslqr qtlaeviepr |
| 301 | ytellnlvne eilqlqeqlr qqgvkhhlaa givitggaaq ieglaacaqr vfhtqvriga |
| 361 | plnitgltdy agepyystav gllhygkesh lngeaevekr vtasvgswik rlnswlrkef |
| ftsZ (accession no. CBW16236.1): | |
| (SEQ ID NO: 71) | |
| 1 | mfepmeltnd avikvigvgg gggnavehmv reriegveff avntdaqalr ktavgqtiqi |
| 61 | gsgitkglga ganpevgrna adedrealra alegadmvfi aagmgggtgt gaapvvaeva |
| 121 | kdlgiltvav vtkpfnfegk krmafaeqgi telskhvdsl itipndklkk vlgrgislld |
| 181 | afgaandvlk gavqgiaeli trpglmnvdf advrtymsem gyammgsgva sgedraeeaa |
| 241 | emaissplle didlsgargv lvnitagfdl rldefetvgn tirafasdna tvvigtsldp |
| 301 | dmndelrvtv vatgigmdkr peitlvtnkq vqqpvldryq qhgmapltqe qktvakvvnd |
| 361 | ntpqaakepd yldipaflrk qad |
| murE (accession no. CBW16226.1): | |
| (SEQ ID NO: 72) | |
| 1 | madrnlrdll apwvaglpar elremtldsr vaaagdlfva vvghqadgrr yipqaiaqgv |
| 61 | aaiiaeakde asdgeiremh gvpvvylsql nerlsalagr fyhepsenmr lvavtgtngk |
| 121 | ttttqllaqw sqllgetsav mgtvgngllg kviptenttg savdvqhvla slvaqgatfg |
| 181 | amevsshglv qhrvaalkfa asvftnlsrd hldyhgdmah yeaakwmlys thhhgqaivn |
| 241 | addevgrrwl aslpdavavs meghinpnch grwlkaeave yhdrgatirf asswgegeie |
| 301 | srlmgafnvs nlllalatll algypltdll ktaarlqpvc grmevftapg kptvvvdyah |
| 361 | tpdalekalq aarlhcagkl wcvfgcggdr dkgkrplmga iaeefadivv vtddnprtee |
| 421 | praiindila gmldagqvrv megraeavtn aimqakdndv vliagkghed yqivgtqrld |
| 481 | ysdrvtaarl lgvia |
| mukF (accession no. CBW17025.1): | |
| (SEQ ID NO: 73) | |
| 1 | msefsqtvpe lvawarkndf sislpvdrls fllavatlng erldgemseg elvdafrhvs |
| 61 | dafeqtseti gvrannaind mvrqrllnrf tseqaegnai yrltplgigi tdyyirqref |
| 121 | stlrlsmqls ivagelkraa daaaeggdef hwhrnvyapl kysvaeifds idltqrimde |
| 181 | qqqqvkddia qllnkdwraa isscelllse tsgtlrelqd tleaagdklq anllriqdat |
| 241 | mthddlhfvd rlvfdlqskl driiswgqqs idlwigydrh vhkfirtaid mdknrvfaqr |
| 301 | lrqsvqtyfd dpwaltyana drlldmrdee malrddevtg elppdleyee fneireqlaa |
| 361 | iieeqlaiyk trqtpldlgl vvreylaqyp rarhfdvari vidqavrlgv aqadftglpa |
| 421 | kwqpindyga kvqahvidky |
| imp (accession no. CBW16196.1): | |
| (SEQ ID NO: 74) | |
| 1 | mkkriptlla tmiasalysh qglaadlasq cmlgypsydr plvkgdtndl pvtinadnak |
| 61 | gnypddavft gnvdimqgns rlqadevqlh qkqaegqpep vrtvdalgnv hyddnqvilk |
| 121 | gpkgwanlnt kdtnvwegdy qmvgrqgrgk adlmkqrgen rytilengsf tsclpgsdtw |
| 181 | svvgsevihd reeqvaeiwn arfkvgpvpi fyspylqlpv gdkrrsgfli pnakyttkny |
| 241 | fefylpyywn iapnmdatit phymhrrgni mwenefrylt qagegvmeld ylpsdkvyed |
| 301 | dhpkegdkhr wlfnwghsgv mdqvwrfnvd ytkvsdssyf ndfdskygss tdgyatqkfs |
| 361 | vgyavqnfda tvstkqfqvf ndqntssysa epqldvnyyh ndlgpfdtri ygqavhfvnt |
| 421 | kdnmpeatrv hleptinlpl snrwgslnte aklmathyqq tnldsynsdp nnknkledsv |
| 481 | nrvmpqfkvd gkliferdma mlapgytqtl eprvqylyvp yrdqsgiyny dssllqsdyn |
| 541 | qlfrdrtygg ldriasanqv ttgvttriyd daaverfnvs vgqiyyftes rtgddnikwe |
| 601 | nddktgslvw agdtywrise rwglrsgvqy dtrldsvats sssleyrrdq drlvqlnyry |
| 661 | aspeyiqatl psyystaeqy knginqvgav aswpiadrws ivgayyfdtn sskpadqmlg |
| 721 | lqynsccyai rvgyerklng wdndkqhaiy dnaigfniel rglssnyglg tqemlrsnil |
| 781 | pyqssm |
| secF (accession no. CBW16503.1): | |
| (SEQ ID NO: 75) | |
| 1 | maqeytveql nhgrkvydfm rwdfwafgis gllliaaivi mgvrgfnwgl dftggtviei |
| 61 | tlekpaemdv mrealqkagy eepqlqnfgs shdimvrmpp tegetggqvl gskvvtiine |
| 121 | atnqnaavkr iefvgpsvga dlaqtgamal lvalisilvy vgfrfewrla agvvialahd |
| 181 | viitlgilsl fhieidltiv aslmsvigys lndsivvsdr irenfrkirr gtpyeifnvs |
| 241 | ltqtlhrtli tsgttlvvil mlylfggpvl egfsltmlig vsigtassiy vasalalklg |
| 301 | mkrehmlqqk vekegadqps ilp |
| eno (accession no. CBW19030.1): | |
| (SEQ ID NO: 76) | |
| 1 | mskivkvigr eiidsrgnpt veaevhlegg fvgmaaapsg astgsreale lrdgdksrfl |
| 61 | gkgvtkavga vngpiaqail gkdakdqagi dkimidldgt enksnfgana ilavslanak |
| 121 | aaaaakgmpl yehiaelngt pgkysmpvpm mniinggeha dnnvdiqefm iqpvgaktvk |
| 181 | eairmgsevf hhlakvlkgk gmntavgdeg gyapnlgsna ealaviaeav kaagyelgkd |
| 241 | itlamdcaas efykdgkyvl agegnkafts eefthfleel tkqypivsie dgldesdwdg |
| 301 | fayqtkvlgd kiqlvgddlf vtntkilkeg iekgiansil ikfnqigslt etlaaikmak |
| 361 | dagytavish rsgetedati adlavgtaag qiktgsmsrs drvakynqli rieealgeka |
| 421 | pyngrkeikg ga |
| hemH (accession no. CBW16582.1): | |
| (SEQ ID NO: 77) | |
| 1 | mrqtktgill anlgtpdapt peavkrylkq flsdrrvvdt prllwwpllr gvilplrspr |
| 61 | vaklyqsiwm dggsplmvys reqqqalaar lpdtpvalgm sygspslesa vdellasdvd |
| 121 | hivviplypq yscstvgavw delgrilark rripgisfir dyaddgayid alaksaresf |
| 181 | arhgepdvll lsyhgipgry adegddypqr crdttrelvs alglppekvm mtfqsrfgre |
| 241 | pwltpytdet lkmlgekgtg hiqvmcpgfa adcletleei aeqnreifle aggkkyayip |
| 301 | alnatpehid mmlkltapyr |
| tmk (accession no. CBW17233.1): | |
| (SEQ ID NO: 78) | |
| 1 | mgsnyivieg legagkttar dvvvetleql girnmiftre pggtqlaekl rslvldirsv |
| 61 | gdevitdkae vlmfyaarvq lvetvikpal aqgvwvigdr hdlstqayqg ggrgidqtml |
| 121 | atlrdavlgd frpdltlyld vtpevglkra rargdldrie qesfdffnrt rarylelaaq |
| 181 | dsrirtidat qpldavmrdi ratvtkwvqe qaa |
| dxs (accession no. CBW16516, 1): | |
| (SEQ ID NO: 79) | |
| 1 | msfdiakypt lalvdstqel rllpkeslpk lcdelrryll dsvsrssghf asglgtvelt |
| 61 | valhyvyntp fdqliwdvgh qayphkiltg rrdkigtirq kgglhpfpwr geseydvlsv |
| 121 | ghsstsisag igiavaaeke gkdrrtvcvi gdgaitagma feamnhagdi rpdmlvilnd |
| 181 | nemsisenvg alnnhlaqll sgklysslre ggkkvfsgvp pikellkrte ehikgmvvpg |
| 241 | tlfeelgfny igpvdghdvm glistlknmr dlkgpqflhi mtkkgrgyep aekdpitfha |
| 301 | vpkfdpssgc lpkssgglpg yskifgdwlc etaakdsklm aitpamregs gmvefsrkfp |
| 361 | dryfdvaiae qhavtfaagl aiggykpvva iystflqray dqvihdvaiq klpvmfaidr |
| 421 | agivgadgqt hqgafdlsyl rcipdmvimt psdenecrqm lftgyhyndg ptavryprgn |
| 481 | aqgvaltple klpigkglvk rhgeklailn fgtlmpeaak vaealnatlv dmrfvkpldd |
| 541 | tlilemaaqh dalvtleena imggagsgvn evlmahrkpv pvlniglpdf fipqgtqeea |
| 601 | raelgldaag ieakikawla |
| uppS (accession no. CBW16324.1): | |
| (SEQ ID NO: 80) | |
| 1 | mlsatqpvse nlpahgcrhv aiimdgngrw akkqgkiraf ghkagaksvr ravsfaanng |
| 61 | idaltlyafs senwnrpaqe vsalmelfvw aldsevkslh rhnvrlriig disrfnsrlq |
| 121 | erirksealt ahntgltlni aanyggrwdi vqgvrqlaeq vqagvlrpdq ideerlgqqi |
| 181 | cmhelapvdl virtggehri snfllwqiay aelyftdvlw pdfdeqdfeg alhafanrer |
| 241 | rfggtepgdd ka |
| cdsA (accession no. CBW16325.1): | |
| (SEQ ID NO: 81) | |
| 1 | mlkyrlisaf vlipaviaal fllppvgfai itlvvcmlaa wewgqlsgfa arsqrvwlav |
| 61 | lcglllalml fllpeyhhni rqplvemslw aslgwwvval llvlfypgsa aiwrnsktlr |
| 121 | lifglltivp ffwgmlalra whydenhysg aiwllyvmil vwgadsgaym fgklfgkhkl |
| 181 | apkvspgktw qgfigglata aviswgygmw anlnvapvil licsvvaala svlgdltesm |
| 241 | fkreagikds ghlipghggi ldridsltaa vpvfacllll vfrtl |
| accA (accession no. CBW16335.1): | |
| (SEQ ID NO: 82) | |
| 1 | mslnfldfeq piaeleakid sltavsrqde kldinideev hrlreksvel trkifadlga |
| 61 | wqvaqlarhp qrpytldyvr lafdefdela gdrayaddka ivggiarleg rpvmiighqk |
| 121 | gretkekirr nfgmpapegy rkalrlmema erfnmpiitf idtpgaypgv gaeergqsea |
| 181 | iarnlremsr lnvpvictvi geggsggala igvgdkvnml qystysvisp egcasilwks |
| 241 | adkaplaaea mgiiaprlke lklidsiipe plggahrnpe amaaslkaql ledladldvl |
| 301 | stddlknrry qrlmsygya |
| pssA (accession no. CBW18718.1): | |
| (SEQ ID NO: 83) | |
| 1 | mlskfkrnkh qqhlaqlpki sqsvddvdff ytpatfretl lekiasatqr icivalyleq |
| 61 | ddggkgilda lyaakrqrpe ldvrvlvdwh raqrgrigaa asntnadwyc rlaqenpgid |
| 121 | vpvygvpint realgvlhfk gfiiddsvly sgaslndvyl hqhdkyrydr yqlirnrqma |
| 181 | dimfdwvtqn lmngrgvnrl dntqrpkspe ikndirlyrq elrdasyhfq gdandeqlsv |
| 241 | tplvglgkss llnktifhlm pcaehkltic tpyfnlpavl vrniiqllrd gkkveiivgd |
| 301 | ktandfyipe depfkiigal pylyeinlrr flsrlqyyvn tdqlvvrlwk dddntyhlkg |
| 361 | mwvddkwmll tgnnlnpraw rldlenaili hdpkqelapq rekelelirt httivkhyrd |
| 421 | lqsiadypik vrklirrlrr iridrlisri l |
| msbA (accession no. CBW17017.1): | |
| (SEQ ID NO: 84) | |
| 1 | mhndkdlstw qtfrrlwpti apfkagliva gialilnaas dtfmlsllkp llddgfgktd |
| 61 | rsvllwmplv viglmilrgi tsyissycis wvsgkvvmtm rrrlfghmmg mpvaffdkqs |
| 121 | tgtllsrity dseqvassss galitvvreg asiiglfimm fyyswqlsii lvvlapivsi |
| 181 | airvvskrfr sisknmqntm gqvttsaeqm lkghkevlif ggqevetkrf dkvsnkmrlq |
| 241 | gmkmvsassi sdpiiqlias lalafvlyaa sfpsvmdslt agtitvviss mialmrplks |
| 301 | ltnvnaqfqr gmaacqtlfa ildseqekde gkrvidratg dlefrnvtft ypgrevpalr |
| 361 | ninlkipagk tvalvgrsgs gkstiaslit rfydideghi lmdghdlrey tlaslrnqva |
| 421 | lvsqnvhlfn dtvanniaya rteeysreqi eeaarmayam dfinkmdngl dtiigengvl |
| 481 | lsggqrqria iarallrdsp ilildeatsa ldteseraiq aaldelqknr tslviahrls |
| 541 | tieqadeivv vedgiiverg thsellaqhg vyaqlhkmqf gq |
| tsf (accession no. CBW16320.1): | |
| (SEQ ID NO: 85) | |
| 1 | maeitaslvk elrertgagm mdckkaltea ngdielaien mrksgaikaa kkagnvaadg |
| 61 | viktkidgnv afilevncqt dfvakdagfq afadkyldaa vagkitdvev lkaqfeeerv |
| 121 | alvakigeni nirrvasleg dvlgsyqhga rigvlvaakg adeelvkqla mhvaaskpef |
| 181 | vkpedvsadv vekeyqvqld iamqsgkpke iaekmvegrm kkftgevslt gqpfvmepsk |
| 241 | svgqllkehn advtgfirfe vgegiekvet dfaaevaams kqs |
| trmD (accession no. CBW18749.1): | |
| (SEQ ID NO: 86) | |
| 1 | mfigivslfp emfraitdyg vtgravkkgl lniqswsprd fahdrhrtvd drpygggpgm |
| 61 | lmmvqplrda ihaskaaage gakviylspq grkldqagvs elatnqklil vcgryegvde |
| 121 | rviqteidee wsigdyvlsg gelpamtlid svarfipgvl gheasaieds fadglldcph |
| 181 | ytrpevlegm evppvllsgn haeirrwrlk qslgrtwlrr pellenlalt eeqarllaef |
| 241 | ktehaqqqhk hdgma |
| cca (accession no. CBW19276.1): | |
| (SEQ ID NO: 87) | |
| 1 | mkiylvggav rdallglpvk dkdwvvvgat pqemldagyq qvgrdfpvfl hpqtheeyal |
| 61 | arterksgsg ytgftcyaap dvtleadlqr rditinalar ddagqiidpy hgrrdlearl |
| 121 | lrhvspafge dplrvlrvar faaryahlsf riadetlalm remtaagele hltpervwke |
| 181 | tenalttrnp qvyfqvlrdc galrvlfpei dalfgypapa kwhpeidtgv htlmtlsmaa |
| 241 | mlspqldvrf atlchdlgkg ltpknlwprh hghgpagvkl veqlcqrlrv pndlrdlakl |
| 301 | vaeyhdliht fpilqpktiv klidaidawr kpqrveqial tseadvrgrt gfeasdypqg |
| 361 | rwlreawqva qavptkevve agfkgieire eltkrrlaav anwkekrcpn pas |
| infB (accession no. CBW19355.1): | |
| (SEQ ID NO: 88) | |
| 1 | mtdvtlkala aerqvsvdrl vqqfadagir ksaddsvsaq ekqtllahln reavsgpdkl |
| 61 | tlqrktrstl nipgtggksk svqievrkkr tfvkrdpqea erlaaeeqaq reaeeqarre |
| 121 | aeeqakreaq qkaereaaeq akreaaekak reaaekdkvs nqqtddmtkt aqaekarren |
| 181 | eaaelkrkae eearrkleee arrvaeearr maeenkwtat pepvedtsdy hvttsqharq |
| 241 | aedendreve ggrgrgrnak aarpakkgkh aeskadreea raavrggkgg krkgsslqqg |
| 301 | fqkpaqavnr dvvigetitv gelankmavk gsqvikammk lgamatinqv idqetaqlva |
| 361 | eemghkvilr reneleeavm sdrdtgaaae prapvvtimg hvdhgktsll dyirstkvas |
| 421 | geaggitqhi gayhvetdng mitfldtpgh aaftsmrarg agatdivvlv vaaddgvmpq |
| 481 | tieaiqhaka agvpvvvavn kidkpeadpd rvknelsqyg ilpeewgges qfvhvsakag |
| 541 | tgidelldai llqaevlelk avrkgmasga viesfldkgr gpvatvlvre gtlhkgdivl |
| 601 | cgfeygrvra mrnelgqevl eagpsipvei lglsgvpaag devtvvrdek karevalyrq |
| 661 | gkfrevklar qqksklenmf anmtegevhe vnivlkadvq gsveaisdsl lklstdevkv |
| 721 | kiigsgvggi tetdatlaaa snailvgfnv radasarkvi esesldlryy sviynlidev |
| 781 | kaamsgmlsp elkqqiigla evrdvfkspk fgaiagcmvt egtikrhnpi rvlrdnvviy |
| 841 | egeleslrrf kddvnevrng mecgigvkny ndvrvgdmie vfeiieiqrt ia |
| rpoA (accession no. CBW19477.1): | |
| (SEQ ID NO: 89) | |
| 1 | mqgsvteflk prlvdieqvs sthakvtlep lergfghtlg nalrrillss mpgcavteve |
| 61 | idgvlheyst kegvqedile illnlkglav rvqgkdevil tlnksglgpv taadithdgd |
| 121 | veivkpqhvi chltdenasi smrikvqrgr gyvpastrih seederpigr llvdacyspv |
| 181 | eriaynveaa rveqrtdldk lviemetngt idpeeairra atilaeqlea fvdlrdvrqp |
| 241 | evkeekpefd pillrpvddl eltvrsancl kaeaihyigd lvqrtevell ktpnlgkksl |
| 301 | teikdvlasr glslgmrlen wppasiade |
| rpoB (accession no. CBW20180.1): | |
| (SEQ ID NO: 90) | |
| 1 | mvysytekkr irkdfgkrpq vldvpyllsi qldsfqkfie qdpegqygle aafrsvfpiq |
| 61 | sysgnselqy vsyrlgepvf dvqecqirgv tysaplrvkl rlviyereap egtvkdikeq |
| 121 | evymgeiplm tdngtfving tervivsqlh rspgvffdsd kgkthssgkv lynariipyr |
| 181 | gswldfefdp kdnlfvridr rrklpatiil ralnytteqi ldlffekvvf eirdnklqme |
| 241 | liperlrget asfdieangk vyvekgrrit arhirqlekd dikhievpve yiagkvvskd |
| 301 | yvdestgeli caanmelsld llaklsqsgh krietlftnd ldhgpyiset vrvdptndrl |
| 361 | salveiyrmm rpgepptrea aeslfenlff sedrydlsav grmkfnrsil rdeiegsgil |
| 421 | skddiidvmk klidirngkg evddidhlgn rrirsvgema enqfrvglvr veravkerls |
| 481 | lgdldtlmpq dminakpisa avkeffgssq lsqfmdqnnp lseithkrri salgpggltr |
| 541 | eragfevrdv hpthygrvcp ietpegpnig linslsvyag tneygfletp yrrvvdgvvt |
| 601 | deihylsaie egnyviagqn snlddeghfv edlvtcrskg esslfsrdqv dymdvstqqv |
| 661 | vsvgaslipf lehddanral mganmqrqav ptlradkplv gtgmeravav dsgvtavakr |
| 721 | ggtvqyvdas rivikvnede mypgeagidi ynltkytrsn qntcinqmpc vslgepverg |
| 781 | dvladgpstd lgelalgqnm rvafmpwngy nfedsilvse rvvqedrftt ihiqelacvs |
| 841 | rdtklgpeel tadipnvgea alskldesgi vyigaevtgg dilvgkvtpk getqltpeek |
| 901 | llraifgeka sdvkdsslrv pngvsgtvid vqvftrdgve kdkraleiee mqlkqakkdl |
| 961 | seelqileag lfsriravlv ssgveaekld klprdrwlel gltdeekqnq leqlaeqyde |
| 1021 | lkhefekkle akrrkitqgd dlapgvlkiv kvylavkrri qpgdkmagrh gnkgviskin |
| 1081 | piedmpyden gtpvdivlnp lgvpsrmnig qilethlgma akgigdkina mlkqqqevak |
| 1141 | lrefiqrayd lgadvrqkvd lstfsddevl rlaenlrkgm piatpvfdga keaeikellk |
| 1201 | lgdlptsgqi tlfdgrtgeq ferpvtvgym ymlklnhlvd dkmharstgs yslvtqqplg |
| 1261 | gkaqfggqrf gemevwalea ygaaytlqem ltvksddvng rtkmyknivd gnhqmepgmp |
| 1321 | esfnvllkei rslginiele de |
| rpoC (accession no. CBW20181.1): | |
| (SEQ ID NO: 91) | |
| 1 | mkdllkflka qtkteefdai kialaspdmi rswsfgevkk petinyrtfk perdglfcar |
| 61 | ifgpvkdyec lcgkykrlkh rgvicekcgv evtqtkyrre rmghielasp tahiwflksl |
| 121 | psrigllldm plrdiervly fesyvviegg mtnlerqqil teeqyldale efgdefdakm |
| 181 | gaeaiqallk smdleqecet lreelnetns etkrkkltkr iklleafvqs gnkpewmilt |
| 241 | vlpvlppdlr plvpldggrf atsdlndlyr rvininnrlk rlldlaapdi ivrnekrmlq |
| 301 | eavdalldng rrgraitgsn krplksladm ikgkqgrfrq nllgkrvdys grsvitvgpy |
| 361 | lrlhqcglpk kmalelfkpf iygklelrgl attikaakkm vereeavvwd ildevirehp |
| 421 | vllnraptlh rlgiqafepv liegkaiqlh plvcaaynad fdgdqmavhv pltleaqlea |
| 481 | ralmmstnni lspangepii vpsqdvvlgl yymtrdcvna kgegmvltgp keaeriyrag |
| 541 | laslharvkv riteyekden gefvahtslk dttvgrailw mivpkglpfs ivnqalgkka |
| 601 | iskmlntcyr ilglkptvif adqtmytgfa yaarsgasvg iddmvipekk heiiseaeae |
| 661 | vaeiqeqfqs glvtageryn kvidiwaaan drvskammdn lqtetvinrd gqeeqqvsfn |
| 721 | siymmadsga rgsaaqirql agmrglmakp dgsiietpit anfreglnvl qyfisthgar |
| 781 | kgladtalkt ansgyltrrl vdvaqdlvvt eddcgthegi lmtpvieggd vkeplrdrvl |
| 841 | grvtaedvlk pgtadilvpr ntllheqwed lleansvdav kvrsvvscdt dfgvcahcyg |
| 901 | rdlarghiin kgeaigviaa qsigepgtql tmrtfhigga asraaaessi qvknkgsikl |
| 961 | snvksvvnss gklvitsrnt elklidefgr tkesykvpyg avmakgdgeq vaggetvanw |
| 1021 | dphtmpvite vsgfirftdm idgqtitrqt deltglsslv vldsaerttg gkdlrpalki |
| 1081 | vdaqgndvli pgtdmpaqyf lpgkaivqle dgvqissgdt laripqesgg tkditgglpr |
| 1141 | vadlfearrp kepailaeia givsigketk gkrrlvitpv dgsdpyeemi pkwrqlnvfe |
| 1201 | gervergdvi sdgpeaphdi lrlrgvhavt ryivnevqdv yrlqgvkind khievivrqm |
| 1261 | lrkatiesag ssdflegeqv eysrvkianr eleangkvga tfsrdllgit kaslatesfi |
| 1321 | saasfqettr viteaavagk rdelrglken vivgrlipag tgyayhqdrm rrraageqpa |
| 1381 | tpqvtaedas aslaellnag lggsdne |
| holA (accession no. CBW16734.1): | |
| (SEQ ID NO: 92) | |
| 1 | mirlypeqlr aqlneglraa ylllgndpll lqesqdairl aaasqgfeeh haftldpstd |
| 61 | wgslfslcqa mslfasrqtl vlqlpengpn aamneqlatl sellhddlll ivrgnkltka |
| 121 | qenaawytal adrsvqvscq tpeqaqlprw vaarakaqnl qlddaanqll cycyegnlla |
| 181 | laqalerlsl lwpdgkltlp rveqavndaa hftpfhwvda llmgkskral hilqqlrleg |
| 241 | sepvillrtl qrellllvnl krqsahtplr alfdkhrvwq nrrpmigdal qrlhpaqlrq |
| 301 | avqlltrtei tlkqdygqsv wadleglsll lchkaladvf idg |
| dnaC (accession no. CBW20563.1): | |
| (SEQ ID NO: 93) | |
| 1 | mknvgdlmqr lqkmmpahit pafktgeell awqkeqgeir aaalarenra mkmqrtfnrs |
| 61 | girplhqncs fdnyrvecdg qmnalskarq yvdefdgnia sfvfsgkpgt gknhlaaaic |
| 121 | nelllrgksv liitvadims amkdtfsnre tseeqllndl snvdllvide igvqtesrye |
| 181 | kviinqivdr rssskrptgm ltnsnmeemt kmlgervmdr mrlgnslwvn ftwdsyrsrv |
| 241 | tgkey |
| eng (EngA accession no. CBW18582.1): | |
| (SEQ ID NO: 94) | |
| 1 | mvpvvalvgr pnvgkstlfn rltrtrdalv adfpgltrdr kygraevegr eficidtggi |
| 61 | dgtedgvetr maeqsllaie eadvvlfmvd araglmpade aiakhlrsre kptflvankt |
| 121 | dgldpdqavv dfyslglgei ypiaashgrg vlsllehvll pwmddvapqe evdedaeywa |
| 181 | qfeaeqngee apeddfdpqs lpiklaivgr pnvgkstltn rilgeervvv ydmpgttrds |
| 241 | iyipmerder eyvlidtagv rkrgkitdav ekfsviktlq aiedanvvll vidaregisd |
| 301 | qdlsllgfil nsgrslvivv nkwdglsqev keqvketldf rlgfidfarv hfisalhgsg |
| 361 | vgnlfesvre aydsstrrvs tamltrimtm avedhqpplv rgrrvklkya haggynppiv |
| 421 | vihgnqvkdl pdsykrylmn yfrkslevmg tpiriqfkeg enpyankrnt ltptqmrkrk |
| 481 | rlmkhikksk |
| EngB (accession no. CBW20039.1): | |
| (SEQ ID NO: 95) | |
| 1 | mmsapdirhl psdcgievaf agrsnagkss alntltnqks lartsktpgr tqlinlfevv |
| 61 | dgkrlvdlpg ygyaevpeem krkwqralge ylekrqslqg lvvlmdirhp lkdldqqmiq |
| 121 | wavesniqvl vlltkadkla sgarkaqlnm vreavlafng dvqveafssl kkqgvdklrq |
| 181 | kldswfsela pveeiqdge |

Other inducible promotors for use in the invention, including to inducibly control flagella, include, but are not limited to:

pbad sequences
Full PBAD sequence with araC repressor (from
Invitrogen pbad-his-myc A plasmid)
(SEQ ID NO: 96)
ttatgacaacttgacggctacatcattcactttttcttcacaaccggcac
ggaactcgctcgggctggccccggtgcattttttaaatacccgcgagaaa
tagagttgatcgtcaaaaccaacattgcgaccgacggtggcgataggcat
ccgggtggtgctcaaaagcagcttcgcctggctgatacgttggtcctcgc
gccagcttaagacgctaatccctaactgctggcggaaaagatgtgacaga
cgcgacggcgacaagcaaacatgctgtgcgacgctggcgatatcaaaatt
gctgtctgccaggtgatcgctgatgtactgacaagcctcgcgtacccgat
tatccatcggtggatggagcgactcgttaatcgcttccatgcgccgcagt
aacaattgctcaagcagatttatcgccagcagctccgaatagcgcccttc
cccttgcccggcgttaatgatttgcccaaacaggtcgctgaaatgcggct
ggtgcgcttcatccgggcgaaagaaccccgtattggcaaatattgacggc
cagttaagccattcatgccagtaggcgcgcggacgaaagtaaacccactg
gtgataccattcgcgagcctccggatgacgaccgtagtgatgaatctctc
ctggcgggaacagcaaaatatcacccggtcggcaaacaaattctcgtccc
tgatttttcaccaccccctgaccgcgaatggtgagattgagaatataacc
tttcattcccagcggtcggtcgataaaaaaatcgagataaccgttggcct
caatcggcgttaaacccgccaccagatgggcattaaacgagtatcccggc
agcaggggatcattttgcgcttcagccatacttttcatactcccgccatt
cagagaagaaaccaattgtccatattgcatcagacattgccgtcactgcg
tcttttactggctcttctcgctaaccaaaccggtaaccccgcttattaaa
agcattctgtaacaaagcgggaccaaagccatgacaaaaacgcgtaacaa
aagtgtctataatcacggcagaaaagtccacattgattatttgcacggcg
tcacactttgctatqccatagcatttttatccataagattagcggatcct
acctgacgctttttatcgcaactctctactgtttctccatacccgttttt
tgggctaacaggaggaattaacc
PBAD promoter sequence
(SEQ ID NO: 97)
aagaaaccaattgtccatattgcatcagacattgccgtcactgcgtcttt
tactggctcttctcgctaaccaaaccggtaaccccgcttattaaaagcat
tctgtaacaaagcgggaccaaagccatgacaaaaacgcgtaacaaaagtg
tctataatcacggcagaaaagtccacattgattatttgcacggcgtcaca
ctttgctatgccatagcatttttatccataagattagcggatcctacctg
acgctttttatcgcaactctctactgtttctccatacccgttttttgggc
taacaggaggaattaacc
AraC repressor protein
(SEQ ID NO: 98)
atggctgaagcgcaaaatgatcccctgctgccgggatactcgtttaatgc
ccatctggtggcgggtttaacgccgattgaggccaacggttatctcgatt
tttttatcgaccgaccgctgggaatgaaaggttatattctcaatctcacc
attcgcggtcagggggtggtgaaaaatcagggacgagaatttgtttgccg
accgggtgatattttgctgttcccgccaggagagattcatcactacggtc
gtcatccggaggctcgcgaatggtatcaccagtgggtttactttcgtccg
cgcgcctactggcatgaatggcttaactggccgtcaatatttgccaatac
ggggttctttcgcccggatgaagcgcaccagccqcatttcagcgacctgt
ttgggcaaatcattaacgccgggcaaggggaagggcgctattcggagctg
ctggcgataaatctgcttgagcaattgttactgcggcgcatggaagcgat
taacgagtcgctccatccaccgatggataatcgggtacgcgaggcttgtc
agtacatcagcgatcacctggcagacagcaattttgatatcgccagcgtc
gcacagcatgtttgcttgtcgccgtcgcgtctgtcacatcttttccgcca
gcagttagggattagcgtcttaagctggcgcgaggaccaacgtatcagcc
aggcgaagctgcttttgagcaccacccggatgcctatcgccaccgtcggt
cgcaatgttggttttgacgatcaactctatttctcgcgggtatttaaaaa
atgcaccggggccagcccgagcgagttccgtgccggttgtgaagaaaaag
tgaatgatgtagccgtcaagttgtcataa
AraC protein sequence
(SEQ ID NO: 99)
MAEAQNDPLLPGYSFNAHLVAGLIPIEANGYLDFFIDRPLGMKGYILNLT
IRGQGVVKNQGREFVCRPGDILLFPPGEIHHYGRAPEAREWYHQWVYFRP
RAYWHEWLNWPSIFANTGFFRPDEAHQPHFSDLFGQIINAGQGEGRYSEL
LAINLLEQLLLRRMEAINESLHPPMDNRVREACQYISDHLADSNFDIASV
AQHVCLSPSRLSHLFRQQLGISVLSWREDQRISQAKLLLSTTRMPIATVG
RNVGFDDQLYFSRVFKKCTGASPSEFRAGCEEKVNDVAVKLS

### III. Vaccine/Antigens

There are many vaccines currently available for human and animal use; however, the strategy disclosed herein will work with future vaccines as well.

Vaccine antigens/vaccine derived proteins (which can used alone or in combination) for use in aspects of the invention include, but are not limited to, those antigens found in the following vaccines that immunize against anthrax (AVA (BioThrax); cholera (Vaxchora), COVID-19 (Pfizer-BioNTech; Moderna; Johnson & Johnson's Janssen), diptheria (DTaP (Daptacel, Infanrix); Td (Tenivac, generic); DT (-generic-); Tdap (Adacel, Boostrix); DTaP-IPV (Kinrix, Quadracel); DTaP-HepB-IPV (Pediarix); DTaP-IPV/Hib (Pentacel)), hepatitis A (HepA (Havrix, Vaqta); HepA-HepB (Twinrix)), Hepatitis B (HepB (Engerix-B, Recombivax HB, Heplisay-B); DTaP-HepB-IPV (Pediarix); HepA-HepB (Twinrix)), Haemophilus influenzae type b (Hib) (Hib (ActHIB, PedvaxHIB, Hiberix); DTaP-IPV/Hib (Pentacel)), Human Papillomavirus (HPV) (HPV9 (Gardasil 9) (For scientific papers, the preferred abbreviation is 9vHPV)), Seasonal Influenza (Flu) (IIV* (Afluria, Fluad, Flublok, Flucelvax, FluLaval, Fluarix, Fluvirin, Fluzone, Fluzone High-Dose, Fluzone Intradermal; there are various acronyms for inactivated flu vaccines-IIV3, IIV4, RIV3, RIV4 and ccIIV4; LAIV (FluMist)), Japanese Encephalitis (JE (Ixiaro)), Measles (MMR (M-M-R. II); MMRV (ProQuad)), Meningococcal (MenACWY (Menactra, Menveo); MenB (Bexsero, Trumenba)), Mumps (MMR (M-M-R II); MMRV (ProQuad)), Pertussis (DTaP (Daptacel, Infanrix); Tdap (Adacel, Boostrix); DTaP-IPV (Kinrix, Quadracel); DTaP-HepB-IPV (Pediarix); DTaP-IPV/Hib (Pentacel)), Pneumococcal (PCV13 (Prevnar13); PPSV23 (Pneumovax 23)), Polio (Polio (Ipol); DTaP-IPV (Kinrix, Quadracel); DTaP-HepB-IPV (Pediarix); DTaP-IPV/Hib (Pentacel)), Rabies (Rabies (Imovax Rabies, RabAvert)), Rotavirus (RV1 (Rotarix); RV5 (RotaTeq)), Rubella (MMR (M-M-R II); MMRV (ProQuad)), Shingles (RZV (Shingrix)), Smallpox (Vaccinia (ACAM2000)), Tetanus (DTaP (Daptacel, Infanrix); Td (Tenivac, generic), DT (-generic-), Tdap (Adacel, Boostrix), DTaP-IPV (Kinrix, Quadracel), DTaP-HepB-IPV (Pediarix), DTaP-IPV/Hib (Pentacel)), Typhoid Fever (Typhoid Oral (Vivotif); Typhoid Polysaccharide (Typhim Vi)), Varicella (VAR (Varivax); MMRV (ProQuad)), and/or Yellow Fever (YF (YF-Vax)).

### IV. Cancer Treatment

Immunotherapies have shown great promise but are not effective for all tumor types and are effective in less than 3% of patients with pancreatic ductal adenocarcinomas (PDAC). To make an immune treatment that is effective for more cancer patients and those with PDAC specifically, Salmonella was genetically engineered to deliver antigens directly into the cytoplasm of tumor cells. It was believed that intracellular delivery of an immunization antigen would activate antigen specific CD8 T cells and reduce tumors in immunized mice. To test this hypothesis, intracellular delivering (ID) Salmonella, that deliver a model antigen (ovalbumin) into tumor-bearing, ovalbumin-vaccinated mice, was delivered. ID Salmonella delivers antigens by autonomously lysing in cells after the induction of cell invasion. It was shown that the delivered ovalbumin disperses throughout the cytoplasm of cells in culture and in tumors. This delivery into the cytoplasm is essential for antigen cross-presentation. It was shown that co-culture of ovalbumin recipient cancer cells with ovalbumin specific CD8 T cells triggered a cytotoxic T cell response. After the adoptive transfer of OT-I CD8 T cells, intracellular delivery of ovalbumin reduced tumor growth and eliminated tumors. This effect was dependent on the presence of the ovalbumin-specific T cells. Following an ovalbumin vaccination regimen in mice, intracellular ovalbumin delivery cleared 43% of established KPC pancreatic tumors, increased survival, and prevented tumor re-implantation. This response in the immunosuppressive KPC model demonstrates the potential to treat tumors that do not respond to checkpoint inhibitors, and the response to re-challenge indicates that new immunity was established against intrinsic tumor antigens. In the clinic, ID Salmonella could be used to deliver a protein antigen from a childhood immunization to refocus pre-existing T cell immunity against tumors. As an off-the-shelf immunotherapy, this bacterial system is effective in a broad range of cancer patients.

Bacteria such as Salmonella, Clostridium and Bifidobacterium have a natural tropism for cancers, such as solid tumors. Types of cancer that can be treated using the methods of the invention include, but are not limited to, solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In some aspects, the subject is treated with radiation and chemotherapy before, after or during administration of the bacterial cells described herein.

### V. Administration

The subject can already be vaccinated and thus the subject's immune system recognizes the antigen used in the vaccination, or the subject can first be vaccinated and shortly thereafter the engineered Salmonella can be administered, so as to deliver the antigen to the cancer cells to be recognized/killed by the immune system.

The invention includes administration of the attenuated Salmonella strains described herein and methods for preparing pharmaceutical compositions and administering such as well. Such methods comprise formulating a pharmaceutically acceptable carrier with one or more of the attenuated Salmonella strains described herein.

Delivery of vaccine antigens directly inside into the cytoplasm of cancer cells would refocus vaccine T cells against tumors and be less dependent on inherent tumor characteristics. We have recently created a bacteria-based system that delivers active proteins into tumor cells that could achieve this goal (1). As a result of childhood vaccination, over 90% of individuals have pre-existing immune cells against a number of different pathogens (2). Vaccines generate memory CD8+ T cells that have half-lives of almost 1.5 years in the human body and can be detected for decades (3). Memory CD8+ T cells also rapidly expand and exert persistent cytotoxic responses after engaging with compromised cells (4, 5). Unlike a naïve T cell response, which requires weeks, memory T cells reactivate just days after re-encountering pathogenic antigens presented by infected cells (6, 7). Due to the widespread use of vaccines, the vast majority of the United States population already have endogenous, vaccine-specific, memory T cells that can be readily awakened and redirected against cancer.

For a delivered exogenous antigen to induce a T cell response, it should be available in the cytoplasm to enable immunological presentation and detection [8, 9]. A feature of intracellular Salmonella delivery is that the delivered protein is deposited in the cytoplasm (1). Other intracellular methods deliver proteins to the endosomes, where they are trafficked to the lysosome and degraded (10-12). In contrast, cytoplasmic proteins are processed by the proteasome into small antigenic peptides that are loaded onto major histocompatibility complex-1 (MHC-I) and presented on the cell surface [8, 13-15]. MHC-I loaded peptides from foreign sources elicit a cytotoxic response from activated CD8+ T cells (8, 16). When a T cell recognizes its cognate antigen on MHC-1, it forms a pore into the cancer cell and injects a granzyme cocktail that initiates apoptosis (17-21). Cells cannot avoid programmed cell death once granzymes have been injected (20, 22), which is a critical reason why anti-tumor responses driven by CD8+ T cells are highly effective in treating cancer.

The physiological responses to the presentation of a foreign antigen are steps in the acquisition of antitumor immunity. Cancer cells with genetic mutations typically contain tumor associated antigens (TAAs) that are seen as foreign by the immune system (23-26). However, tumor-derived immune suppression prevents their detection (27). Both T cell activation and cancer cell death promote recognition of TAAs (28, 29). Death of cancer cells releases TAAs into the local environment (30). The TAAs are cross-presented by professional antigen presenting cells (APCs), such as dendritic cells (31-34), to educate memory CD8+ T cells [35-39]. Activated CD8+ T cells secrete Th1 cytokines that induce cross-presentation [40]. The educated memory T cells proceed to kill cancer cell that present their cognate TAAs, a mechanism that is the basis of antitumor immunity (41-44).

Salmonella are particularly well-suited to deliver exogenous vaccine antigens into tumor cells. The intracellular delivery system utilizes bacterial cell invasion to transport proteins into cancer cells (1). After invading into cells, the bacteria express a suicide gene, lysin E, which drives autonomous lysis and releases bacterially expressed proteins (1). In these engineered Salmonella, expression of the regulator gene, flhDC can be used to control the timing and location of cell invasion (1). The use of Salmonella focuses delivery into tumors, because intravenously injected bacteria colonize tumors up to ten thousand-fold more than other organs (46, 47). In addition to these delivery properties, the presence of Salmonella in tumors induces the production of Th1 cytokines, including IFN-γ and IL-2.

Provided herein is an engineered bacterial system that delivers vaccine antigens into tumor cells and show that it harnesses immunity from pre-existing vaccinations to generate a robust antitumor immune response.

A pharmaceutical composition of the invention is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For intravenous administration, suitable carriers include physiological saline, bacteriostatic water, Cremophor EL™ (BASF; Parsippany, N.J.) or phosphate buffered saline (PBS). It must be stable under the conditions of manufacture and storage and must be preserved against the contaminating action of other (undesired) microorganisms. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Injectable solutions can be prepared by incorporating the active compound in the required amount in an appropriate solvent with one or a combination of ingredients discussed above. Generally, dispersions are prepared by incorporating the active compound into a vehicle which contains a basic dispersion medium and various other ingredients discussed above. In the case of powders for the preparation of injectable solutions, the preferred methods of preparation are vacuum drying and freeze-drying which yields a powder of the active ingredient plus any additional desired ingredient from a previously.

Oral compositions generally include an inert diluent or an edible carrier. For example, they can be enclosed in gelatin capsules. For the purpose of oral therapeutic administration, the active compound can be incorporated with excipients and used in the form of tablets, troches, or capsules.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the bacteria are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bacteria are formulated into ointments, salves, gels, or creams as generally known in the art.

It is especially advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

When administered to a patient the attenuated Salmonella can be used alone or may be combined with any physiological carrier. In general, the dosage ranges from about 1.0 c.f.u./kg to about 1×10¹² c.f.u./kg; optionally from about 1.0 c.f.u./kg to about 1×10¹⁰ c.f.u./kg; optionally from about 1.0 c.f.u./kg to about 1×10⁸ c.f.u./kg; optionally from about 1×10² c.f.u./kg to about 1×10⁸ c.f.u./kg; optionally from about 1×10⁴ c.f.u./kg to about 1×10⁸ c.f.u./kg; optionally from about 1×10⁵ c.f.u./kg to about 1×10¹² c.f.u./kg; optionally from about 1×10⁵ c.f.u./kg to about 1×10¹⁰ c.f.u./kg; optionally from about 1×10⁵ c.f.u./kg to about 1×10⁸ c.f.u./kg.

### EXAMPLE

The following example is provided in order to demonstrate and further illustrate certain embodiments and aspects of the present invention and are not to be construed as limiting the scope thereof.

### Example I

### Introduction

Immunotherapy has proven to be extremely effective for many, but not all tumor types (1-3). For pancreatic ductal adenocarcinomas (PDAC), for example, immune checkpoint inhibitors (ICIs) are effective in less than 3% of patients (4-7). Despite the limitation of ICIs, recent successes with chimeric antigen receptor (CAR) T cell therapy in individual patients (8-11), suggests that T cell therapies can be effective against PDAC. Alternate methods are needed to build upon this potential while avoiding the difficulty of scaling these treatments (12). A therapeutic strategy that directs pre-existing pools of T cells against tumors could provide a universal treatment for patients with PDAC and ICI-resistant tumors.

Delivering an antigen from a prior immunization into cancer cells would redirect CD8 T cells from a vaccine against the recipient cells. Delivery into the cytoplasm is a critical component of this technique because it is necessary to induce a cytotoxic T cell response (12, 13). Most protein delivery mechanisms (e.g., nanoparticles, cell-penetrating peptides, and antibody drug conjugates) deliver proteins to early and late endosomes, where they are trafficked to the lysosome and degraded (14-16). In contrast, proteins delivered to the cytoplasm would be processed by the proteasome and antigen-presented on the cell surface (12, 17-19) to interact with CD8 T cells (12, 20). In addition to the direct elimination of presenting cancer cells, recognition of foreign antigens by immune cells in tumors is a critical step that can lead to the acquisition of antitumor immunity (21-24).

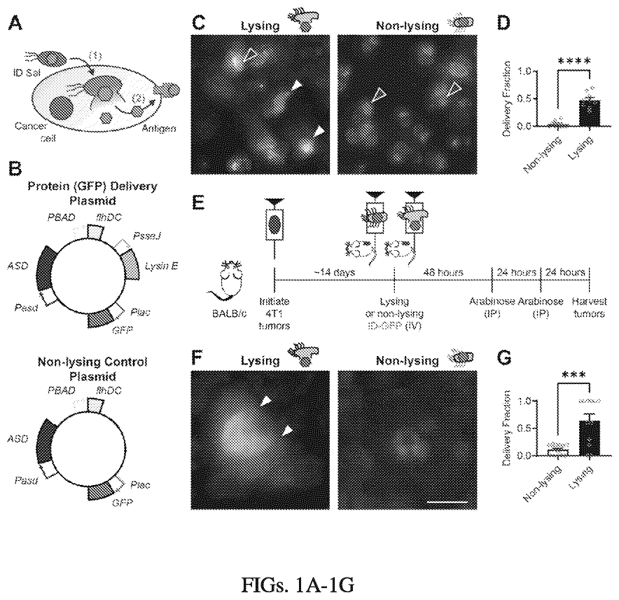

An intracellular delivering (ID) Salmonella was created to release proteins into the cytoplasm of cancer cells (FIG. 1A) (25). This delivery system utilizes innate Salmonella mechanisms (26, 27) to control invasion into cancer cells (25). After cell invasion, an engineered gene circuit triggers bacterial lysis and releases expressed proteins (25). The autonomous lysis system makes the therapy safe and non-toxic by clearing the bacteria after delivery of the protein payload (25). In addition to cytoplasmic delivery, ID Salmonella accumulate in tumors over healthy organs more than 3000-fold after intravenous injection (28, 29). There are five predominant mechanisms that lead to this accumulation: (1) increased blood flow following inflammation (41); (2) entrapment in the tumor vasculature (28); (3) chemotaxis into the tumor interstitium (42, 43); (4) preferential replication in the tumor microenvironment (42, 43); and (5) immune protection in the privileged tumor microenvironment (44). Other strategies have demonstrated the potential of microbial immunotherapies by showing that engineered bacteria can deliver tumor neoantigens (30) and checkpoint nanobodies (31) into tumors, while promoting T cell infiltration (32).

Herein the adaption of ID Salmonella to deliver immunization antigens into cancer cells is described. It is believed that delivering an exogenous antigen with this system activates antigen specific CD8 T cells, reduces tumor volume, and increases survival in immunized mice. To test this, ID Salmonella was engineered to deliver ovalbumin as a model of an antigen from a prior immunization. We used an in vitro cell invasion assay, T cell co-culture, and fixed-cell microscopy to quantify delivery into cancer cells and measure the CD8 T cell response. Adoptive T cell transfer and immunization were used to quantify the effect of intracellular antigen delivery on tumor growth and survival. We re-challenged mice with cleared tumors to explore the extent that this treatment forms antitumor immunity. These immune responses were measured in the highly immunosuppressive KPC tumor model that does not respond to ICIs (45, 46). Results from these experiments show that by refocusing pre-existing, T cell immunity against tumors, antigen delivery with ID Salmonella is an immunotherapy that could be effective for a wide range of cancer patients.

### Materials and Methods

Delivering a model vaccine antigen into tumor cells with Salmonella activates antigen specific CD8+ T cells, reduces tumor volume, and increases survival in mice that were previously immunized against the antigen. This was demonstrated by creating Salmonella that autonomously lysis after cell invasion and deliver ovalbumin into the cytosol of cancer cells. Ovalbumin-specific OT-1 T cells were used to show that bacterial delivery could induce antigen specific toxicity to cancer cells in vitro. We adoptively transferred, OT-I T cells into tumor bearing mice to demonstrate that bacterially delivered ovalbumin could induce an antitumor immune response. Bacteria were administered into ovalbumin-vaccinated, tumor-bearing mice to demonstrate redirection of vaccine immunity in a more clinically relevant model. After complete clearance of some primary tumors, mice were re-challenged with cancer cells to demonstrate the acquisition of antitumor immunity. Delivering vaccine antigen selectively into tumors with engineered Salmonella would enable treatment in a broader group of cancer patients regardless of an individual's tumor mutational status. Moreover, a single strain can be used to deliver the same vaccine antigen into many patients provided that the associated vaccine has been widely administered across the population. Vaccine antigen delivery with Salmonella has the potential to be a highly effective, off-the shelf immunotherapy that produces durable antitumor immune responses in a broad range of cancer patients.

### Plasmid Design and Strains

The protein delivery plasmid contains four gene circuits that activate intracellular lysis (PsseJ-LysE), control invasion (PBAD-flhDC), express GFP (Plac-GFP-myc), and maintain copy number (Pasd-ASD). The non-lysing control plasmid does not contain the intracellular lysing (PsseJ-LysE) circuit. The myc tag was added to the GFP to facilitate detection. Both of these plasmids contain the ColE1 origin and ampicillin resistance, and their creation is described previously (33). To create the ovalbumin delivery plasmid, the ova gene was amplified from #64599 using plasmid (Addgene) primers CCGCATAGTTAAGCCAGTATACATTTACACTTTATGCTTCCGGCTCGTATAATAA AAAAAAAAAAAAGGAGGAAAAAAAATGGGCTCCATCGGTGCAG (SEQ ID NO 100) and CTACAGATCCTCTTCTGAGATGAGTTTTTGTTCAGGGGAAACACATCTGCCAAA (SEQ ID NO: 101). The delivery plasmid was amplified using primers TCATCTCAGAAGAGGATCTGTAACTCCGCTATCGCTACGTGA (SEQ ID NO: 102) and TGTATACTGGCTTAACTATGCGG (SEQ ID NO: 103). This PCR amplification preserved all genes within the plasmid and exchanged the Plac-GFP-mye genetic circuit for Plac-ova-myc. These plasmids were transformed into the AflhD, Aasd strain of VNP20009 as described previously (33) to generate ID-GFP and ID-OVA Salmonella. To detect antigen expression, ID-OVA was suspended in Laemmli buffer and myc-tagged ovalbumin was identified by immunoblot with rat anti-myc antibody (Chromotek).

### Cell Culture

Four cancer cell lines were used in this study: 4T1 murine breast carcinoma cells, MC38 murine colon cancer cells, Hepa 1-6 murine hepatocellular carcinoma cells, and KPC PDA murine pancreatic cancer cells (ATCC, Manassas, VA). KPC (LSL-Kras^{G12D/+}; LSL-Trp53^{R/72H/+}; Pdx-1-Cre) PDA and 4T1 cells were grown and maintained in Dulbecco's Minimal Eagle Medium (DMEM) containing 3.7 g/L sodium bicarbonate and 10% fetal bovine serum. MC38 cancer cells were grown in RPMI-1640 supplemented with 2 g/L sodium bicarbonate, 10% fetal bovine serum and penicillin/streptomycin. For microscopy studies, 4T1 cancer cells were incubated in DMEM with 20 mM HEPES buffering agent and 10% FBS.

### Microscopy

Samples were imaged on a Zeiss Axio Observer Z.1 microscope. Fixed cells on coverslips were imaged with a 100× oil immersion objective (1.4 NA). Tumor sections were imaged with 20× objectives (0.3 and 0.4 NA, respectively). Fluorescence images were acquired with either 480/525 or 525/590 excitation/emission filters. All images were background subtracted and contrast was uniformly enhanced.

### Immunocytochemistry to Detect Protein Delivery in Cancer Cells

To visualize and measure protein delivery. ID Salmonella were administered to cancer cells grown on glass coverslips. To prepare the coverslips, they were placed in 12-well plates and sterilized with UV light in a biosafety hood for 20 minutes. Cancer cells (either 4T1 or Hepa 1-6 cells) were seeded on the coverslips at 40% confluency and incubated overnight in DMEM. Concurrently, Salmonella were grown to an optical density (OD; at 600 nm) of 0.8. After incubation, the Salmonella were added to the cancer cell cultures and allowed to infect the cells for two hours. After this invasion period, the cultures were washed five times with 1 ml of phosphate buffered saline (PBS) and resuspended in 2 ml of DMEM with 20 mM HEPES, 10% FBS and 50 μg/ml gentamycin. The added gentamycin removes extracellular bacteria. After twenty-four hours of incubation, the media was removed and the coverslips were fixed with 10% formalin in PBS for 10 minutes. After fixing, the coverslips were blocked with intracellular staining buffer (ISB; phosphate-buffered saline [PBS] with 0.1% Tween 20, 1 mM EDTA, and 2% bovine serum albumin [BSA]) for 30 minutes. The Tween 20 in this buffer selectively permeabilizes mammalian cell membranes, while leaving bacterial membranes intact, as previously described (33). After permeabilization, coverslips were stained to identify Salmonella and delivered protein. Stained coverslips were washed three times with ISB and mounted to glass slides using 20 μl mountant with DAPI (ProLong Gold Antifade Mountant, ThermoFisher). Mounted coverslips were cured overnight at room temperature. Coverslips were imaged as described in the microscopy section.

### Measurement of Delivery Fraction

ID Salmonella was administered to cancer cells to measure the fraction of cells with delivered protein. Two experiments were used to measure (1) the necessity of the lysis gene circuit, and (2) the efficacy of delivering ovalbumin. The necessity of the PsseJ-LysE was measured by growing ID-GFP and non-lysing ID-GFP to an OD of 0.8 and infecting 4T1 cells at a multiplicity of infection (MOI) of 10 for two hours. The delivery of ovalbumin was measured by growing ID-OVA and ID-GFP to an OD of 0.8 and infecting Hepa 1-6 cells at an MOI of 20 for two hours. For both experiments, the bacteria were induced with 20 mM arabinose during co-infection. To eliminate extracellular bacteria after infection, the cells were washed five times with PBS and fresh media containing 50 μg/ml of gentamycin was added. After 24 hours of incubation, the coverslips were fixed and incubated in ISB for 30 minutes. Cells were stained to identify Salmonella with FITC anti-Salmonella antibody (Abcam; 1:200 dilution) and GFP-myc, or OVA-myc with an anti-myc antibody (9E1, Chromotek; 1:200 dilution) for one hour at room temperature in a humidified chamber. Coverslips were incubated with secondary antibody (anti-rat alexa-568 antibody; 1:200 dilution) for one hour at room temperature.

Delivery fraction was quantified on a per-cell basis by assessing if cells were invaded with bacteria and contained delivered protein. Invaded cells were identified as nuclei bordering intracellular Salmonella. Cells with delivered protein stained for GFP throughout the cytosol. Delivery fraction was the number of cells with cytosolic protein delivery divided by the total number of infected cells. Image analysis was blinded and conducted without knowledge of the treatment group.

### Imaging Ovalbumin Delivery

Detailed images of delivered ovalbumin were obtained using the immunocytochemistry technique described above. ID-OVA was grown to an optical density of 0.8 and added to cultures of 4T1 cells at a multiplicity of infection (MOI) of 10 for two hours. After infection, the cells were washed, and 50 μg/ml of gentamycin was added. After 24 hours of incubation, the coverslips were fixed and stained to identify OVA-myc with anti-myc antibody (9E1, Chromotek; 1:200 dilution). After primary staining, coverslips were incubated with secondary antibody (anti-rat alexa-488 antibody; 1:200 dilution) and Alexaflor-568-conjugated phalloidin (ThermoFisher; 1:200 dilution) to identify f-actin.

### Immunohistochemical Detection of GFP Delivery In Vivo

To identify and quantify GFP delivery to tumor cells, two groups BALB/c mice with 4T1 tumors were injected with 2×106 CFU of either ID-GFP or non-lysing ID-GFP Salmonella. Both groups of mice were injected (IP) with arabinose at 48 and 72 h post bacterial injection to induce flhDC expression. Ninety-six hours after bacterial injection, mice were sacrificed, and tumors were excised.

Tumor sections were fixed in 10% formalin for 3 days. Fixed tumor samples were stored in 70% ethanol for 1 week. Tumor samples were embedded in paraffin and sectioned into 5 μm sections. Deparaffinization was performed by washing the sectioned tissue three times in 100% xylene, twice in 100% ethanol, once in 95% ethanol, once in 70% ethanol, once in 50% ethanol, and once in DI water. Each wash step was performed for 5 minutes. Antigen retrieval was performed by incubating the tissue sections in 95° C., 20 mM sodium citrate (pH 7.6) buffer for 20 minutes. Samples were left in sodium citrate buffer until the temperature reduced to 40° C. Samples were then rehydrated with two quick (<1 minute) rinses in DI water followed by one five-minute wash in TBS-T.

Prior to staining, tissue sections were blocked with Dako blocking buffer (Dako) for one hour. Tissue sections were stained to identify Salmonella and released GFP with 1:100 dilutions of [1] FITC-conjugated rabbit anti-Salmonella polyclonal antibody (Abcam, catalog #ab69253), and [2] rat anti-myc monoclonal antibody (Chromotek) in Tris buffered saline with 0.1% Tween 20 (TBS-T) with 2% BSA (FisherScientific). Sections were washed three times in TBS-T w/2% BSA and incubated with Alexaflor-568 goat anti-rat secondary antibodies (ThermoFisher). After washing sections three times with TBS-T, 40 μl of mountant with DAPI (ThermoFisher) and a cover slip were added to each slide. Slides were incubated at room temperature for 24 hours until the mountant solidified. Slides were imaged as described in the microscopy section.

Delivery fraction in tumor sections was quantified using a similar method as with fixed cells on cover slips described above. Invaded cells were identified as nuclei bordering intracellular Salmonella and cells with delivered protein had GFP throughout the cytosol. The delivery fraction was the number of cells with delivered protein divided by the total number of infected cells. Image analysis was blinded and conducted without knowledge of the treatment group.

### CD8 T Cell Activation and Culturing

To isolate OT-I CD8 T cells, the spleen and inguinal lymph nodes were harvested from female OT-I mice. The lymphoid tissue was mechanically dissociated in PBS using the end of a syringe. A single cell suspension was produced by passing the organ slurry through a 40-micrometer cell strainer. Naïve OT-I T cells were purified using a negative selection kit (Biolegend). This negative selection purified approximately eight to ten million naïve OT-IT cells, which were 91% pure.

The isolated T cells were activated using anti-CD3 and anti-CD28 antibodies and either (1) a plate-bound method or (2) magnetic beads (Thermo-Fisher). To prepare the antibody plate, anti-CD38 antibody (Biolegend) was added in 2 ml of PBS to a T25 flask at a concentration of 4 μg/ml and incubated at 37° C. overnight. The flask was washed twice with 5 ml of PBS to remove unbound antibody. For both methods, one million purified, naïve OT-IT cells were added to 5 ml of complete RPMI media (2 mM glutamine, 2 mM sodium pyruvate, 20 IU/ml recombinant mouse IL-2, 50 μM beta-mercaptoethanol and 12.5 μg/ml amphotericin B in RPMI media). For the plate bound method, the T cells were added to treated flask and the medium was supplemented with 2 μg/ml of anti-CD28 antibody (Biolegend). For the bead method, 25 μl of washed CD3/CD28 Dynabeads were added to naïve T cells. After incubating at 37° C.′ for 96 hours, cell clusters were gently broken apart by pipetting. A magnet was used to separate the magnetic beads from the activated T cells. The separated T cells were washed twice with PBS, re-suspended in complete RPMI medium and maintained at a concentration of 1 million cells/ml.

Five days after starting the activation process, the OT-I T cells were stained against CD8 and CD44 to assess purity and extent of activation, respectively. The anti-CD8 and anti-CD44 antibodies were conjugated to APC and FITC (Biolegend), respectively, and diluted 1:500 in extracellular staining buffer (ESB; PBS with 1 mM EDTA and 2% BSA). Stained samples were evaluated on a Novocyte flow cytometer. Fluorescence minus one and unstained T cells were used as gating controls.

T Cell Cytotoxicity after Ovalbumin Delivery In Vitro

To measure the effect of bacterial ovalbumin delivery on T cell-cytotoxicity, OT-I T cells were applied to cancer cells after being infected with antigen-delivering Salmonella. ID-GFP and ID-OVA were grown to an OD of 0.8 in LB. These bacteria were added to well-plates containing 60% confluent Hepa 1-6 cells at an MOI of 20 for two hours. The bacteria were induced with 20 mM arabinose during the 2-hour infection. After infection, the cancer cells were washed five times with PBS to eliminate extracellular bacteria. The cells were incubated in complete RPMI medium containing 50 μg/ml gentamycin and 1 μM calcein-AM for 30 minutes. The cells were washed three times with PBS to eliminate the extracellular calcein-AM. These treated Hepa 1-6 cells were incubated with isolated and activated OT-I CD8 T cells at an effector-to-target ratio of 10:1 complete RPMI medium (50 μM beta-mercaptoethanol, 20 IU IL-2/ml, 2 mM sodium pyruvate, and 2 mM glutamine) for 48 hours. At the end of the incubation period, 200 μl of RPMI media was sampled from each of the wells. The 200 μl samples was centrifuged at 1000×g for 5 minutes. For each 100 μl sample, the fluorescence intensity from released calcein was quantified using a plate reader (Biotek).

Efficacy of Ovalbumin Delivery in Mice after T Cell Adoptive Transfer

Two groups of six week-old C57BL/6 mice were subcutaneously injected with 1×105 MC38 cancer cells. Once tumors reached approximately 50 mm3, the mice were intratumorally injected with 4×10⁷ GFP-delivering (ID-GFP) or ovalbumin-delivering (ID-OVA) Salmonella. Forty-eight hours days after bacterial injection, one million activated, OT-I T cells were adoptively transferred into each mouse through the tail vein. In addition, 48 and 72 hours after bacterial injection, the mice were injected (IP) with 100 mg of arabinose in 400 μl of PBS to induce flhDC expression. The bacteria and T cell administration cycle was performed twice for each mouse. Tumor volumes were measured with a caliper twice a week until they reached maximum volume limits or cleared. Tumor volumes were calculated using the formula (Length)×(Width)²/2.

The effect of ovalbumin delivery in the absence of adoptive transfer was measured in two groups of female mice that were subcutaneously injected with 1×10⁵ MC38 cells. Once tumors were approximately 50 mm3, mice were intratumorally injected with 4×10⁶ CFU of ID-GFP or ID-OVA every four days. One hundred milligrams of arabinose were injected IP into the mice at 48 and 72 hours after bacterial injection. Tumors were measured with calipers every 3 days until mice reached maximal tumor burden.

Delivery and Efficacy of Ovalbumin Delivery In Vivo after Immunization

Two groups of six-week-old female C57BL/6 mice were immunized by two IP injections of 100 μg ovalbumin and 100 μg poly(I:C) in 100 μl PBS spaced seven days apart. Fourteen days after the immunization booster, the mice were subcutaneously injected with 1×10⁵ MC38 cancer cells on the hind flank. Once the tumors reached approximately 50 mm3, the mice were intratumorally injected with 4×10⁷ of either GFP-delivering (ID-GFP) or ovalbumin-delivering (ID-OVA) Salmonella. Forty-eight hours after bacterial injection, the mice were injected (IP) with 50 μg of anti-PD-1 checkpoint blockade antibodies (Biolegend). In addition, 48 and 72 hours after bacterial injection, mice were injected IP with 100 μg arabinose. The treatment cycle was performed twice for each mouse. Tumor volumes were measured with calipers twice a week until they reached maximum volume limits. Tumor volumes were calculated using the formula (Length)×(Width)²/2.

Treatment of Immunized Mice with ID-OVA and Tumor Re-Challenge

Four groups of female C57BL/6 mice were immunized with 100 μg ovalbumin and 50 μg poly(I:C) in 100 μl PBS by IP injection, 28 days apart. One week after the second immunization, the mice were subcutaneously injected with 2×10⁵ KPC PDAC cells (Kerafast) on the right flank. Once tumors reached approximately 30-50 mm3, the mice were injected intratumorally with either 1×10⁷ CFU of ID-OVA, 1×10⁷ CFU of ID-GFP (bacterial control), saline, or intraperitoneally injected with 50 mg/kg gemcitabine every 5 days. All mice were injected (IP) with 400 mg of arabinose 48 and 72 hours after therapeutic administration. Tumors were measured using calipers every three days. Tumor volumes were calculated using the formula (length*width²)/2. Mice that completely cleared tumors were re-challenged on the left flank 14 days after primary tumor clearance and monitored for tumor regrowth for a minimum of 14 days.

### Results

Engineered Salmonella Deliver Exogenous Antigens into Cancer Cells

Intracellular delivering (ID) Salmonella were created by transformation with a delivery platform that controls cell invasion, triggers intracellular lysis and delivers proteins into cancer cells (FIG. 1B, top). This plasmid contains genetic circuits that (1) constitutively produce green fluorescent protein (GFP), Plac-GFP; (2) control cell invasion, PBAD-flhDC; (3) maintain plasmids after injection in mice, Pasd-asd; and (4) lyse the bacteria after cell invasion, PsseJ-LysE. A control strain was created by transforming bacteria with a plasmid that produces GFP (Plac-GFP) and controls invasion (PBAD-fhDC) but does not contain the genetic circuit for autonomous lysis (PsseJ-LysE; FIG. 1B, bottom). When administered to 4T1 cancer cells, ID Salmonella delivered GFP into the cellular cytoplasm (FIG. 1C, left). Non-lysing controls did not release any GFP (FIG. 1C, right). Lysing Salmonella delivered GFP to significantly more cells than non-lysing controls (P<0.0001; FIG. 1D).

To measure the extent that the lysis system promotes protein delivery to cancer cells in tumors, ID-GFP Salmonella were administered to mice with 4T1 mammary tumors (FIG. 1B). Control mice were administered ID Salmonella that do not lyse. Two days after bacterial injection, all mice were injected with arabinose to activate the PBAD-flhDC circuit and induce cell invasion (FIG. 1E). In mice that received ID-GFP Salmonella, the cytosol of cancer cells was filled with bacterially produced GFP (FIG. 1F, left). In control mice, cells contained Salmonella, but these intracellular bacteria did not release any GFP. (FIG. 1F, right). ID-GFP Salmonella delivered protein to significantly more cells than control bacteria (P=0.0001, FIG. 1G). In cells with intracellular bacteria, ID-GFP Salmonella delivered GFP to more than 60% of cells (P=0.0002, FIG. 1G).

### Intracellular Bacterial Antigen Delivery Induced a Cytotoxic CD8+ T Cell Response

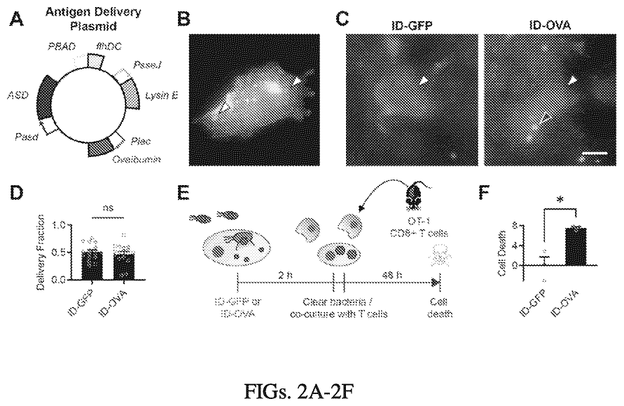

To create the bacterial immunotherapy, we transformed Salmonella with a plasmid that encodes for the production and intracellular release of ovalbumin, as a model of an immunization antigen (FIG. 2A). This engineered ID-OVA strain has the same circuits as ID-GFP to control invasion and lysis. When administered to 4T1 cancer cells, ID-OVA lysed and delivered ovalbumin that diffused throughout the cytosol (FIG. 2B). Administration of either ID-GFP or ID-OVA equally delivered proteins into approximately 50% of cells (FIG. 2C-D).

To measure the effect of ovalbumin delivery on T cell cytotoxicity, ID-OVA Salmonella were administered to Hepa 1-6 cancer cells for 2 hours (FIG. 2E). The response was compared to administration of ID-GFP as a control. After removal of extracellular bacteria, activated OT-I CD8 T cells were immediately added to the cultures for 48 hours at a ratio of ten CD8 T cells to one cancer cell. In these co-cultures, the CD8 T cells killed more cancer cells after administration of ID-OVA compared to control ID-GFP Salmonella (P<0.05, FIG. 2F).

### Exogenous Antigen Delivery to Tumors Induced an Antigen-Specific T Cell Response

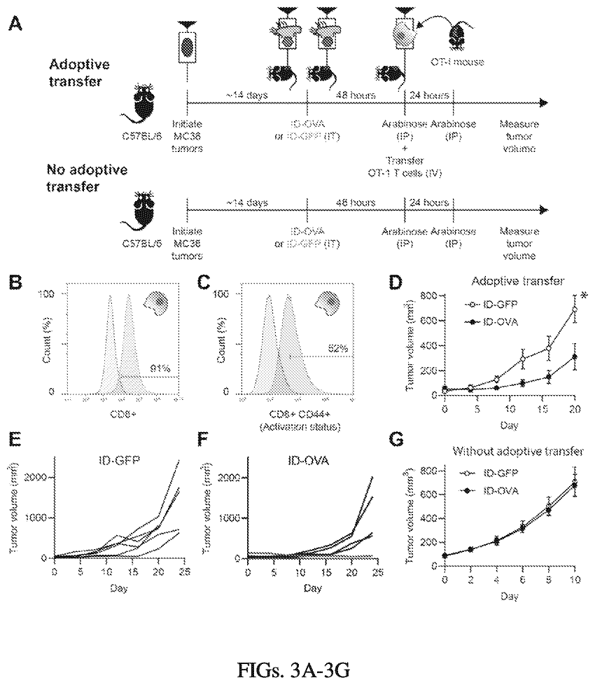

To test if exogenous protein delivery could induce an antigen-specific T cell response, ID-OVA Salmonella were administered to mice with MC38 tumors (FIG. 3A). Five days after intratumoral injection of either ID-OVA or control ID-GFP, half of the mice were injected with activated, ovalbumin-specific CD8 T cells (FIG. 3A). No T cells were transferred into the remaining mice (FIG. 3A). The injected OT-I T cells were 91% pure and over half expressed high levels of the activation marker, CD44 (FIG. 3C). Mice treated with ID-OVA had significantly reduced tumor growth compared with mice treated with ID-GFP controls (P <0.05; FIG. 3D). None of the six mice treated with ID-GFP responded to bacterial injection (FIG. 3E). In the ID OVA group, one mouse had a partial response, and another had a complete response (red lines, FIG. 3F). In the groups without adoptive transfer, there was no difference in tumor response between mice that received ID-OVA and ID-GFP (FIG. 3G), indicating that the tumor response was mediated by the OT-I CD8 T cells.

Refocus of Vaccine Immunity Against Tumors with Bacterial Antigen Delivery

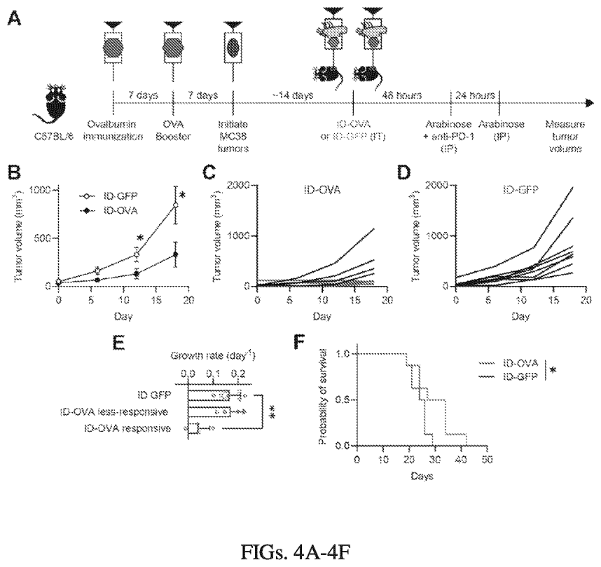

To test whether pre-existing, vaccine-generated immunity could be retargeted against cancer, antigen-delivering ID Salmonella were administered to vaccinated, tumor-bearing mice (FIG. 4A). To establish immunity to an exogenous antigen, mice were vaccinated with two doses of ovalbumin and poly(I:C), which is a Th1 adjuvant that activates CD8 T cells (FIG. 4A). One week after the second vaccine dose, MC38 tumors were implanted in the mice. When the tumors formed, the mice were intratumorally injected with 2×107 CFU (colony forming units) of either ID OVA or control ID-GFP (FIG. 4A). Tumor growth in mice injected with ID-OVA Salmonella was significantly reduced compared to mice injected with control ID-GFP (P<0.05; FIG. 4B). Four of the eight mice injected with ID-OVA had no significant tumor growth over eighteen days of observation (FIG. 4C). In comparison, all tumors grew in control ID-GFP mice over the same period (FIG. 4D). The growth rate of responsive ID-OVA tumors was 25% of ID-GFP tumors (P=0.0012, FIG. 4E). Mice administered with ID-OVA bad prolonged survival compared to mice injected with ID-GFP (P=0.0480, FIG. 4B). Bacterial delivery of a vaccine antigen cleared pancreatic tumors and prevented tumor re-challenge

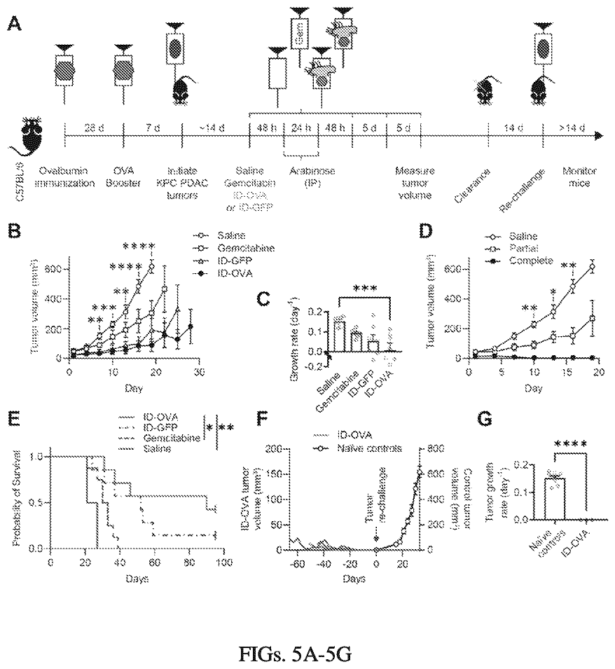

To test its efficacy against pancreatic cancer, ID-OVA was administered to immunocompetent C57BL/6 mice with KPC tumors (FIG. 5). The KPC tumor model is driven by KRAS and p53 mutations that are common in human pancreatic cancer (45). The tumors have highly immunosuppressive microenvironments and do not respond to ICIs (45, 46). Four groups of mice were immunized with of two doses of ovalbumin and poly(I:C) and implanted with KPC pancreatic ductal adenocarcinoma (PDAC) tumors on the flank (FIG. 5A). These mice were injected with one of four treatments (1) saline, (2) gemcitabine, (3) control ID-GFP Salmonella, or (4) ID-OVA Salmonella. Gemcitabine is a standard therapy for pancreatic cancer. Tumor clearance was monitored for 14 days, after which some mice were re-challenged with KPC PDAC cells on the opposite flank (FIG. 5A). ID-OVA significantly reduced tumor volume compared to saline controls (FIG. 5B). On day 19, the average tumor in ID-OVA-treated mice was 14% of saline treated mice (P<0.0001). Treatment with ID-OVA significantly reduced the growth rate of KPC PDAC tumors (P=0.0004, FIG. 5C). Of the mice treated with ID-OVA, three had a complete response and four had partial responses (FIG. 5D). Between days 10 and 16, the average tumor size in mice with partial responses to ID-OVA was 49% of saline-treated controls (P=0.0046 on d 16; FIG. 5D).

Treatment with ID-OVA antigen-delivering bacteria increased mouse survival and prevented tumor re-implantation (FIG. 5E-G). In these mice with KPC PDAC tumors, ID-OVA significantly increased survival compared to both saline (P=0.0012) and gemcitabine (P =0.026). The median survival after treatment with ID-OVA was 90 days compared to 31.5 and 52 days for gemcitabine and ID-GFP. In three of the treated mice, ID-OVA eliminated tumors by days 31, 46 and 52 (FIG. 5F). Two weeks after tumor clearance, these three mice were re-challenged with KPC PDAC cells in the opposite flank and monitored for at least four weeks. No tumors formed in any of the mice (FIG. 5F). For comparison, naïve tumors grew at a rate of 0.14 d-1 (P<0.0001, FIG. 5G). These results show that bacterial delivery of an immunization antigen induces a durable response that prevents the establishment of new tumors.

### CONCLUSIONS

Engineered Salmonella delivered vaccine associated protein into the cytosol of cancer cells, which, is a step in MHC-I dependent antigen presentation. Delivery of the model protein, ovalbumin, into cancer cells in vivo generated an anti-tumor immune response from adoptively transferred, ovalbumin specific CD8 T cells. Tumor bearing mice that were vaccinated against ovalbumin exhibited slower tumor growth and prolonged survival when administered with ovalbumin delivering Salmonella. This is the first study to demonstrate that bacteria can be used as an off-the-shelf approach to repurpose vaccine related immune cells to target tumor cells. The bacterial vaccine protein delivery technology described is rapidly scalable and has broad applicability to cancer patients with preexisting immunity to other pathogen associated proteins generated either from infection or vaccination.

### Discussion

These results show that intracellular delivery of an immunization antigen with engineered Salmonella induces T cell cytotoxicity and eliminates tumors. When Salmonella delivered exogenous antigens into the cytoplasm of cancer cells in tumors, the peptides dispersed throughout the cytoplasm (FIGS. 1 & 2). Bacterial delivery of ovalbumin marked cancer cells as immunological targets to be cleared by CD8 T cells (FIG. 2). In mice, intracellular delivery of ovalbumin reduced the volume of colon and pancreatic tumors (FIGS. 3-5). The dependence on adoptive transfer suggests that the tumor response was mediated by the CD8 T cells (FIG. 3). In tumors, the induced T cell-cytotoxicity (FIGS. 3-5) matched the cytotoxicity observed in culture (FIG. 2). Bacterial delivery of ovalbumin to immunized mice reduced tumor volume and increased survival (FIG. 4), suggesting that intracellular antigen delivery redirects vaccine immunity to tumors. Coupling vaccination with intracellular antigen delivery eliminated pancreatic tumors and prevented tumor re-implantation (FIG. 5). Efficacy in the immunosuppressive KPC model demonstrates the clinical potential of the approach to overcome immune resistance in PDAC.

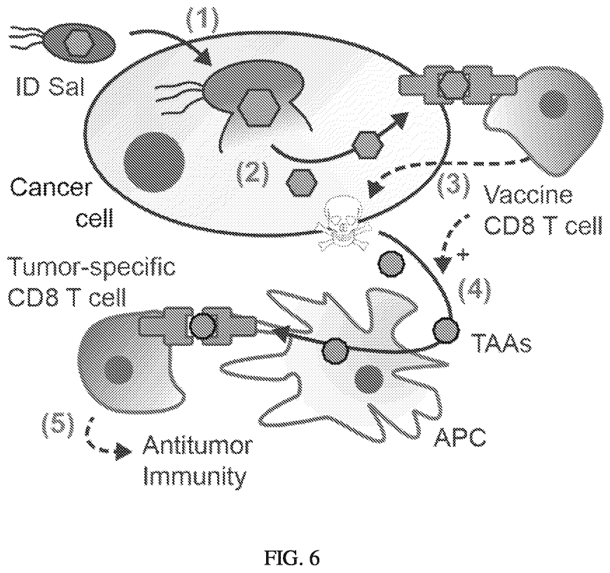

The prevention of tumor re-challenge suggests that bacterial antigen delivery triggers the formation of antitumor immunity (FIG. 6). In this mechanism, recognition of the vaccine antigen on the surface of cancer cell initiates an antigen cascade that leads to the formation of immunity against intrinsic tumor antigens (21-24). When co-cultured with cancer cells, ovalbumin-specific OT-I CD8 T cells preferentially killed cancer cells with bacterially delivered ovalbumin (FIG. 2F). This specificity suggests that T cells recognized the ovalbumin antigen presented on the surface of the cancer cells (steps 1-3 in FIG. 6). In mice, the dependence on transferred CD8 T cells (FIG. 3G) indicates that T cell-mediated cytotoxicity is an essential component of the tumor response. In vaccinated mice, the tumor response was greater when the delivered antigen matched the vaccine antigen (FIG. 4), suggesting that the vaccine T cells specifically recognized the delivered antigen. The development of the antitumor immunity (FIG. 5) suggests that CD8 T cells played a critical role in the tumor response (47). The resistance to re-implantation of tumor cells, which did not contain ovalbumin (FIG. 5), suggests that the developed immunity was to intrinsic tumor antigens (steps 4-5 in FIG. 6).

The delivery of immunogenic antigens to tumors with Salmonella most likely induced a CD4 T cell response. Many groups have shown that Salmonella colonization in tumors activates CD4 T cells and induces the production of Tₕ1 cytokines (30, 48-51). Infiltration of CD4 T cells is required for activation of CD8 T cells (52-54) and the tumor responses seen here (FIGS. 3-5). The Tₕ1 cytokines produced by CD4 cells induce antigen-presenting cells (APCs) to cross-present tumor associated antigens (55-58) and are critical factors in the acquisition of antitumor immunity (FIG. 6).

Immunization with the antigen prior to bacterial delivery is necessary because of the time required to form immunity. It is possible that OVA presentation after Salmonella delivery could have formed memory CD8 T cells (59). However, we did not see a tumor response after administering ID-OVA Salmonella to non-immunized mice that did not receive adoptively transferred CD8 T cells (FIG. 3G). A likely reason for this lack of response is the time required (typically 4-8 days) to form memory CD8 T cells to a novel antigen (60). In addition, the memory CD8 T cell response could have been stronger after immunization because of Th1 adjuvant in the vaccine.

In the clinic, Salmonella-based antigen delivery could provide comprehensive, off-the-shelf immunotherapy. By utilizing established immunity to vaccine proteins, specific tumor antigens would not need to be identified, and the therapy could be effective against many tumors without modification. Rather than a model antigen, this bacterial system could deliver a protein antigen from a childhood vaccine to refocus the pre-existing vaccine immunity towards tumors. A single bacterial strain could be used for many patients, as long as the associated vaccine was widely administered across the population. Most (90.8%) adults in the United States have received immunizations that form memory CD8 T cells against multiple viral antigens (25-27). Without the need for tumor-specific antigenic profiling, antigen-delivering bacteria could prevent the formation of new tumors and metastases, similar to the re-challenge response observed in mice (FIG. 5).

To make this strategy broadly effective in the clinic, it could be used with multiple vaccine antigens. This is possible because of the large genetic capacity of engineered bacteria to express multiple recombinant proteins. The average person has been administered nine different vaccines by three years of age (61). Engineered Salmonella could be designed to deliver a combinatorial range of vaccine-derived proteins to take advantage of this breadth of intrinsic immunity. Delivering multiple antigens would increase the probability that vaccine-associated T cells would infiltrate and activate within tumor tissue. An additional strategy that would increase efficacy would be delivery of booster vaccines to patients prior to bacterial antigen delivery. An antigen-specific booster would increase the number of vaccine-specific T cells in circulation and, therefore, the likelihood that vaccine T cells efficiently destroy cancer cells that present the exogenous vaccine antigen.

This study is the first to demonstrate that Salmonella can be used to repurpose immunization derived immune cells to target tumors. A bacterial approach could provide new therapeutic options for patients with late-stage pancreatic cancer or patients with immunosuppressive tumors that do not respond to checkpoint inhibitors. It would be widely applicable to most patients with pre-existing immunity to vaccine antigens and would be less dependent on tumor subtype. Because the engineered Salmonella only lyse inside cells in tumors (25), the delivered antigen would be shielded from immunological detection and premature clearance in the blood. This therapy would be particularly beneficial if it increased recognition of tumor antigens and formed antitumor immunity, as suggested by the tumor re-challenge results. Redirecting pre-existing immune cells to fight cancer with tumor-selective Salmonella could serve as a rapidly deployable therapy that would be effective for many patients.

### BIBLIOGRAPHY

1. Torphy R J, Zhu Y W, Schulick R D. Immunotherapy for Pancreatic Cancer: Barriers and Breakthroughs. Ann Gastroent Surg (2018) 2(4):274-81. doi: 10.1002/ags3.12176.
2. Henriksen A, Dyhl-Polk A, Chen I, Nielsen D. Checkpoint Inhibitors in Pancreatic Cancer. Cancer treatment reviews (2019) 78:17-30. doi: 10.1016/j.ctrv.2019.06.005.
3. Haslam A, Prasad V. Estimation of the Percentage of Us Patients with Cancer Who Are Eligible for and Respond to Checkpoint Inhibitor Immunotherapy Drugs. JAMA Network Open (2019) 2(5): e192535. doi: 10.1001/jamanetworkopen.2019.2535.
4. Pretta A, Lai E, Persano M, Donisi C, Pinna G, Cimbro B, et al. Uncovering Key Targets of Success for Immunotherapy in Pancreatic Cancer. Expert Opin Ther Targets (2021) 25(11):987-1005. Epub 2021 Nov. 23. doi: 10.1080/14728222.2021.2010044.
5. Skelton R A, Javed A, Zheng L, He J. Overcoming the Resistance of Pancreatic Cancer to Immune Checkpoint Inhibitors. J Surg Oncol (2017) 116(1):55-62. doi: 10.1002/jso.24642.
6. Wandmacher A M, Letsch A, Sebens S. Challenges and Future Perspectives of Immunotherapy in Pancreatic Cancer. Cancers (2021) 13(16):18. doi: 10.3390/cancers13164235.
7. Kabacaoglu D, Ciecielski K J, Ruess D A, Algül H. Immune Checkpoint Inhibition for Pancreatic Ductal Adenocarcinoma: Current Limitations and Future Options. Front Immunol (2018) 9:1878. Epub 2018 Aug. 31. doi: 10.3389/fimmu.2018.01878.
8. Leidner R, Sanjuan Silva N, Huang H, Sprott D, Zheng C, Shih Y-P, et al. Neoantigen T634 Cell Receptor Gene Therapy in Pancreatic Cancer. New England Journal of Medicine 635 386(22):2112-9. doi: 10.1056/nejmoa2119662.
9. Raj D, Yang M-H, Rodgers D, Hampton E N, Begum J, Mustafa A, et al. Switchable Car-T Cells Mediate Remission in Metastatic Pancreatic Ductal Adenocarcinoma. Gut (2019) 68(6):1052-64. doi: 10.1136/gutjnl-2018-316595.
10. Beatty G L, O'Hara M H, Lacey S F, Torigian D A, Nazimuddin F, Chen F, et al. Activity of Mesothelin-Specific Chimeric Antigen Receptor T Cells against Pancreatic Carcinoma Metastases in a Phase 1 Trial. Gastroenterology (2018) 155(1):29-32. doi: 10.1053/j.gastro.2018.03.029.
11. Raj D, Nikolaidi M, Garces I, Lorizio D, Castro N M, Caiafa S G, et al. Ceacam7 Is an Effective Target for Car T-Cell Therapy of Pancreatic Ductal Adenocarcinoma. Clin Cancer Res (2021) 27(5):1538-52. Epub 2021 Jan. 23. doi: 10.1158/1078-0432.Ccr-19-2163.
12. Blum J S, Wearsch P A, Cresswell P. Pathways of Antigen Processing. Annu Rev Immunol (2013) 31:443-73. Epub 2013 Jan. 10. doi: 10.1146/annurev-immunol-032712-095910.
13. Oancea G, O'Mara M L, Bennett W F, Tieleman D P, Abele R, Tampe R. Structural Arrangement of the Transmission Interface in the Antigen Abc Transport Complex Tap. Proc Natl Acad Sci USA (2009) 106(14):5551-6. Epub 2009 Mar. 20. doi: 10.1073/pnas.0811260106.
14. Hillaireau H, Couvreur P. Nanocarriers' Entry into the Cell: Relevance to Drug Delivery. Cellular and Molecular Life Sciences (2009) 66(17):2873-96. doi: 10.1007/s00018-009-0053 z.
15. Behzadi S, Serpooshan V, Tao W, Hamaly M A, Alkawareck M Y, Dreaden E C, et al. Cellular Uptake of Nanoparticles: Journey inside the Cell. Chem Soc Rev (2017) 46(14):4218-44. doi: 10.1039/c6cs00636a.
16. Li Y, Wang J, Wientjes M G, Au J L. Delivery of Nanomedicines to Extracellular and Intracellular Compartments of a Solid Tumor. Adv Drug Deliv Rev (2012) 64(1):29-39. Epub 2011 May 17. doi: 10.1016/j.addr.2011.04.006.
17. Dang F W, Chen L, Madura K. Catalytically Active Proteasomes Function Predominantly in the Cytosol. J Biol Chem (2016) 291(36):18765-77. Epub 2016 Jul. 16. doi: 10.1074/jbc.M115.712406.
18. Blees A, Januliene D, Hofmann T, Koller N, Schmidt C, Trowitzsch S, et al. Structure of the Human Mhc-I Peptide-Loading Complex. Nature (2017) 551(7681):525-8. Epub 2017 Nov. 7. doi: 10.1038/nature24627.
19. Liepe J, Marino F, Sidney J, Jeko A, Bunting D E, Sette A, et al. A Large Fraction of Hla Class I Ligands Are Proteasome-Generated Spliced Peptides. Science (2016) 354(6310):354 8. Epub 2016 Nov. 16. doi: 10.1126/science.aaf4384.
20. Corr M, Slanetz A E, Boyd L F, Jelonek M T, Khilko S, al-Ramadi B K, et al. T Cell Receptor-Mhc Class I Peptide Interactions: Affinity, Kinetics, and Specificity. Science (1994) 265(5174):946-9. Epub 1994 Aug. 12. doi: 10.1126/science.8052850.
21. Schumacher T N, Schreiber R D. Neoantigens in Cancer Immunotherapy. Science (2015) 348(6230):69-74. Epub 2015 Apr. 4. doi: 10.1126/science.aaa4971.
22. Coulie P G, Van den Eynde B J, van der Bruggen P, Boon T. Tumour Antigens Recognized by T Lymphocytes: At the Core of Cancer Immunotherapy. Nat Rev Cancer (2014) 14(2):135-46. Epub 2014 Jan. 25. doi: 10.1038/nrc3670.
23. Spel L, Boelens J J, Nierkens S, Boes M. Antitumor Immune Responses Mediated by Dendritic Cells: How Signals Derived from Dying Cancer Cells Drive Antigen Cross Presentation. Oncoimmunology (2013) 2(11): e26403. Epub 2014 Feb. 1. doi: 10.4161/onci.26403.
24. Gulley J L, Madan R A, Pachynski R, Mulders P, Sheikh N A, Trager J, et al. Role of Antigen Spread and Distinctive Characteristics of Immunotherapy in Cancer Treatment. J Natl Cancer Inst (2017) 109(4). Epub 2017 Apr. 5. doi: 10.1093/jnci/djw261.
25. Raman V, Van Dessel N, Hall C L, Wetherby V E, Whitney S A, Kolewe E L, et al. Intracellular Delivery of Protein Drugs with an Autonomously Lysing Bacterial System Reduces Tumor Growth and Metastases. Nat Commun (2021) 12(1). doi: 10.1038/s41467 021-26367-9.
26. Cossart P, Sansonetti P J. Bacterial Invasion: The Paradigms of Enteroinvasive Pathogens. Science (2004) 304(5668):242-8. doi: 10.1126/science.1090124.
27. Pizarro-Cerdá J, Cossart P. Bacterial Adhesion and Entry into Host Cells. Cell (2006) 124(4):715-27. Epub 2006 Feb. 25. doi: 10.1016/j.cell.2006.02.012.
28. Forbes N S, Munn L L, Fukumura D, Jain R K. Sparse Initial Entrapment of Systemically Injected Salmonella Typhimurium Leads to Heterogeneous Accumulation within Tumors. Cancer Res (2003) 63(17):5188-93. Epub 2003 Sep. 23.
29. Leschner S, Westphal K, Dietrich N, Viegas N, Jablonska J, Lyszkiewicz M, et al. Tumor Invasion of Salmonella Enterica Serovar Typhimurium Is Accompanied by Strong Hemorrhage Promoted by Tnf-Alpha. PLOS One (2009) 4(8): e6692. Epub 2009 Aug. 21. doi: 10.1371/journal.pone.0006692.
30. Hyun J, Jun S, Lim H, Cho H, You S-H, Ha S-J, et al. Engineered Attenuated <I>Salmonella Typhimurium</I> Expressing Neoantigen Has Anticancer Effects. ACS Synthetic biology (2021) 10(10):2478-87. doi: 10.1021/acssynbio.1c00097.
31. Gurbatri C R, Lia I, Vincent R, Coker C, Castro S, Treuting P M, et al. Engineered Probiotics for Local Tumor Delivery of Checkpoint Blockade Nanobodies. Sci Transl Med (2020) 12(530): eaax0876. doi: 10.1126/scitranslmed.aax0876.
32. Murakami T, Hiroshima Y, Zhang Y, Zhao M, Kiyuna T, Hwang H K, et al. Tumor Targeting Salmonella Typhimurium A1-R Promotes Tumoricidal Cd8(+) T Cell Tumor Infiltration and Arrests Growth and Metastasis in a Syngeneic Pancreatic-Cancer Orthotopic Mouse Model. J Cell Biochem (2018) 119(1):634-9. Epub 2017 Jun. 20. doi: 10.1002/jcb.26224.
33. Raman V, Van Dessel N, Hall C L, Wetherby V E, Whitney S A, Kolewe E L, et al. Intracellular Delivery of Protein Drugs with an Autonomously Lysing Bacterial System Reduces Tumor Growth and Metastases. Nat Commun (2021) 12(1):6116. Epub 2021 Oct. 23. doi: 10.1038/s41467-021-26367-9.
34. Raman V, Van Dessel N, O'Connor O M, Forbes N S. The Motility Regulator Flhdc Drives Intracellular Accumulation and Tumor Colonization of Salmonella. Journal for immunotherapy of cancer (2019) 7(1):44. Epub 2019 Feb. 14. doi: 10.1186/s40425-018-0490z.
35. Low K B, Ittensohn M, Le T, Platt J, Sodi S, Amoss M, et al. Lipid a Mutant Salmonella With Suppressed Virulence and Tnfalpha Induction Retain Tumor-Targeting in Vivo. Nature biotechnology (1999) 17:37-41. doi: 10.1038/5205.
36. Clairmont C, Lee K C, Pike J, Ittensohn M, Low K B, Pawelek J, et al. Biodistribution and Genetic Stability of the Novel Antitumor Agent Vnp20009, a Genetically Modified Strain of Salmonella Typhimurium. J Infect Dis (2000) 181(6):1996-2002. Epub 2000 Jun. 6. doi: 10.1086/315497.
37. Carswell E A, Old L J, Kassel R L, Green S, Fiore N, Williamson B. Endotoxin-Induced Serum Factor That Causes Necrosis of Tumors. Proc Natl Acad Sci USA (1975) 72(9):3666
70. doi: 10.1073/pnas.72.9.3666.
38. Luo X, Li Z J, Lin S, Le T, Ittensohn M, Bermudes D, et al. Antitumor Effect of Vnp20009, an Attenuated Salmonella, in Murine Tumor Models. Oncol Res (2001) 12(11 12):501-8.
39. Thamm D H, Kurzman I D, King I, Li Z, Sznol M, Dubielzig R R, et al. Systemic Administration of an Attenuated, Tumor-Targeting Salmonella Typhimurium to Dogs with Spontaneous Neoplasia: Phase I Evaluation. Clinical cancer research: an official journal of the American Association for Cancer Research (2005) 11:4827-34. doi: 10.1158/1078 0432.CCR-04-729 2510.
40. Toso J F, Gill V J, Hwu P, Marincola F M, Restifo N P, Schwartzentruber D J, et al. Phase I Study of the Intravenous Administration of Attenuated Salmonella Typhimurium to Patients With Metastatic Melanoma. Journal of clinical oncology: official journal of the American Society of Clinical Oncology (2002) 20:142-52.
41. Leschner S, Westphal K, Dietrich N, Viegas N, Jablonska J, Lyszkiewicz M, et al. Tumor Invasion of Salmonella Enterica Serovar Typhimurium Is Accompanied by Strong Hemorrhage Promoted by Tnf-Alpha. PloS one (2009) 4: e6692. doi: 10.1371/journal.pone.0006692.
42. Kasinskas R W, Forbes N S. Salmonella Typhimurium Specifically Chemotax and Proliferate in Heterogeneous Tumor Tissue in Vitro. Biotechnology and Bioengineering (2006) 94(4):710-21. doi: 10.1002/bit.20883.
43. Kasinskas R W, Forbes N S. Salmonella Typhimurium Lacking Ribose Chemoreceptors Localize in Tumor Quiescence and Induce Apoptosis. Cancer research (2007) 67:3201-9. doi; 10.1158/0008-5472.CAN-06-2618.
44. Sznol M, Lin S L, Bermudes D, Zheng L M, King I. Use of Preferentially Replicating Bacteria for the Treatment of Cancer. J Clin Invest (2000) 105(8):1027-30.
45. Pham T N D, Shields M A, Spaulding C, Principe D R, Li B, Underwood P W, et al. Preclinical Models of Pancreatic Ductal Adenocarcinoma and Their Utility in Immunotherapy Studies. Cancers (2021) 13(3):440. doi: 10.3390/cancers13030440.
46. Feig C, Jones J O, Kraman M, Wells R J B, Deonarine A, Chan D S, et al. Targeting Cxel12 from Fap-Expressing Carcinoma-Associated Fibroblasts Synergizes with Anti-Pd-L1 Immunotherapy in Pancreatic Cancer. Proceedings of the National Academy of Sciences (2013) 110(50):20212-7. doi: 10.1073/pnas.1320318110.
47. Deng W, Lira V, Hudson T E, Lemmens B E, Hanson W G, Flores R, et al. Recombinant Listeria Promotes Tumor Rejection by Cd8+ T Cell-Dependent Remodeling of the Tumor Microenvironment. Proceedings of the National Academy of Sciences (2018) 115(32):8179 84. doi: 10.1073/pnas. 1801910115.
48. Johnson S A, Ormsby M J, Wessel H M, Hulme H E, Bravo-Blas A, McIntosh A, et al. Monocytes Mediate <I>Salmonella Typhimurium</I>-Induced Tumor Growth Inhibition in a Mouse Melanoma Model. European Journal of Immunology (2021) 51(12):3228-38. doi: 10.1002/eji.202048913.
49. Li M, Lu M, Lai Y, Zhang X, Li Y, Mao P, et al. Inhibition of Acute Leukemia with Attenuated Salmonella Typhimurium Strain Vnp20009. Biomedicine & pharmacotherapy Biomedecine & pharmacotherapie (2020) 129:110425. Epub 2020 Jun. 23. doi: 10.1016/j.biopha.2020.110425.
50. Mei Y, Zhao L, Liu Y, Gong H, Song Y, Lei L, et al. Combining DNA Vaccine and Aida-1 in Attenuated <I>Salmonella</I> Activates Tumor-Specific Cd4<Sup>+</Sup> and Cd8<Sup>+</Sup> T-Cell Responses. Cancer immunology research (2017) 5(6):503-14. doi: 10.1158/2326-6066.cir-16-0240-t.
51. Lee C-H, Hsieh J-L, Wu C-L, Hsu P-Y, Shiau A-L. T Cell Augments the Antitumor Activity of Tumor-Targeting Salmonella. Applied microbiology and biotechnology (2011) 90(4):1381-8. doi: 10.1007/s00253-011-3180-z.
52. Bos R, Sherman L A. Cd4+ T-Cell Help in the Tumor Milieu Is Required for Recruitment and Cytolytic Function of Cd8+ T Lymphocytes. Cancer Research (2010) 70(21):8368-77. doi: 10.1158/0008-5472.can-10-1322.
53. Novy P, Quigley M, Huang X, Yang Y. Cd4 T Cells Are Required for Cd8 T Cell Survival During Both Primary and Memory Recall Responses. J Immunol (2007) 179(12):8243-51. Epub 2007 Dec. 7. doi: 10.4049/jimmunol.179.12.8243.
54. Laidlaw B J, Craft J E, Kaech S M. The Multifaceted Role of Cd4+ T Cells in Cd8+ T Cell Memory. Nat Rev Immunol (2016) 16(2):102-11. doi: 10.1038/nri.2015.10.
55. Emmerich J, Mumm J B, Chan I H, LaFace D, Truong H, McClanahan T, et al. Il-10 Directly Activates and Expands Tumor-Resident Cd8(+) T Cells without De Novo Infiltration from Secondary Lymphoid Organs. Cancer Res (2012) 72(14):3570-81. Epub 2012 May 15. doi: 10.1158/0008-5472.CAN-12-0721.
56. Curran M A, Kim M, Montalvo W, Al-Shamkhani A, Allison J P. Combination Ctla-4 Blockade and 4-1bb Activation Enhances Tumor Rejection by Increasing T-Cell Infiltration, Proliferation, and Cytokine Production. PLOS One (2011) 6(4): e19499. Epub 2011 May 12. doi: 10.1371/journal.pone.0019499.
57. Chang L Y, Lin Y C, Mahalingam J, Huang C T, Chen T W, Kang C W, et al. Tumor-Derived Chemokine Cc15 Enhances Tgf-Beta-Mediated Killing of Cd8(+) T Cells in Colon Cancer by T-Regulatory Cells. Cancer Res (2012) 72(5):1092-102. Epub 2012 Jan. 28. doi: 10.1158/0008-5472.CAN-11-2493.
58. Matsuo M, Nagata Y, Sato E, Atanackovic D, Valmori D, Chen Y T, et al. Ifn-Gamma Enables Cross-Presentation of Exogenous Protein Antigen in Human Langerhans Cells by Potentiating Maturation. Proc Natl Acad Sci USA (2004) 101(40):14467-72. Epub 2004 Sep. 24. doi: 10.1073/pnas.0405947101.
59. Principe N, Kidman J, Goh S, Tilsed C M, Fisher S A, Fear V S, et al. Tumor Infiltrating Effector Memory Antigen-Specific Cd8(+) T Cells Predict Response to Immune Checkpoint Therapy. Front Immunol (2020) 11:584423. Epub 2020 Dec. 3. doi: 10.3389/fimmu.2020.584423.
60. Whitmire J K, Eam B. Whitton J L. Tentative T Cells: Memory Cells Are Quick to Respond, but Slow to Divide. PLOS Pathogens (2008) 4(4): e1000041. doi: 10.1371/journal.ppat. 1000041.
61. CDC. Immunization [website]. (2017) [updated Mar. 17, 2017; cited 2020 2020]. Available from: cdc.gov/nchs/fastats/immunize.htm.
62. Bossi G, Griffiths G M. Ctl Secretory Lysosomes: Biogenesis and Secretion of a Harmful Organelle. Semin Immunol (2005) 17(1):87-94. Epub 2004 Dec. 8. doi: 10.1016/j.smim.2004.09.007.
63. de Saint Basile G, Menasche G, Fischer A. Molecular Mechanisms of Biogenesis and Exocytosis of Cytotoxic Granules. Nat Rev Immunol (2010) 10(8):568-79. Epub 2010 Jul. 17. doi: 10.1038/nri2803.
64. Voskoboinik I, Smyth M J, Trapani J A. Perforin-Mediated Target-Cell Death and Immune Homeostasis. Nat Rev Immunol (2006) 6(12):940-52. Epub 2006 Nov. 25. doi: 10.1038/nri1983.
65. Martinez-Lostao L, Anel A, Pardo J. How Do Cytotoxic Lymphocytes Kill Cancer Cells? Clin Cancer Res (2015) 21(22):5047-56. Epub 2015 Nov. 15. doi: 10.1158/1078-0432.CCR-15-0685.
66. Wang R, Natarajan K, Margulies D H. Structural Basis of the Cd8 Alpha Beta/Mhc Class I Interaction: Focused Recognition Orients Cd8 Beta to a T Cell Proximal Position. J Immunol (2009) 183(4):2554-64. Epub 2009 Jul. 25. doi: 10.4049/jimmunol.0901276.
67. Schnurr M, Scholz C, Rothenfusser S, Galambos P, Dauer M, Robe J, et al. Apoptotic Pancreatic Tumor Cells Are Superior to Cell Lysates in Promoting Cross-Priming of Cytotoxic T Cells and Activate Nk and Gammadelta T Cells. Cancer Res (2002) 62(8):2347-52. Epub 2002 Apr. 17.
68. Strome S E, Voss S, Wilcox R, Wakefield T L, Tamada K, Flies D, et al. Strategies for Antigen Loading of Dendritic Cells to Enhance the Antitumor Immune Response. Cancer Res (2002) 62(6):1884-9. Epub 2002 Mar. 26.
69. Blachere N E, Darnell R B, Albert M L. Apoptotic Cells Deliver Processed Antigen to Dendritic Cells for Cross-Presentation. PLOS Biol (2005) 3(6): e185. Epub 2005 Apr. 21. doi: 10.1371/journal.pbio.0030185.
70. Bachmann M F, Wolint P, Schwarz K, Jager P, Oxenius A. Functional Properties and Lineage Relationship of Cd8+ T Cell Subsets Identified by Expression of Il-7 Receptor Alpha and Cd621. J Immunol (2005) 175(7):4686-96. Epub 2005 Sep. 24.
71. Mahnke Y D, Brodie T M, Sallusto F, Roederer M, Lugli E. The Who's Who of T-Cell Differentiation: Human Memory T-Cell Subsets. Eur J Immunol (2013) 43(11):2797-809. Epub 2013 Nov. 22. doi: 10.1002/eji.201343751.
72. Pedicord V A, Montalvo W, Leiner I M, Allison J P. Single Dose of Anti-Ctla-4 Enhances Cd8+ T-Cell Memory Formation, Function, and Maintenance. Proc Natl Acad Sci USA (2011) 108(1):266-71. Epub 2010 Dec. 22. doi: 10.1073/pnas. 1016791108.
73. Wei S C, Levine J H, Cogdill A P, Zhao Y, Anang N A S, Andrews M C, et al. Distinct Cellular Mechanisms Underlie Anti-Ctla-4 and Anti-Pd-1 Checkpoint Blockade. Cell (2017) 170(6):1120-33 e17. Epub 2017 Aug. 15. doi: 10.1016/j.cell.2017.07.024.

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. A non-pathogenic bacterial cell expressing an exogenous immunogenic protein intracellularly, wherein the cell comprises a lysis gene or lysis cassette operably linked to an intracellularly induced Salmonella promoter.

2. The non-pathogenic bacterial cell of claim 1, wherein the expressed protein is coded for by an expression plasmid.

3. The non-pathogenic bacterial cell of claim 1, wherein the protein is a viral, bacterial, fungal or protozoic protein.

4. The cell of claim 1, wherein the immunogenic protein is found in one or more of the following vaccines that to immunize against anthrax (AVA (BioThrax); cholera (Vaxchora), COVID-19 (Pfizer-BioNTech; Moderna; Johnson & Johnson's Janssen), diptheria (DTaP (Daptacel, Infanrix); Td (Tenivac, generic); DT (-generic-); Tdap (Adacel, Boostrix); DTaP-IPV (Kinrix, Quadracel); DTaP-HepB-IPV (Pediarix); DTaP-IPV/Hib (Pentacel)), hepatitis A (HepA (Havrix, Vaqta); HepA-HepB (Twinrix)), Hepatitis B (HepB (Engerix-B, Recombivax HB, Heplisav-B); DTaP-HepB-IPV (Pediarix); HepA-HepB (Twinrix)), Haemophilus influenzae type b (Hib) (Hib (ActHIB, PedvaxHIB, Hiberix); DTaP-IPV/Hib (Pentacel)), Human Papillomavirus (HPV) (HPV9 (Gardasil 9) (For scientific papers, the preferred abbreviation is 9vHPV)), Seasonal Influenza (Flu) (IIV* (Afluria, Fluad, Flublok, Flucelvax, FluLaval, Fluarix, Fluvirin, Fluzone, Fluzone High-Dose, Fluzone Intradermal; there are various acronyms for inactivated flu vaccines-IIV3, IIV4, RIV3, RIV4 and cclIV4; LAIV (FluMist)), Japanese Encephalitis (JE (Ixiaro)), Measles (MMR (M-M-R II); MMRV (ProQuad)), Meningococcal (MenACWY (Menactra, Menveo); MenB (Bexsero, Trumenba)), Mumps (MMR (M-M-R II); MMRV (ProQuad)), Pertussis (DTaP (Daptacel, Infanrix); Tdap (Adacel, Boostrix); DTaP-IPV (Kinrix, Quadracel); DTaP-HepB-IPV (Pediarix); DTaP-IPV/Hib (Pentacel)), Pneumococcal (PCV13 (Prevnar13); PPSV23 (Pneumovax 23)), Polio (Polio (Ipol); DTaP-IPV (Kinrix, Quadracel); DTaP-HepB-IPV (Pediarix); DTaP-IPV/Hib (Pentacel)), Rabies (Rabies (Imovax Rabies, RabAvert)), Rotavirus (RV1 (Rotarix); RV5 (RotaTeq)), Rubella (MMR (M-M-R II); MMRV (ProQuad)), Shingles (RZV (Shingrix)), Smallpox (Vaccinia (ACAM2000)), Tetanus (DTaP (Daptacel, Infanrix); Td (Tenivac, generic), DT (-generic-), Tdap (Adacel, Boostrix), DTaP-IPV (Kinrix, Quadracel), DTaP-HepB-IPV (Pediarix), DTaP-IPV/Hib (Pentacel)), Typhoid Fever (Typhoid Oral (Vivotif); Typhoid Polysaccharide (Typhim Vi)), Varicella (VAR (Varivax); MMRV (ProQuad)), Covid-19 (Novavax or ImmunityBio) and/or Yellow Fever (YF (YF-Vax)).

5. The cell of claim 1, wherein the cell comprises inducible expression of flagella.

6. The cell of claim 1, wherein expression of SseJ has been reduced.

7-8. (canceled)

9. The cell of claim 1, wherein the bacterial cell is an intratumoral bacteria cell.

10. The cell of claim 1, wherein the bacterial cell is a Clostridium, Bifidus, Escherichia coli or Salmonella cell.

11. The cell of claim 5, wherein the lysis cassette is Lysin E from phage phiX174, the lysis cassette of phage iEPS5, or the lysis cassette from lambda phage.

12. The cell of claim 5, wherein the intracellularly induced Salmonella promoter is for one of the genes in Salmonella pathogenicity island 2 type III secretion system (SPI2-T3SS) selected from the group SpiC/SsaB, SseF, SseG, SseI, SseJ, SseK1, SseK2, SifA, SifB, PipB, PipB2, SopD2, GogB, SseL, SteC, SspH1, SspH2, or SirP.

13. The cell of claim 1, wherein the cell does not comprise endogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL expression.

14. The cell of claim 1, wherein the cell comprises an exogenous inducible promoter operably linked to an endogenous or exogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL gene.

15-17. (canceled)

18. A composition comprising a population of cells of claim 1 and a pharmaceutically acceptable carrier.

19. A method to selectively colonize a tumor and/or tumor associated cells comprising administering a population of the bacterial cells of claim 1 to a subject in need thereof, wherein the exogenous immunogenic protein is not from the same species as the subject.

20. The method of claim 19, wherein the tumor associated cells are intratumoral immune cells or stromal cells within tumors.

21. A method to treat cancer comprising administering to subject in need thereof an effective amount of a population of the bacterial cells of claim 1 so as to treat said cancer, wherein the subject has previously been exposed to the exogenous immunogenic protein, wherein the exogenous immunogenic protein is not from the same species as the subject.

22-27. (canceled)

28. A method to provide an anti-tumor, vaccine associated, CD8 T or CD4 cell specific immune response comprising administering an effective amount of a population of the bacterial cells of claim Ito a subject in need thereof, wherein the subject has previously been exposed to the exogenous immunogenic protein, wherein the exogenous immunogenic protein is not from the same species as the subject.

29. The method of claim 28, wherein the anti-tumor, CD8 T cell specific immune response is an anti-tumor, memory CD8T or CD4 T cell specific immune response.

30-31. (canceled)

32. The method of claim 19, wherein the bacterial cells deliver said vaccine derived peptide to said tumor, tumor associated cells, cancer, or metastases.

33. The method of claim 19, wherein the tumor, tumor associated cells, cancer, or metastases are a lung, liver, kidney, breast, prostate, pancreatic, colon, head and neck, ovarian and/or gastroenterological tumor, tumor associated cells, cancer or metastases.
